SEQUENCE LISTING (5 sequences)

SEQ ID NO: 1 - DNA, 20 bp, Artificial
  synthetic
cgctgaacttggcgtggtcc

SEQ ID NO: 2 - DNA, 25 bp, Artificial
  synthetic
ggctgccctgctaagaaaattctcc

SEQ ID NO: 3 - protein, 5 aa, Artificial
  synthetic
GAPDH

SEQ ID NO: 4 - DNA, 21 bp, Artificial
  synthetic
acacccactcctccacctttg

SEQ ID NO: 5 - DNA, 21 bp, Artificial
  synthetic
caccaccctgttgctgtagcc

(19) United States
(12) Patent Application Publication
(10) Pub. No.: US 2021/0244721 A1
(43) Pub. Date: Aug. 12, 2021

(54) SMALL MOLECULE ENTEROVIRUS INHIBITORS AND USES THEREOF

(71) Applicant: Arizona Board of Regents on Behalf of the University of Arizona, Tucson, AZ (US)

(72) Inventors: Jun Wang, Tucson, AZ (US); Rami Ghassan Musharrafieh, Tucson, AZ (US); Chunlong Ma, Tucson, AZ (US); Jiantao Zhang, Tucson, AZ (US); Yanmei Hu, Tucson, AZ (US)

(21) Appl. No.: 17/162,670

(22) Filed: Jan. 29, 2021

Related U.S. Application Data
(60) Provisional application No. 62/968,803, filed on Jan. 31, 2020.

(51) Int. Cl. A61K 31/47; A61P 31/14; A61K 31/4709; A61K 31/4748; A61K 45/06; A61K 31/5377; A61K 31/506; A61K 31/496

(52) U.S. Cl. CPC A61K 31/47; A61P 31/14; A61K 31/4709; A61K 31/496; A61K 45/06; A61K 31/5377; A61K 31/506; A61K 31/4748

(57) ABSTRACT

This invention is in the field of medicinal chemistry. In particular, the invention relates to a new class of small-molecules having a quinoline (or similar) structure which function as antagonists of androgen receptor activity, and their use as therapeutics for the treatment of cancer (e.g., castration-resistant prostate cancer) and other conditions characterized with androgen receptor activity and/or androgen receptor expression.

### CROSS REFERENCE TO RELATED APPLICATION

This application claims priority to and the benefit of U.S. Provisional Application No. 62/968,803, filed Jan. 31, 2020, which is hereby incorporated by reference in its entirety.

### STATEMENT REGARDING FEDERALLY SPONSORED RESEARCH OR DEVELOPMENT

This invention was made with government support under Grant Nos. R33 AI119187 and R21 AI144887 awarded by National Institutes of Health. The government has certain rights in the invention.

### FIELD OF THE INVENTION

This invention is in the field of medicinal chemistry. In particular, the invention relates to a new class of small-molecule compounds having a quinoline (or similar) structure which function as enterovirus inhibitors, and their use as therapeutics for the treatment of conditions characterized with enterovirus activity (e.g., respiratory infections, neurological conditions).

### INTRODUCTION

Enterovirus, a genus from the Picornaviridae family, is implicated in more than 10 million illnesses in the United States each year (see, J. Baggen, et al., Nat. Rev. Microbiol. 16 (2018) 368-381). Among the various species of enteroviruses, 7 commonly infect humans: enterovirus A-D and rhinoviruses A-C. Since the successful eradication of poliovirus (PV) in the developed world, the main cause of enteroviral diseases are associated with non-polio enterovirus (EV) infections. Classification of enterovirus species are based principally on genomic and viral protein similarities. However, many species can have distinct cell surface receptors for entry, tissue tropism and pathogenesis. Enterovirus A serotype EV-A71 have been associated with large-scale epidemics since the early 1970s in Europe, the United States, Australia, and most aggressively, in the Asia-Pacific region (see, T. Solomon, et al., Lancet Infect. Dis. 10 (2010) 778-790). EV-A71 is associated with many neurological complications similar to other EVs, including acute flaccid paralysis (AFM), aseptic meningitis, encephalitis or cardiorespiratory illness, but in most cases, EV-A71 is known for causing Hand Foot and Mouth Disease (HFMD) in children (see, M. H. Ooi, et al., Lancet Neurol. 9 (2010) 1097-1105). Enterovirus B serotype coxsackievirus B3 (CVB3) is commonly associated with viral heart disease, and may play a role in the onset of juvenile diabetes mellitus (see, C. Baboonian, et al., Curr. Opin. Microbiol. Immunol. 223 (1997) 31-52).

As of date, the most mysterious EV is the serotype enterovirus D68 (EV-D68) from the enterovirus D species. Unlike most enteroviruses, EV-D68 was largely unstudied until an unexpected epidemic occurred in 2014 in the United States (see, C. C. Holm-Hansen, et al., Lancet Infect. Dis. 16 (2016) E64-E75). Since then the outbreak appears to be reoccurring in a biennial pattern. Initial clinical manifestation of EV-D68 infection resembles rhinovirus infection, with mild-to-severe upper respiratory infection in children between the ages of 1-11 (see, C. M. Midgley, et al., Lancet Respir. Med. 3 (2015) 879-887). Unlike rhinovirus infection, recent EV-D68 strains have been linked with a large number of patients developing acute flaccid myelitis, or AFM (see, A. L. Greninger, et al., Lancet Infect. Dis. 15 (2015) 671-682).

All EVs are non-enveloped positive-sense RNA viruses that are encapsulated by viral proteins VP1-4. The viral genome can be translated directly into a polyprotein which is processed by viral 2A and 3C proteases. Genome replication is mediated by the RNA-dependent RNA polymerase (RDRP) 3D^{pol} in replication organelles. The multifunctional viral protein 2C is involved in membrane rearrangement (see, N. L. Teterina, et al., J. Virol. 71 (1997) 8962-8972), uncoating (see, E. Asare, et al., J. Virol. 90 (2016) 6174-6186), assembly see, N. L. Teterina, et al., J. Virol. 71 (1997) 8962-8972), uncoating (see, E. Asare, et al., J. Virol. 90 (2016) 6174-6186; C. L. Wang, et al., J. Virol. 86 (2012) 9964-9975), as well as viral RNA replication (see, D. J. Barton, et al., J. Virol. 71 (1997) 8482-8489). 2C contains Walker A, B, and C motifs that form a conserved ATPase domain that has helicase activity in vitro (see, H. J. Xia, et al., Plos Pathog. 11 (2015). 2C also contains an N-terminal membrane-binding domain as well as a C-terminal domain that may play a role in the formation of dimers or higher oligomeric species (see, H. X. Guan, et al., Plos Pathog. 14 (2018)). Given the multifunctional roles of 2C in viral replication, it appears to be a high profile antiviral drug target (see, R. Musharrafieh, et al., J. Med. Chem. 62 (2019) 4074-4090).

Until vaccines can be developed, antivirals are required to alleviate the burden of disease and reduce fatalities associated with non-polio enterovirus infections. As of date, no antiviral is approved for the treatment of any enterovirus infection. The most promising candidates are direct-acting antivirals targeting viral proteins such as viral VP1 capsid protein, 2A and 3C proteases, 2C protein, and the viral 3D polymerase (see, J. Baggen, et al, Nat. Rev. Microbiol. 16 (2018) 368-381; J. Y. Lin, et al., J. Biomed. Sci. 26 (2019) 65; R. Musharrafieh, et al., J. Virol. 93 (2019), e02221-18; A. Egorova, et al., E. J. Med. Chem. 178 (2019) 606-622; C. Ma, et al., ACS Infect. Dis. 5 (2019) 1952-1962). The viral 2C protein is a conserved viral protein, and has been validated as a drug target by a number of potent antivirals (see, R. Musharrafieh, et al., J. Med. Chem. 62 (2019) 4074-4090; L. Bauer, et al., Curr. Opin. Virol. 24 (2017) 1-8). Dibucaine, a quinoline derivative commonly used as a topical anesthetic, was found to inhibit the replication of CVB3 by targeting the viral 2C protein (see, R. Ulferts, et al., Antimicrob. Agents. Chemother. 60 (2016) 2627-2638). However, it was found that dibucaine was only moderately effective against other enteroviruses such as EV-A71 and EV-D68 (see, R. Ulferts, et al., Antimicrob. Agents. Chemother. 60 (2016) 2627-2638).

As such, improved agents for treating and/or preventing enterovirus based conditions are needed.

The present invention addresses this need.

### SUMMARY OF THE INVENTION

Enterovirus D68 (EV-D68) is an atypical non-polio enterovirus that mainly infected the respiratory system of humans, leading to moderate to severe respiratory diseases. In rare cases, EV-D68 can spread to the central nervous system and causes paralysis in infected patients, especially young children and immunocompromised individuals. There is currently no approved vaccine or antiviral available for the prevention and treatment of EV-D68. Experiments conducted during the course of developing embodiments for the present invention aimed to improve the antiviral potency and selectivity of a previously reported EV-D68 inhibitor, dibucaine, through structure-activity relationship studies. In total, an initial sixty compounds were synthesized and tested against EV-D68 using the viral cytopathic effect (CPE) assay. Three compounds 10a, 12a, and 12c were identified to have significantly improved potency (EC₅₀<1 μM) and a high selectivity index (SI>180) compared to dibucaine against five different strains of EV-D68 viruses. These compounds also showed potent antiviral activity in neuronal cells such as A172 and SH-SYSY cells, indicating use for the treatment of both respiratory infection as well as neuronal infection.

Non-polio enteroviruses such as enterovirus A71 (EV-A71), EV-D68, and coxsackievirus B3 (CVB3) are significant human pathogens with disease manifestations ranging from mild flu-like symptoms to more severe encephalitis, myocarditis, acute flaccid paralysis/myelitis, and even death. There are currently no effective antivirals to prevent or treat non-polio enterovirus infection. Additional experiments resulted in development of potent and broad-spectrum antivirals against these non-polio enteroviruses. Starting from the initial lead compounds that had potent antiviral activity against EV-D68, 43 analogs were synthesized and profiled their broad-spectrum antiviral activity against additional EV-D68, EV-A71, and CVB3 viruses. Promising candidates were also selected for mouse microsomal stability test to prioritize lead compounds for future in vivo mouse model studies. Collectively, this multi-parameter optimization process revealed a promising lead compound 6aw that showed single-digit to submicromolar EC₅₀ values against two EV-D68 strains (US/KY and US/MO), two EV-A71 strains (Tainan and US/AK), and one CVB3 strain, with a high selectivity index. Encouragingly, 6aw was stable in mouse microsomes with a half-life of 114.7 minutes. Overall, 6aw represents one of the most potent broad-spectrum antiviral against non-polio enteroviruses, rendering it a promising lead candidate for non-polio enteroviruses with translational potential.

Accordingly, the present invention relates to a new class of small-molecule compounds having a quinoline (or similar) structure which function as enterovirus inhibitors, and their use as therapeutics for the treatment of conditions characterized with enterovirus activity (e.g., respiratory infections, neurological conditions).

Accordingly, the present invention contemplates that exposure of animals (e.g., humans) suffering from a condition characterized with enterovirus activity to therapeutically effective amounts of drug(s) having a quinoline (or similar) structure will inhibit the enterovirus growth and/or activity outright and/or render such cells as a population more susceptible to other antiviral therapeutic agents. The present invention contemplates that such enterovirus antagonists satisfy an unmet need for the treatment of multiple types of infection associated with enterovirus activity, either when administered as monotherapy or when administered in a temporal relationship with additional agent(s) (e.g., antiviral agents).

The Applicants have found that certain quinoline (or similar) compounds function as antagonists of enterovirus activity and/or expression, and serve as therapeutics for the treatment of conditions associated with enterovirus infection and other diseases.

Certain quinoline (or similar) compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, quinoline (or similar) compounds encompassed within Formulas I is provided:

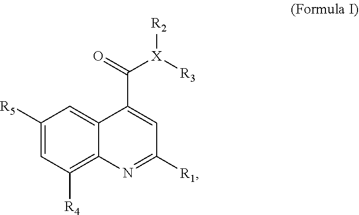

including pharmaceutically acceptable salts, solvates, and/or prodrugs thereof.

In some embodiments, X is either O or N.

Formula I is not limited to a particular chemical moiety for X, R1, R2, R3, R4 and R5. In some embodiments, the particular chemical moiety for X, R1, R2, R3, R4 and R5 independently include any chemical moiety that permits the resulting compound to inhibit enterovirus (e.g., non-polio enterovirus) (e.g., EV-D68, EV-A71, CVB3) activity and/or expression.

In some embodiments, R1 is selected from hydrogen,

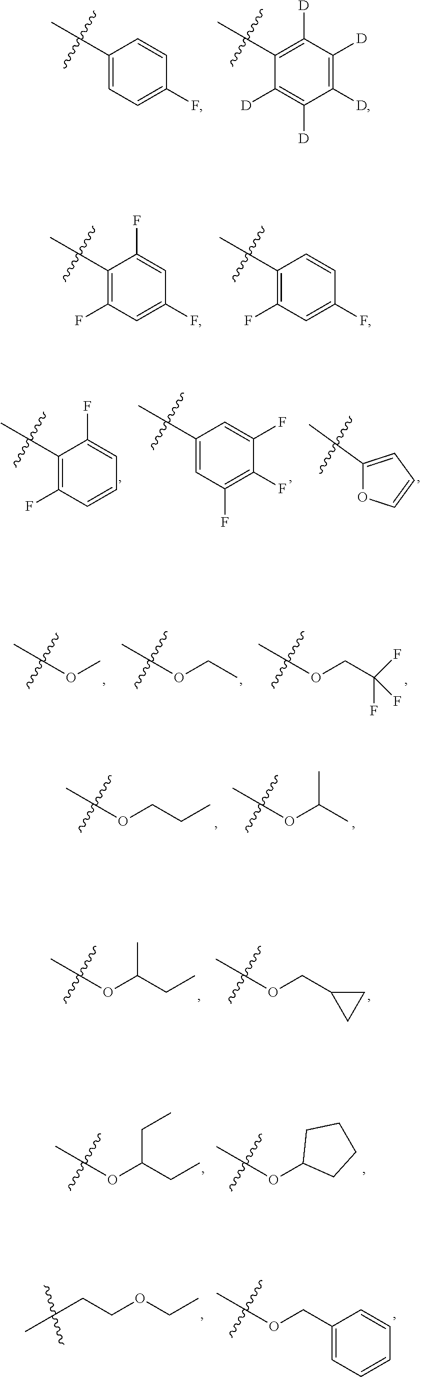

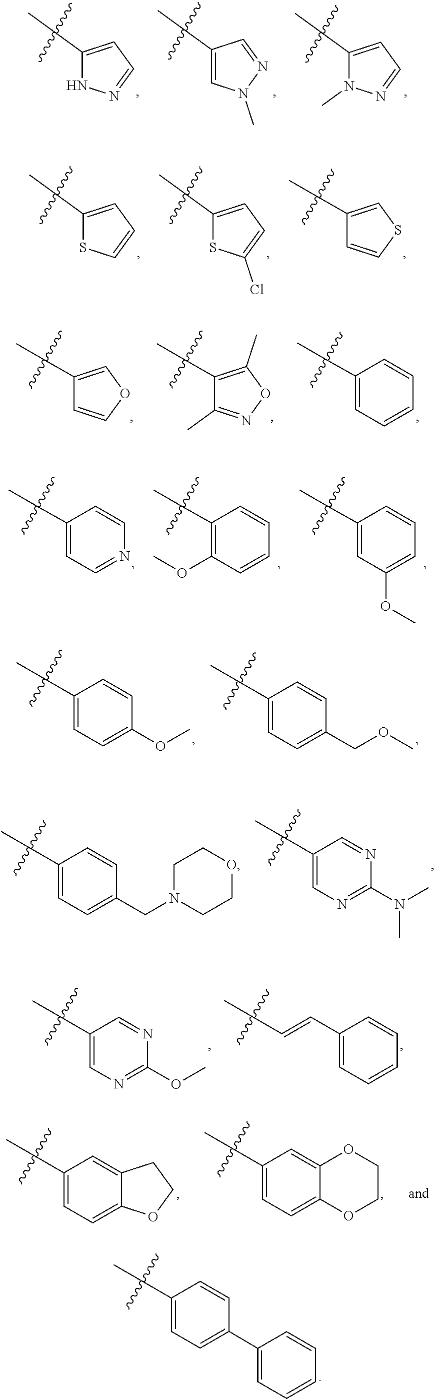

In some embodiments, R2 and R3 are independently selected from the group consisting of hydrogen,

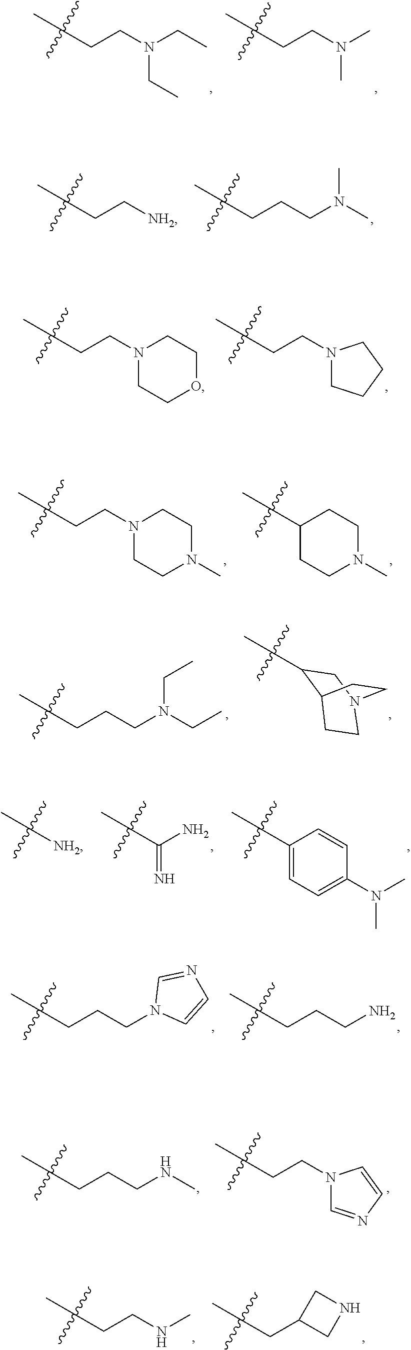

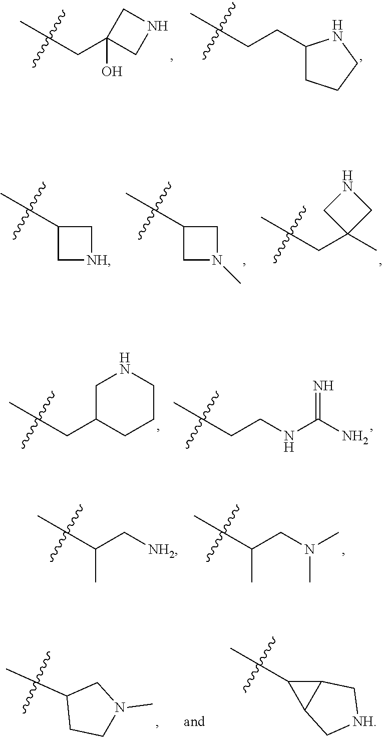

In some embodiments, R2 and R3 combine such that

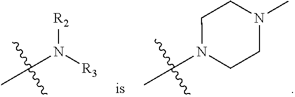

In some embodiments, R4 is selected from hydrogen and halogen (e.g., Chlorine).

In some embodiments, R5 is selected from hydrogen, halogen (e.g., fluorine, chlorine), and methoxy.

In some embodiments, compounds shown in Tables 2 (not including dibucaine), 3, 4, 5, 6 and 9 are contemplated for Formula I.

In a particular embodiment, quinoline (or similar) compounds encompassed within Formulas II is provided:

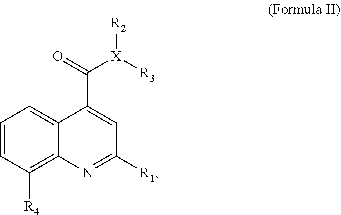

including pharmaceutically acceptable salts, solvates, and/or prodrugs thereof.

Formula II is not limited to a particular chemical moiety for X, R1, R2, R3, and R4. In some embodiments, the particular chemical moiety for X, R1, R2, R3, and R4 independently include any chemical moiety that permits the resulting compound to inhibit enterovirus (e.g., non-polio enterovirus) (e.g., EV-D68, EV-A71, CVB3) activity and/or expression.

In some embodiments, X is either O or N.

In some embodiments, R1 is selected from

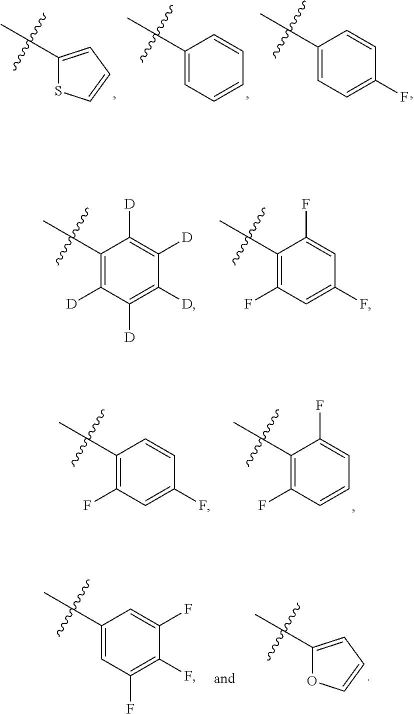

In some embodiments, R2 and R3 are independently selected from hydrogen,

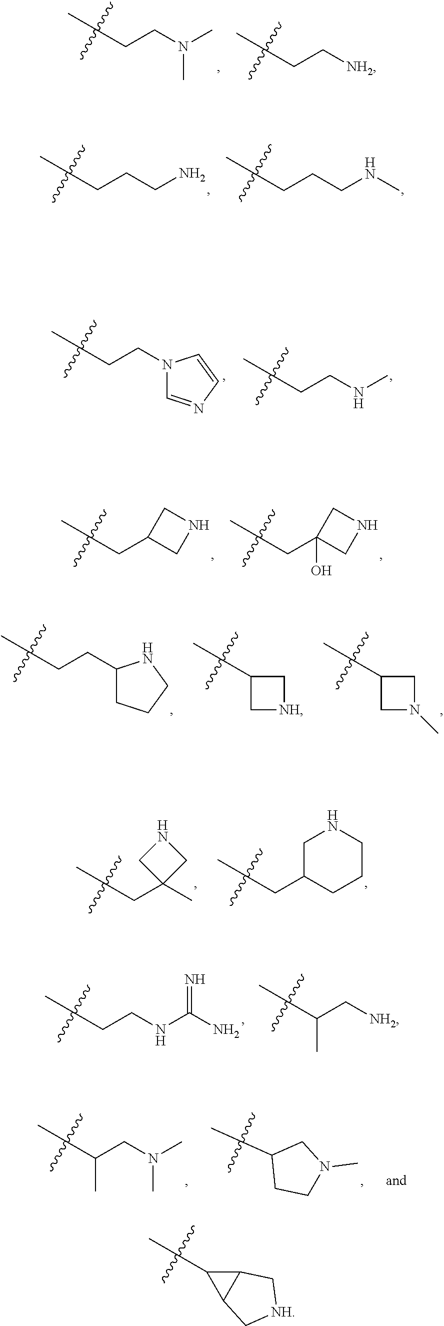

In some embodiments, R4 is selected from hydrogen and halogen (e.g., Chlorine).

In some embodiments, compounds shown in Table 9 are contemplated for Formula II.

The invention further provides processes for preparing any of the compounds of the present invention through following at least a portion of the techniques recited in the experimental section.

The compounds of the invention are useful for the treatment, amelioration, or prevention of disorders characterized with enterovirus (e.g., EV-D68; EV-A71; CVB3) activity and/or expression (e.g., respiratory illness) (e.g., neuronal infection (e.g., acute flaccid myelitis (AFM))).

The compounds of the invention are useful for the treatment, amelioration, or prevention of disorders characterized with enterovirus (e.g., EV-D68; EV-A71; CVB3) activity and/or expression. In certain embodiments, the compounds can be used to treat, ameliorate, or prevent disorders characterized with enterovirus (e.g., EV-D68; EV-A71; CVB3) activity and/or expression such as, for example, respiratory illness and/or neuronal infection (e.g., acute flaccid myelitis (AFM)).

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents (e.g., antiviral agents).

The present disclosure further provides bifunctional compounds that function to recruit endogenous proteins to an E3 Ubiquitin Ligase for degradation, and methods of using the same. In particular, the present disclosure provides bifunctional or proteolysis targeting chimeric (PROTAC) compounds, which find utility as modulators of targeted ubiquitination of a variety of polypeptides and other proteins, which are then degraded and/or otherwise inhibited. An exemplary advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as any type of condition associated with enterovirus activity and/or expression.

In an additional aspect, the disclosure provides bifunctional or PROTAC compounds, which comprise an E3 Ubiquitin Ligase binding moiety (e.g., a ligand for an E3 Ubquitin Ligase or “ULM” group), and a moiety that binds a target protein (e.g., a protein/polypeptide targeting ligand or “PTM” group) (e.g., an enterovirus activity and/or expression inhibitor) such that the target protein/polypeptide is placed in proximity to the ubiquitin ligase to effect degradation (and inhibition) of that protein (e.g., inhibit enterovirus receptor activity and/or expression). In certain embodiments, the PTM is any of the compounds as described herein showing inhibitory activity against enterovirus activity and/or expression. In some embodiments, the ULM is a VHL, cereblon, mouse double minute 2 (MDM2), and/or inhibitor of apoptosis protein (IAP) E3 ligase binding moiety. For example, the structure of the bifunctional compound can be depicted as PTM-ULM.

The respective positions of the PTM and ULM moieties, as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as PTM-L-ULM, where PTM is a protein/polypeptide targeting moiety (e.g., any of the compounds as described herein showing inhibitory activity against enterovirus activity and/or expression), L is a linker, and ULM is a VHL, cereblon, MDM2, or IAP E3 ligase binding moiety binding moiety.

Such embodiments are not limited to a specific type of linker. In some embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical. In some embodiments, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

The ULM group and PTM group may be covalently linked to the linker group through any group which is appropriate and stable to the chemistry of the linker. In exemplary aspects of the present invention, the linker is independently covalently bonded to the ULM group and the PTM group in certain embodiments through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. In certain aspects where the PTM group is a ULM group, the target protein for degradation may be the ubiquitin ligase itself. In certain exemplary aspects, the linker may be linked to an optionally substituted alkyl, alkylene, alkene or alkyne group, an aryl group or a heterocyclic group on the ULM and/or PTM groups.

In certain embodiments, the compounds as described herein comprise multiple ULMs, multiple PTMs, multiple chemical linkers, or any combinations thereof.

In some embodiments, the present invention provides a method of ubiquitinating/degrading enterovirus receptor activity and/or expression in a cell comprising administering a bifunctional compound as described herein comprising an ULM and a PTM, in certain embodiments linked through a linker moiety, as otherwise described herein, wherein the ULM is coupled to the PTM and wherein the ULM recognizes a ubiquitin pathway protein and the PTM recognizes the target protein such that degradation of the target protein occurs when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

### BRIEF DESCRIPTION OF THE DRAWINGS

FIG. 1: Chemical structure of dibucaine. Three positions, 2, 4, and 6, were examined in the current SAR studies and were circled.

FIG. 2: Summary of SAR studies of dibucaine described in Example I.

FIG. 3A-C: Dibucaine and its analogs 10a, 12a, and 12c reduced the EV-D68 viral protein and RNA levels. (A) Dibucaine and its analogs inhibited EV-D68 replication. RD cells infected with EV-D68 strain US/KY/14-18953 (MOI=1) were fixed at 9 hpi and stained with anti-VP1 and DAPI for detecting VP1 and nucleus, respectively. (B and C) Dibucaine and its analogs reduced the EV-D68 viral protein and RNA levels. RD cells were infected with EV-D68 strain US/KY/14-18953 (MOI=1) with treatment of indicated compound. At 9 hpi, cells were harvested for viral protein quantification by western or viral RNA quantification by RT-qPCR. Asterisks indicate statistically significant difference in comparison with the DMSO control (one-way ANOVA analysis by Prism 5, ***P<0.001). The value is the mean of two independent experiments±standard deviation.

FIG. 4: Inhibition of sodium channels by dibucaine and lead compound 10a.

FIG. 5: Dibucaine and the optimized lead compounds did not inhibit influenza A and B viruses. Plaque assay was performed using the condition described previously (see, Wang, Y.; et al., J. Med. Chem. 2018, 61, 1074-1085). Dibucaine, 10a, 12a, and 12c were tested at 10 μM. Oseltamivir carboxylate was included as a positive control and was tested at 200 nM.

FIG. 6A-D: Binding of fluoxetine, guanidine, and 6aw to CVB3 2C protein using differential scanning fluorimetry. CVB3 (4.5 μM) 2C was incubated with DMSO (black), 100 μM fluoxetine (A), 1 mM guanidine (B), or different concentrations of 6aw (C), and the raw DSF data were plotted and curve fitting were performed using the Boltzmann sigmoidal equation in Prism (v5) software to determine the Tₘ and ΔTₘ (D).

### DETAILED DESCRIPTION OF THE INVENTION

The human enteroviruses (EVs) are a group of small non-enveloped single-stranded positive-sense RNA viruses that belong to the piconaviridae family (see, Van der Linden, L.; et al., Viruses 2015, 7, 4529-4562; Baggen, J.; et al., Nat. Rev. Micrbiol. 2018, 16, 368-381). Up to date, more than 110 serotypes of enteroviruses have been identified, among which the most notable ones are polioviruses, coxsackieviruses, EV-D68, EV-A71, and rhinoviruses (see, Glyn Stanway, T. H., Nick J. Knowles, Timo Hyypia. Molecular and biological basis of picornavirus taxonomy. In Molecular Biology of Picornavirus, Wimmer, B. L. S. a. E., Ed. ASM Press: Washington D.C., 2002; pp 17-24). Although poliovirus was nearly eradicated by immunization, no vaccines are available for the non-polio EVs except EV-A71 (see, Mao, Q.; et al., Emerg. Microbes Infect. 2016, 5, e75). The non-polio enteroviruses such as EV-D68 are of particular concern as they have been shown to be causative pathogens of numerous epidemics of moderate to severe respiratory illness (see, Tyler, K. L. N. Engl. J. Med. 2018, 379, 557-566; Holm-Hansen, C. C.; et al., Lancet Infect. Dis. 2016, 16, e64-e75; Midgley, C. M.; et al., Lancet Respir. Med. 2015, 3, 879-887; Oermann, C. M.; et al., Ann. Am. Thorac. Soc. 2015, 12, 775-781). Although infections in most cases are mild and self-limiting, EV-D68 infections can lead to serious and life-threatening neurological complications such as acute flaccid myelitis (AFM), most often in infants, young children, and immunocompromised individuals (see, Huang, H. I.; et al., Viruses 2015, 7, 6051-6066). Cases of EV-D68 infection were rarely reported before 2005 (see, Khetsuriani, N.; et al., MMWR Surveill Summ 2006, 55, 1-20), however, recent years have seen an increasing surge of EV-D68 infections around the world, especially in the United States (see, Holm-Hansen, C. C.; et al., Lancet Infect. Dis. 2016, 16, e64-e75; Abedi, G. R.; et al., MMWR Morb. Mortal. Wkly. Rep. 2018, 67, 515-518). EV-D68 attracted public attention when an outbreak spread to 49 states in the United States in 2014 that led to more than 1,100 reports of severe respiratory disease and more than 100 cases of acute flaccid myelitis (see, Holm-Hansen, C. C.; et al., Lancet Infect. Dis. 2016, 16, e64-e75). Since then, EV-D68 infection has been continuously reported.

EV-D68 is an atypical enterovirus and behaves more like a rhinovirus. For example, unlike other enteroviruses that replicate in the gastrointestinal tract at 37° C., EV-D68 is cold adapted and replicates more efficiently at 33° C. (see, Oberste, M. S.; et al., J. Gen. Virol. 2004, 85, 2577-2584). Individuals with pre-existing respiratory condition such as asthma or chronic obstructive pulmonary disease (COPD) are more likely to develop a severe infection (see, Midgley, C. M.; et al., Lancet Respir. Med. 2015, 3, 879-887). More alarmingly, there has been evidence from both animal studies and human patients that EV-D68 can spread to central nervous systems (CNS) such as spinal cord and cerebrospinal fluid through viremia, causing neurological complications such as AFM (see, Hurst, B. L.; et al., Virology 2019, 526, 146-154; Hixon, A. M.; et al., J. Infect. Dis. 2017, 216, 1245-1253; Zheng, H.; et al., J. Immunol. 2018, 201, 2557-2569; Hixon, A. M.; et al., PLoS Pathog. 2017, 13, e1006199; Greninger, A. L.; et al., Lancet Infect. Dis. 2015, 15, 671-682; Lafolie, J.; et al., Lancet Infect. Dis. 2018, 18, 1385-1396).

Due to its global medical and socioeconomic impact, EV-D68 is classified as a priority pathogen by NIAID. However, despite decades of research efforts, there is currently no vaccine nor small molecule antiviral exist for EV-D68 infection (see, Baggen, J.; et al., Nat. Rev. Micrbiol. 2018, 16, 368-381), and treatment is primarily limited to supportive care. To reduce the morbidity and mortality associated with EV-D68 virus infection, there is an urgent need to develop potent and selective small molecule antivirals. Towards this end, we are interested in developing EV-D68 antivirals by targeting the viral 2C protein.

The non-structural protein EV-D68 2C is a multi-functional protein. Genetic studies of 2C have revealed that it has functional roles in viral uncoating, RNA binding and replication, membrane rearrangement, encapsidation of the viral genome, and progeny viral assembly, all of which are essential for viral replication (see, Van der Linden, L.; et al., Viruses 2015, 7, 4529-4562; Baggen, J.; et al., Nat. Rev. Micrbiol. 2018, 16, 368-381). Therefore, EV-D68 2C appears to be an attractive target for anti-enterovirus drug development. Previous phenotypic screenings identified several small molecules such as pirlindole mesylate, fluoxetine, formoterol, dibucaine, and guanidine as EV-D68 antivirals (see, Smee, D. F.; et al., Antiviral Res. 2016, 131, 61-65; Ulferts, R.; et al., Antimicrob. Agents Chemother. 2016, 60, 2627-2638; Zuo, J.; et al., Antimicrob. Agents Chemother. 2015, 60, 1615-1626; Ulferts, R.; et al., Antimicrob. Agents Chemother. 2013, 57, 1952-1956). Resistance selection through serial viral passage experiments led to the identification of mutations in viral 2C protein that confer resistance to these compounds, suggesting 2C might be the potential drug target.

Experiments conducted during the course of developing embodiments for the present invention first independently verified the antiviral activity and cellular cytotoxicity of these reported 2C protein inhibitors. It was found that all these molecules had only moderate antiviral activity against EV-D68 with a low selectivity index (Table 1), consistent with previously reported results (see, Ulferts, R.; et al., Antimicrob. Agents Chemother. 2016, 60, 2627-2638; Zuo, J.; et al., Antimicrob. Agents Chemother. 2015, 60, 1615-1626). Next, experiments were conducted that chose dibucaine as a starting hit compound for structure-activity relationship (SAR) studies due to its modular synthetic accessibility. The aim was to optimize the potency and selectivity index of dibucaine through SAR studies. In total sixty quinoline analogues have been synthesized and tested against EV-D68 (US/KY/14-18953) virus in the primary viral cytopathic effect (CPE) assay. Three compounds (10a, 12a, and 12c) were identified to have significantly improved antiviral activity with EC₅₀ values in the submicromolar range and a selectivity index over 180. Experiments were conducted that further profiled the broad range antiviral activity of these three compounds against additional four contemporary human EV-D68 viruses. It was found that these three compounds all had potent antiviral activity and a high selectivity index. These three lead compounds also potently inhibited EV-D68 infection in neuronal cells such as A172 and SH-SYSY cells, suggesting they can be further developed to treat both respiratory and neuronal infections. The mechanism of action of these three compounds was studied by RT-qPCR, western blot, and immunofluorescence imaging. These compounds were found to inhibit viral RNA and protein synthesis, which agrees with the inhibition of viral 2C protein.

TABLE 1
Literature reported enterovirus 2C protein inhibitors.

| | EV-D68 | | |
|---|---|---|---|
| | (US/KY/14) | RD cells | |
| Structure | EC₅₀ (μM)^{a} | CC₅₀ (μM)^{b} | SI |
| 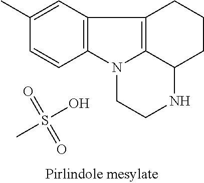 | 8.4 ± 1.1 | 19.0 ± 1.6 | 2.3 |
| 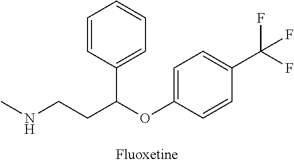 [SMILES: CNCCC(Oc1ccc(C(F)(F)F)cc1)c1ccccc1] | 1.0 ± 0.1 | 11.9 ± 4.9 | 11.9 |
| 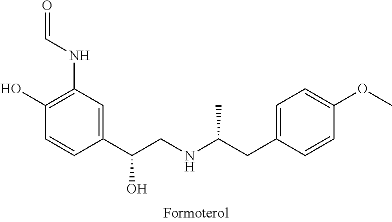 [SMILES: COc1ccc(C[C@@H](C)NC[C@H](O)c2ccc(O)c(NC=O)c2)cc1] | 24.7 ± 1.1 | 146.3 ± 27.8 | 5.9 |
| 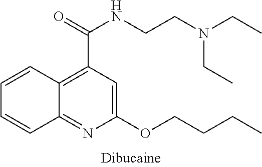 [SMILES: CCCCOc1cc(C(=O)NCCN(CC)CC)c2ccccc2n1] | 5.3 ± 1.2 | 56.6 ± 28.7 | 10.7 |
| 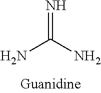 | 219.4 ± 15.0 | >300.0 | >1.4 |

^{a}Antiviral efficacy was determined in the CPE assay with EV-D68 US/KY/14-18953 virus and RD cells.
^{b}Cytotoxicity was determined using the neutral red method. The results are the mean ± standard deviation of three repeats.
SI = selectivity index (CC₅₀/EC₅₀).

There is no specific treatment for non-polio enterovirus infection. People with mild illness caused by non-polio enterovirus infection typically only need to treat their symptoms. This includes drinking enough water to stay hydrated and taking over-the-counter cold medications as needed. Most people recover completely. However, some illnesses caused by non-polio enteroviruses can be severe enough to require hospitalization.

Experiments conducted during the course of developing embodiments for the invention conducted structure-activity relationship studies and showed that chemical modification of dibucaine increased its potency against contemporary strains of EV-D68. However, their broad-spectrum antiviral activity against EV-A71 and coxsackievirus B3 virus remained to be profiled. Additional experiments were conducted that focused on optimizing the broad-spectrum antiviral activity and in vitro microsomal stability of quinoline analogs through structure-activity relationship (SAR) studies and structure-property (SPR) relationship studies. In addition, thermal shift binding assay was applied to quantify the direct binding between quinoline analogs and the viral 2C protein. Overall, such experiments revealed several promising quinoline analogs that had potent and broad-spectrum antiviral activity against EV-D68, EV-A71 and CVB3.

Accordingly, the present invention relates to a new class of small-molecule compounds having a quinoline (or similar) structure which function as enterovirus inhibitors, and their use as therapeutics for the treatment of conditions characterized with enterovirus activity (e.g., respiratory infections, neurological conditions).

In a particular embodiment, quinoline (or similar) compounds encompassed within Formulas I is provided:

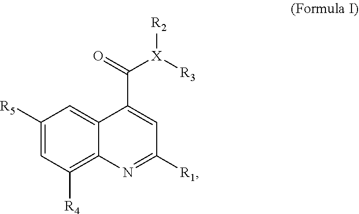

including pharmaceutically acceptable salts, solvates, and/or prodrugs thereof.

In some embodiments, X is either O or N.

Formula I is not limited to a particular chemical moiety for X, R1, R2, R3, R4 and R5. In some embodiments, the particular chemical moiety for X, R1, R2, R3, R4 and R5 independently include any chemical moiety that permits the resulting compound to inhibit enterovirus (e.g., non-polio enterovirus) (e.g., EV-D68, EV-A71, CVB3) activity and/or expression.

In some embodiments, R1 is selected from hydrogen,

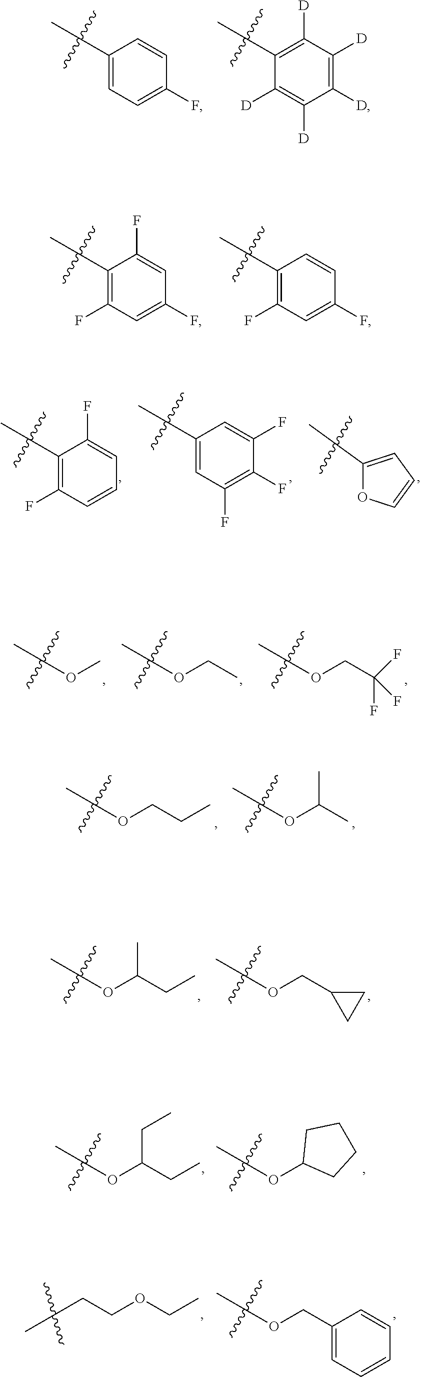

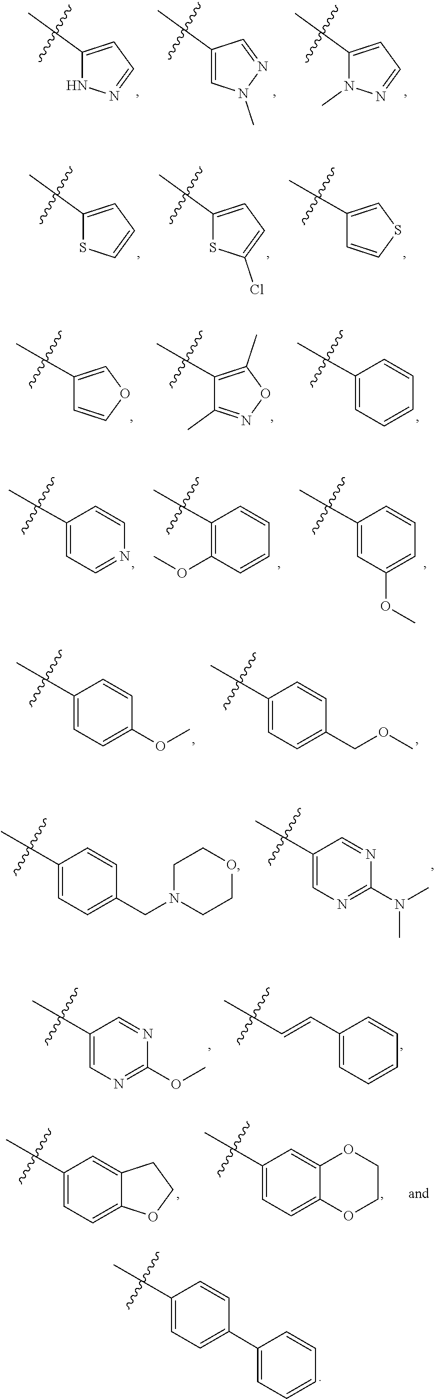

In some embodiments, R2 and R3 are independently selected from the group consisting of hydrogen,

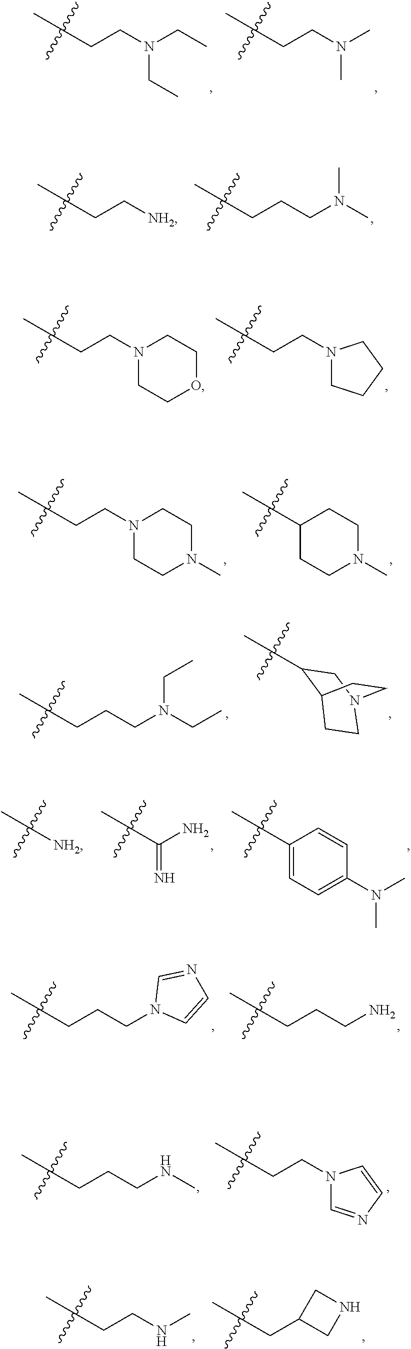

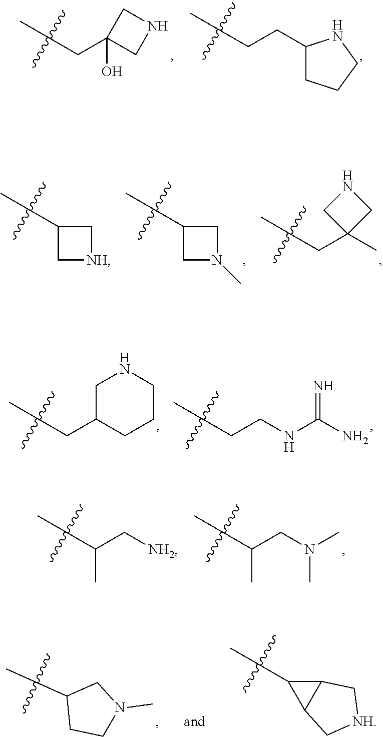

In some embodiments, R2 and R3 combine such that

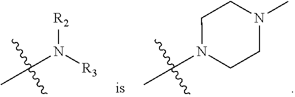

In some embodiments, R4 is selected from hydrogen and halogen (e.g., Chlorine).

In some embodiments, R5 is selected from hydrogen, halogen (e.g., fluorine, chlorine), and methoxy.

In some embodiments, compounds shown in Tables 2 (not including dibucaine), 3, 4, 5, 6 and 9 are contemplated for Formula I.

In a particular embodiment, quinoline (or similar) compounds encompassed within Formulas II is provided:

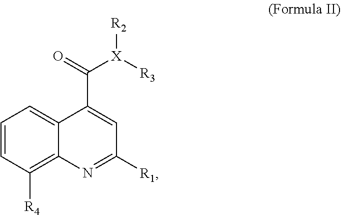

including pharmaceutically acceptable salts, solvates, and/or prodrugs thereof.

Formula II is not limited to a particular chemical moiety for X, R1, R2, R3 and R4. In some embodiments, the particular chemical moiety for X, R1, R2, R3 and R4 independently include any chemical moiety that permits the resulting compound to inhibit enterovirus (e.g., non-polio enterovirus) (e.g., EV-D68, EV-A71, CVB3) activity and/or expression.

In some embodiments, X is either O or N.

In some embodiments, R1 is selected from

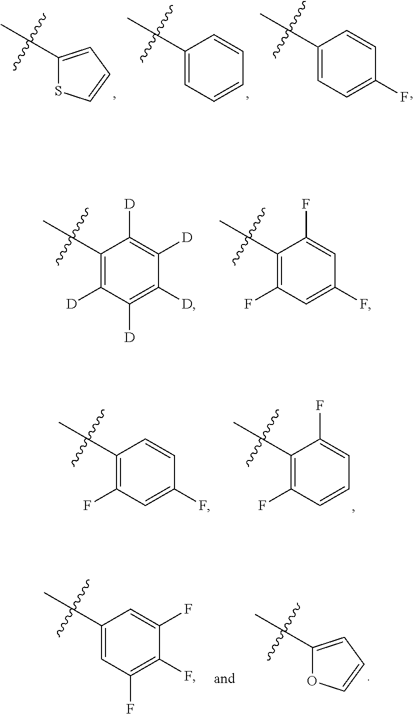

In some embodiments, R2 and R3 are independently selected from hydrogen,

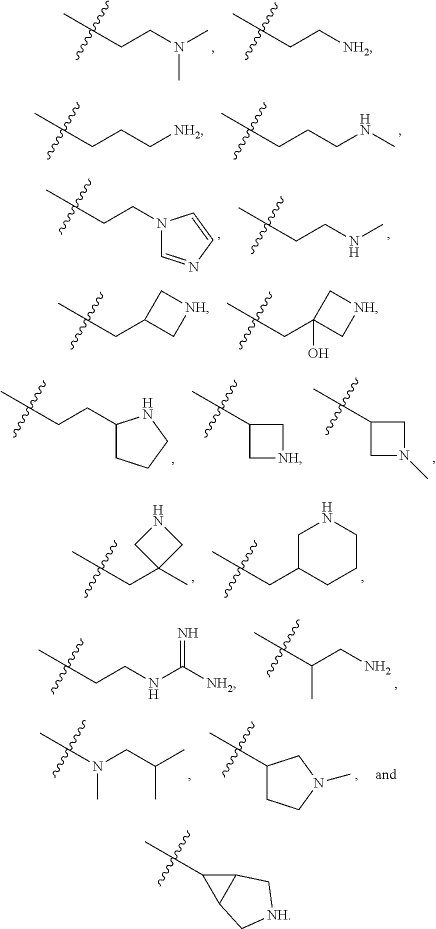

In some embodiments, R4 is selected from hydrogen and halogen (e.g., Chlorine). In some embodiments, compounds shown in Table 9 are contemplated for Formula II.

An important aspect of the present invention is that compounds of the invention are capable of inhibiting the activity and/or expression of a variety of enteroviruses including, but not limited to non-polio enteroviruses (e.g., EV-D68; EV-A71; CVB3). Therefore, it is contemplated that these compounds can be used in the treatment of conductions characterized with enterovirus infection. Indeed, these enterovirus inhibitors can be used to treat, ameliorate, or prevent such conditions characterized with enterovirus activity and/or expression.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, any type of condition characterized with enterovirus (e.g., EV-D68; EV-A71; CVB3) infection (e.g., respiratory conditions, neuronal conditions (e.g., acute flaccid myelitis (AFM))).

Some embodiments of the present invention provide methods for administering an effective amount of a compound of the invention and at least one additional therapeutic agent (including, but not limited to, any type of antiviral agent.

In some embodiments of the present invention, a compound of the invention and one or more antiviral agents are administered to an animal under one or more of the following conditions: at different periodicities, at different durations, at different concentrations, by different administration routes, etc. In some embodiments, the compound is administered prior to the antiviral agent, e.g., 0.5, 1, 2, 3, 4, 5, 10, 12, or 18 hours, 1, 2, 3, 4, 5, or 6 days, or 1, 2, 3, or 4 weeks prior to the administration of the antiviral agent. In some embodiments, the compound is administered after the antiviral agent, e.g., 0.5, 1, 2, 3, 4, 5, 10, 12, or 18 hours, 1, 2, 3, 4, 5, or 6 days, or 1, 2, 3, or 4 weeks after the administration of the antiviral agent. In some embodiments, the compound and the antiviral agent are administered concurrently but on different schedules, e.g., the compound is administered daily while the antiviral agent is administered once a week, once every two weeks, once every three weeks, or once every four weeks. In other embodiments, the compound is administered once a week while the antiviral agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example describes the discovery of quinoline analogs as potent antivirals against enterovirus D68 (EV-D68).

### Chemistry

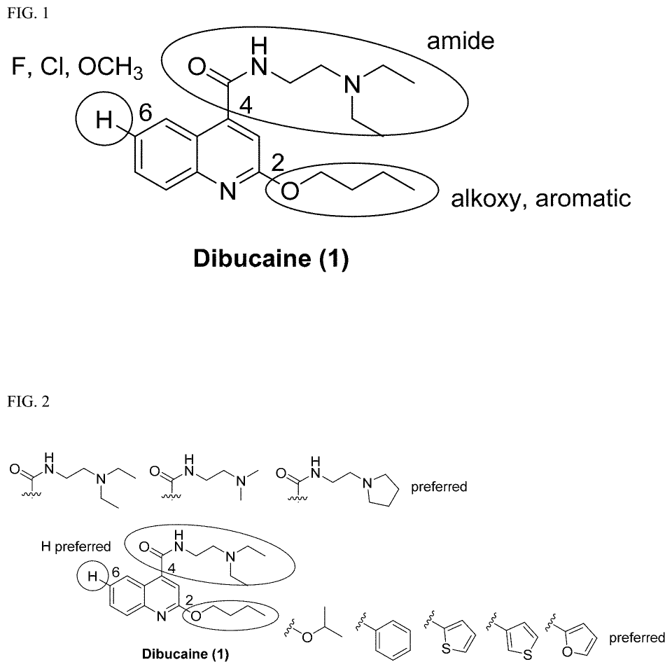

Three positions of dibucaine were varied in a structure activity relationship (SAR) study: the 2-position substituent of dibucaine was substituted with either an alkoxy (O—R₁) or an aromatic group (R2); the 4-position was substituted with different amides; and the 6-position was substituted with either F, Cl, or methoxy group. A stepwise iterative optimization strategy was implemented (see, FIG. 1). First, the 4-position substitution was fixed while the 2-position substitution was optimized. Second, the 2-position substitution was fixed while the 4-position substitution was optimized. Third, optimal substitutions from both the 2- and 4-positions were merged. Finally, the 6-position was diversified with the most potent analogs.

The synthesis of quinoline analogs was designed to allow for expeditious late-stage diversification. For example, for varying the 2-position substitution, intermediate 4 was first synthesized by amide coupling. Next, two methods were developed to install alkoxy substitution at the 2-position (Scheme 1, synthesis route 1). Method 1 used sodium metal and excess alcohol, which served as both the reagent and the solvent. This worked for alcohols with a low boiling point such that they can be easily removed during the work up procedure. For alcohols with a high boiling point, method 2 was developed which used DMF as the solvent and two equivalents of sodium hydride and the alcohol. To introduce aromatic substitutions at the 2-position, Suzuki-Miyaura cross coupling was employed using microwave heating (Scheme 1, synthesis route 2). Once the optimal substations at the 2-position were identified, the next step was to optimize the 4-position amide substitution. For this, quinoline 4-carboxylic acids 7, 9, and 11 with optimal 2-position substitutions (isopropoxyl, phenyl, and thienyl) were synthesized. Next, amide coupling was applied to introduce different amines (Scheme 1, synthesis routes 3 & 4). Finally, for analogs with substitutions at the 6-position of quinoline, two synthesis strategies were developed (Scheme 1, synthesis routes 5) (see, Baragana, B.; et al., J. Med. Chem. 2016, 59, 9672-9685). The first strategy involved Pfitzinger reaction with 5-chloroisatin or 5-methoxylisatin 13 and acetophenone using potassium hydroxide as a base. Coupling of 14 with different amines gave the final product 15. The second strategy involved the reaction of 5-fluoroisatin with malonic acid under refluxing condition in acetic acid to give intermediate 17. Next, a one pot chlorination and amide coupling was achieved by first forming the 2-chloroquinoline-4-carbonyl chloride intermediate through reacting intermediate 17 with thionyl chloride in the presence of catalytic amount of DMF, then different amines were added to convert the acid chloride intermediate to the intermediate 18. Finally, Suzuki-Miyaura cross-coupling was applied to install the 2-position substitution to give the final product 19.

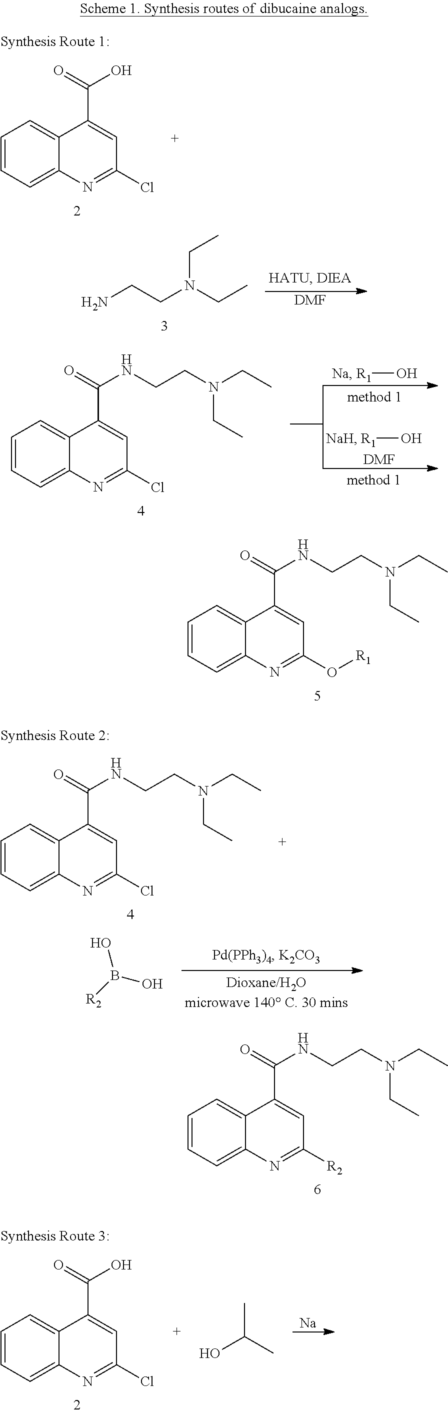

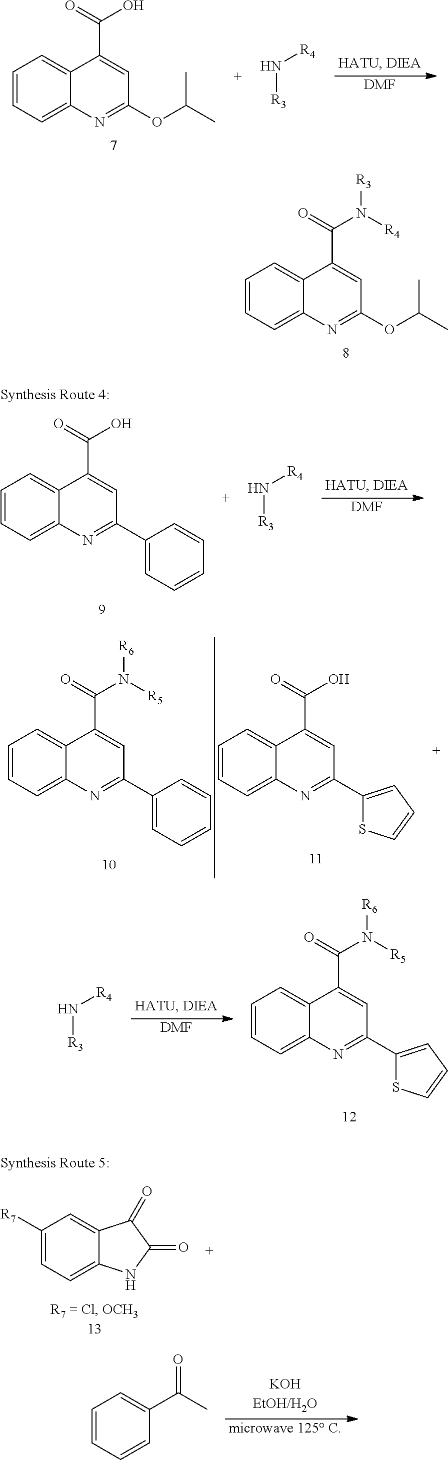

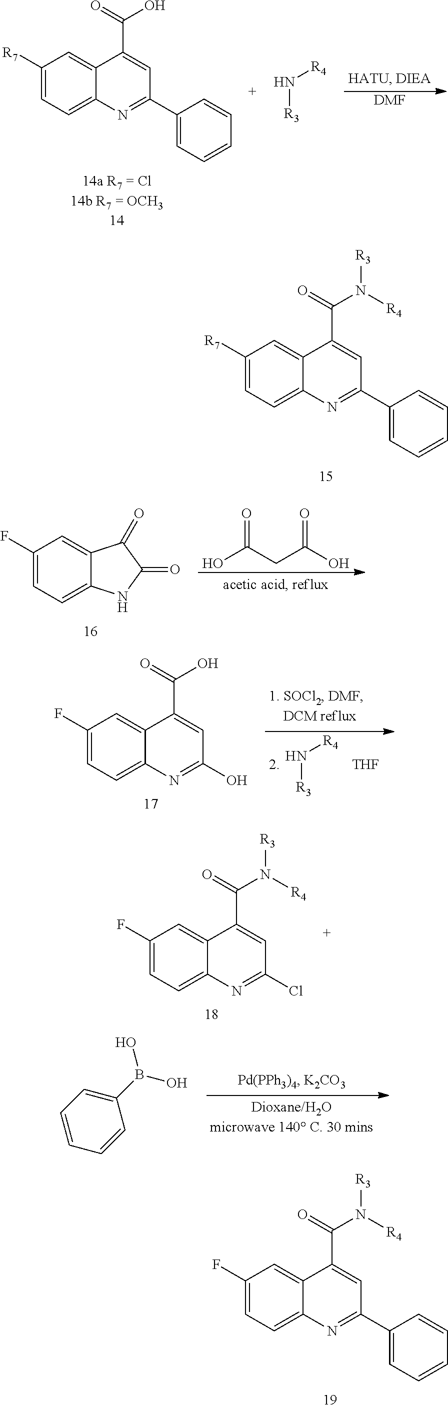

### Structure Activity Relationship (SAR) Studies

As an initial screening, all compounds were tested for antiviral activity and cellular cytotoxicity in standard viral CPE assay and neutral red cell viability assay, respectively, with the human rhabdomyosarcoma (RD) cells. The CPE assay involved infecting RD cells with the EV-D68 virus (US/KY/14-18953) and monitoring cell viability after 3 days of incubation with and without compound treatment. Dibucaine was included as a positive control. For compound 5 series with different alkoxy substitutions at the 2-position (Table 2), compound 5e with 2-isopropoxy substitution was found to have the highest potency and selectivity (EC₅₀=2.5±0.5 μM, CC₅₀=111.2±15.4 μM, SI=44.5). Compound 5k also had improved potency and selectivity compared to dibucaine. Other compounds (5a, 5b, 5c, 5d, 5f, 5g, 5h, 5i, and 5j) either had reduced antiviral activity (5a, 5b, 5g, 5h, 5i, 5j) or reduced selectivity index (5c, 5d, 5f) compared to dibucaine. From these results, it can be concluded that isopropoxy is one of the optimal substitutions at the 2-position of quinoline. Therefore, compound 5e was chosen as a reference compound for following optimization.

TABLE 2
SAR study of the 2-position R₁ substituent: alkoxy
5

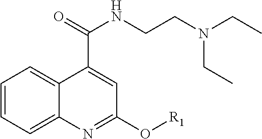

| Com- | | | | |
|---|---|---|---|---|
| pound | | EC₅₀ | CC₅₀ | |
| ID | R₁ | (μM)^{a} | (μM)^{b} | SI |
| 5a | 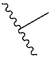 | 52.5 ± 7.1 | >250.0 | >4.8 |
| 5b | 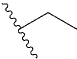 [SMILES: *CC] | 5.9 ± 3.2 | 164.9 ± 57.6 | 27.9 |
| 5c | 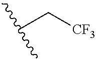 [SMILES: *CC(F)(F)F] | 3.5 ± 0.4 | 33.1 ± 1.8 | 9.5 |
| 5d | 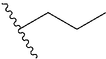 [SMILES: *CCC] | 4.9 ± 2.0 | 46.7 ± 14.7 | 9.5 |
| 5e | 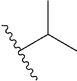 [SMILES: *C(C)C] | 2.5 ± 0.5 | 111.2 ± 15.4 | 44.5 |
| Dibu- caine 1 | 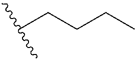 [SMILES: *CCCC] | 5.3 ± 1.2 | 56.6 ± 28.7 | 10.7 |
| 5f | 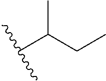 [SMILES: *C(C)CC] | 2.8 ± 1.3 | 15.7 ± 1.1 | 5.6 |
| 5g | 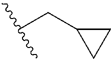 [SMILES: *CC1CC1] | 7.1 ± 5.0 | 38.5 ± 16.8 | 5.4 |
| 5h | 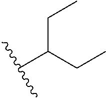 [SMILES: *C(CC)CC] | >10 | 9.5 ± 1.1 | N.A.^{c} |
| 5i | 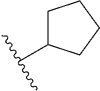 [SMILES: *C1CCCC1] | 6.8 ± 0.5 | 11.7 ± 1.2 | 1.7 |
| 5j | 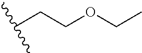 [SMILES: *CCOCC] | 12.2 ± 1.1 | 143.7 ± 40.5 | 11.8 |
| 5k | 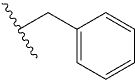 [SMILES: *Cc1ccccc1] | 1.1 ± 0.1 | 13.5 ± 2.9 | 12.3 |

^{a}Antiviral efficacy was determined in the CPE assay with EV-D68 US/KY/14-18953 virus and RD cells.
^{b}Cytotoxicity was determined using the neutral red method.
^{c}N.A. = not applicable.
The results are the mean ± standard deviation of three repeats.
SI = selectivity index (CC₅₀/EC₅₀).

For compound 6 series with aromatic substitutions at the 2-position (Table 3), compounds 6d, 6f, 6g, and 6j had improved antiviral activity and selectivity index compared to compound 5e. Compounds 6e, 6l, 6m, 6r, and 6s had improved antiviral activity compared to 5e, however they were compromised by a lower selectivity index. Other compounds (6a, 6b, 6c, 6h, 6i, 6k, 6n, 6o, 6p, 6q, 6t, and 6u) in this series were less active than 5e and were not further explored. These results suggested unsubstituted thienyl, furanyl, and benzyl are optimal substitutions at the 2-position of quinoline.

TABLE 3
SAR study of the 2-position R₂ substituent: Aromatic Groups
6

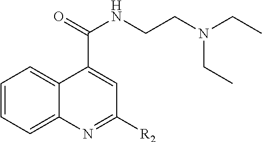

| Com- | | | | |
|---|---|---|---|---|
| pound | | EC₅₀ | CC₅₀ | |
| ID | R₂ | (μM)^{a} | (μM)^{b} | SI |
| 5e | 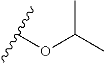 [SMILES: *OC(C)C] | 2.5 ± 0.5 | 111.2 ± 15.4 | 44.5 |
| 6a | 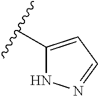 [SMILES: *c1ccn[nH]1] | 19.4 ± 1.2 | 132.3 ± 53.3 | 6.8 |
| 6b | 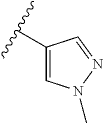 [SMILES: *c1cnn(C)c1] | >100.0 | 250.7 ± 28.3 | N.A.^{c} |
| 6c | 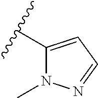 [SMILES: *c1ccnn1C] | 36.3 ± 1.6 | 132.6 ± 28.7 | 3.7 |
| 6d | 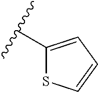 [SMILES: *c1cccs1] | 0.6 ± 0.5 | 37.4 ± 6.3 | 62.3 |
| 6e | 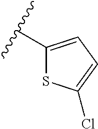 [SMILES: *c1ccc(Cl)s1] | 0.8 ± 0.6 | 6.1 ± 2.2 | 7.6 |
| 6f | 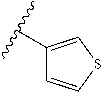 [SMILES: *c1ccsc1] | 1.0 ± 0.4 | 46.9 ± 5.2 | 46.9 |
| 6g | 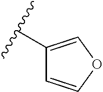 [SMILES: *c1ccoc1] | 1.5 ± 0.9 | 122.5 ± 30.8 | 81.7 |
| 6h | 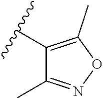 [SMILES: *c1c(C)noc1C] | >50.0 | 128.6 ± 67.2 | N.A.^{c} |
| 6i | 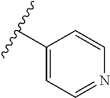 [SMILES: *c1ccncc1] | 6.6 ± 3.3 | 173.5 ± 55.6 | 26.3 |
| 6j | 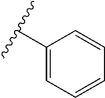 [SMILES: *c1ccccc1] | 0.8 ± 0.3 | 45.1 ± 13.7 | 56.4 |
| 6k | 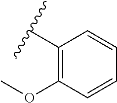 [SMILES: *c1ccccc1OC] | 13.9 ± 8.1 | 75.9 ± 6.4 | 5.5 |
| 6l | 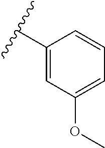 [SMILES: *c1cccc(OC)c1] | 1.6 ± 0.8 | 36.5 ± 3.1 | 22.8 |
| 6m | 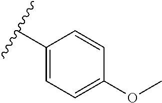 [SMILES: *c1ccc(OC)cc1] | 2.4 ± 0.2 | 31.6 ± 4.2 | 13.2 |
| 6n | 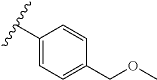 [SMILES: *c1ccc(COC)cc1] | 17.4 ± 8.9 | 46.6 ± 2.4 | 2.7 |
| 6o | 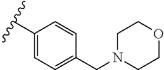 [SMILES: *c1ccc(CN2CCOCC2)cc1] | >80.0 | 79.2 ± 8.0 | N.A.^{c} |
| 6p | 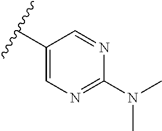 [SMILES: *c1cnc(N(C)C)nc1] | 24.6 ± 2.2 | 74.2 ± 7.0 | 3.0 |
| 6q | 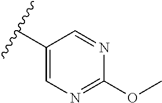 [SMILES: *c1cnc(OC)nc1] | 87.6 ± 3.1 | 173.5 ± 20.3 | 2.0 |
| 6r | 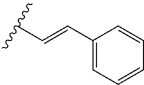 [SMILES: */C=C/c1ccccc1] | 0.8 ± 0.2 | 11.5 ± 6.7 | 14.4 |
| 6s | 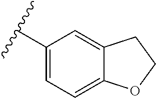 [SMILES: *c1ccc2c(c1)CCO2] | 1.6 ± 0.1 | 29.5 ± 1.6 | 18.4 |
| 6t | 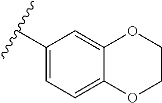 [SMILES: *c1ccc2c(c1)OCCO2] | 9.0 ± 1.3 | 49.9 ± 13.8 | 5.5 |
| 6u | 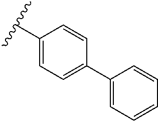 [SMILES: *c1ccc(-c2ccccc2)cc1] | >10.0 | 9.8 ± 0.5 | N.A.^{c} |

^{a}Antiviral efficacy was determined in the CPE assay with EV-D68 US/KY/14-18953 virus and RD cells.
^{b}Cytotoxicity was determined using the neutral red method.
^{c}N.A. = not applicable.
The results are the mean ± standard deviation of three repeats.
SI = selectivity index (CC₅₀/EC₅₀).

Once the 2-position substitution was optimized, the next step was to optimize the 4-position amide. For compound 8 series with 2-position substitution being isopropoxyl (Table 4), compounds 8a and 8e had improved antiviral activity compared with 5e, however they had a lower selectivity index. All other compounds (8b, 8c, 8d, 8f, 8g, and 8h) had reduced antiviral activity. For compound 10 and 12 series with 2-position being benzyl and thienyl, respectively (Table 5), compound 10a had significantly improved antiviral activity and selectivity index (EC₅₀=0.4±0.2 μM, CC₅₀=73.7±19.1 μM, SI=184.3). Similarly, compounds 12a and 12c were also found to be highly potent EV-D68 antivirals (EC₅₀<0.1 μM) with a high selectivity index (SI>600).

TABLE 4
SAR study of the 4-position amide substituent
8

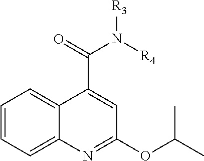

| Compound ID | 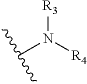 | EC₅₀ (μM)^{a} | CC₅₀ (μM)^{b} | SI |
|---|---|---|---|---|
| 5e | 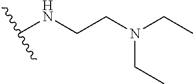 [SMILES: *NCCN(CC)CC] | 2.5 ± 0.5 | 111.2 ± 15.4 | 44.5 |
| 8a | 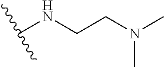 [SMILES: *NCCN(C)C] | 1.7 ± 1.2 | 38.5 ± 1.5 | 22.6 |
| 8b | 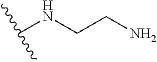 [SMILES: *NCCN] | 3.5 ± 0.3 | 148.8 ± 15.3 | 42.5 |
| 8c | 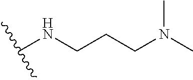 [SMILES: *NCCCN(C)C] | 11.4 ± 0.7 | 121.7 ± 39.2 | 10.7 |
| 8d | 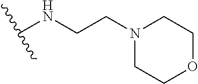 [SMILES: *NCCN1CCOCC1] | 17.5 ± 10.2 | >250.0 | >14.3 |
| 8e | 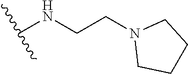 [SMILES: *NCCN1CCCC1] | 1.7 ± 1.2 | 36.9 ± 20.1 | 21.7 |
| 8f | 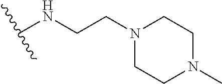 [SMILES: *NCCN1CCN(C)CC1] | 25.9 ± 2.1 | 88.8 ± 21.5 | 3.4 |
| 8g | 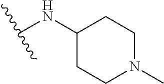 [SMILES: *NC1CCN(C)CC1] | 17.8 ± 1.1 | 100.7 ± 8.8 | 5.7 |
| 8h | 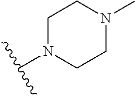 [SMILES: *N1CCN(C)CC1] | >100.0 | >300.0 | N.A.^{c} |

^{a}Antiviral efficacy was determined in the CPE assay with EV-D68 US/KY/14-18953 virus and RD cells.
^{b}Cytotoxicity was determined using the neutral red method.
^{c}N.A. = not applicable. The results are the mean ± standard deviation of three repeats.
SI = selectivity index (CC₅₀/EC₅₀).

TABLE 5
SAR study of the 4-position amide substituent
10 & 12

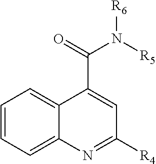

| Compound ID | 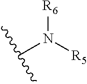 | R₄ | EC₅₀ (μM) | CC₅₀ (μM) | SI |
|---|---|---|---|---|---|
| 6j | 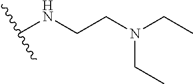 [SMILES: *NCCN(CC)CC] | 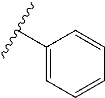 [SMILES: *c1ccccc1] | 0.8 ± 0.3 | 45.1 ± 13.7 | 56.4 |
| 10a | 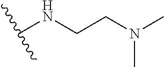 [SMILES: *NCCN(C)C] | 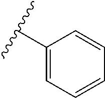 [SMILES: *c1ccccc1] | 0.4 ± 0.2 | 73.7 ± 19.1 | 184.3 |
| 10b | 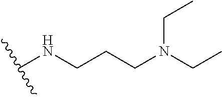 [SMILES: *NCCCN(CC)CC] | 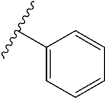 [SMILES: *c1ccccc1] | 9.6 ± 6.7 | 45.4 ± 14.0 | 4.7 |
| 10c | 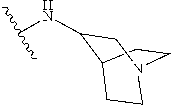 [SMILES: *NC1CN2CCC1CC2] | 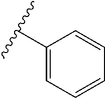 [SMILES: *c1ccccc1] | 3.5 ± 0.3 | 46.5 ± 14.7 | 13.3 |
| 10d | 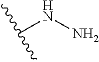 [SMILES: *NN] | 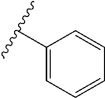 [SMILES: *c1ccccc1] | 100.1 ± 8.5 | >300.0 | >3 |
| 10e | 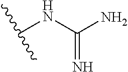 [SMILES: *NC(=N)N] | 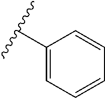 [SMILES: *c1ccccc1] | 34.7 ± 1.4 | >250 | >7.2 |
| 10f | 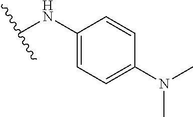 [SMILES: *Nc1ccc(N(C)C)cc1] | 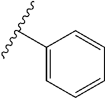 [SMILES: *c1ccccc1] | >100.0 | >350.0 | >3.5 |
| 10g | 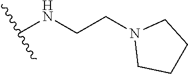 [SMILES: *NCCN1CCCC1] | 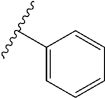 [SMILES: *c1ccccc1] | 0.4 ± 0.1 | 46.7 ± 8.3 | 116.8 |
| 10h | 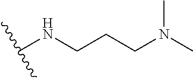 [SMILES: *NCCCN(C)C] | 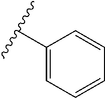 [SMILES: *c1ccccc1] | 3.5 ± 0.6 | 44.9 ± 17.7 | 12.8 |
| 12a | 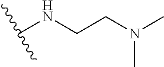 [SMILES: *NCCN(C)C] | 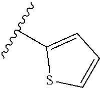 [SMILES: *c1cccs1] | 0.04 ± 0.01 | 42.3 ± 14.3 | 1057.5 |
| 12b | 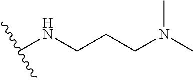 [SMILES: *NCCCN(C)C] | 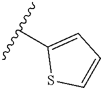 [SMILES: *c1cccs1] | 0.7 ± 0.3 | 40.5 ± 14.0 | 57.9 |
| 12c | 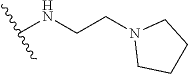 [SMILES: *NCCN1CCCC1] | 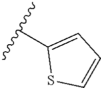 [SMILES: *c1cccs1] | 0.06 ± 0.03 | 40.0 ± 13.1 | 666.7 |
| 12d | 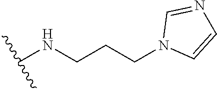 [SMILES: *NCCCn1ccnc1] | 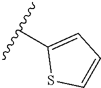 [SMILES: *c1cccs1] | 8.9 ± 5.8 | 55.7 ± 3.5 | 6.3 |
| 12e | 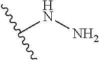 [SMILES: *NN] | 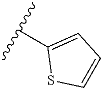 [SMILES: *c1cccs1] | 11.9 ± 1.3 | >100.0 | >8.4 |

^{a}Antiviral efficacy was determined in the CPE assay with EV-D68 US/KY/14-18953 virus and RD cells.
^{b}Cytotoxicity was determined using the neutral red method. The results are the mean ± standard deviation of three repeats.
SI = selectivity index (CC₅₀/EC₅₀).

For compounds 19 and 15 with substitutions at the 6-position (Table 6), none of them had improved potency and selectivity index compared to compound 10a, suggesting the 6-position of quinoline preferred to be unsubstituted.

TABLE 6
SAR study of the R₇ substituent
15 & 19

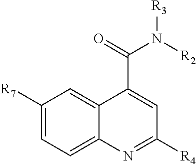

| Compound ID | 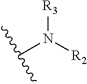 | R₄ | R₇ | EC₅₀ (μm) | CC₅₀ (μm) | SI |
|---|---|---|---|---|---|---|
| 10a | 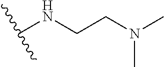 [SMILES: *NCCN(C)C] | 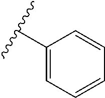 [SMILES: *c1ccccc1] | 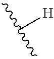 [SMILES: *[H]] | 0.4 ± 0.2 | 73.7 ± 19.1 | 184.3 |
| 19a | 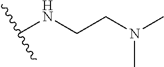 [SMILES: *NCCN(C)C] | 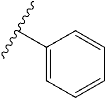 [SMILES: *c1ccccc1] | 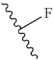 [SMILES: *F] | 0.6 ± 0.3 | 38.8 ± 19.0 | 64.7 |
| 19b | 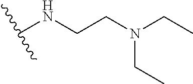 [SMILES: *NCCN(CC)CC] | 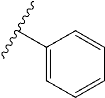 [SMILES: *c1ccccc1] | 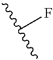 [SMILES: *F] | 1.3 ± 0.3 | 24.9 ± 1.5 | 19.2 |
| 15a | 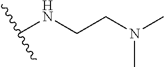 [SMILES: *NCCN(C)C] | 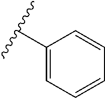 [SMILES: *c1ccccc1] | 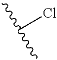 [SMILES: *Cl] | 1.2 ± 0.2 | 14.3 ± 0.8 | 11.9 |
| 15b | 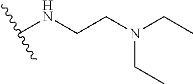 [SMILES: *NCCN(CC)CC] | 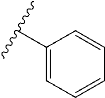 [SMILES: *c1ccccc1] | 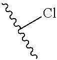 [SMILES: *Cl] | 1.9 ± 0.1 | 13.5 ± 8.1 | 7.1 |
| 15c | 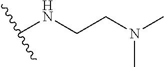 [SMILES: *NCCN(C)C] | 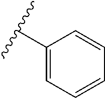 [SMILES: *c1ccccc1] | 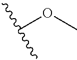 [SMILES: *OC] | 8.1 ± 0.6 | 31.8 ± 1.3 | 3.9 |
| 15d | 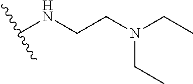 [SMILES: *NCCN(CC)CC] | 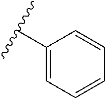 [SMILES: *c1ccccc1] | 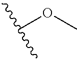 [SMILES: *OC] | 12.3 ± 1.1 | 27.5 ± 7.3 | 2.2 |

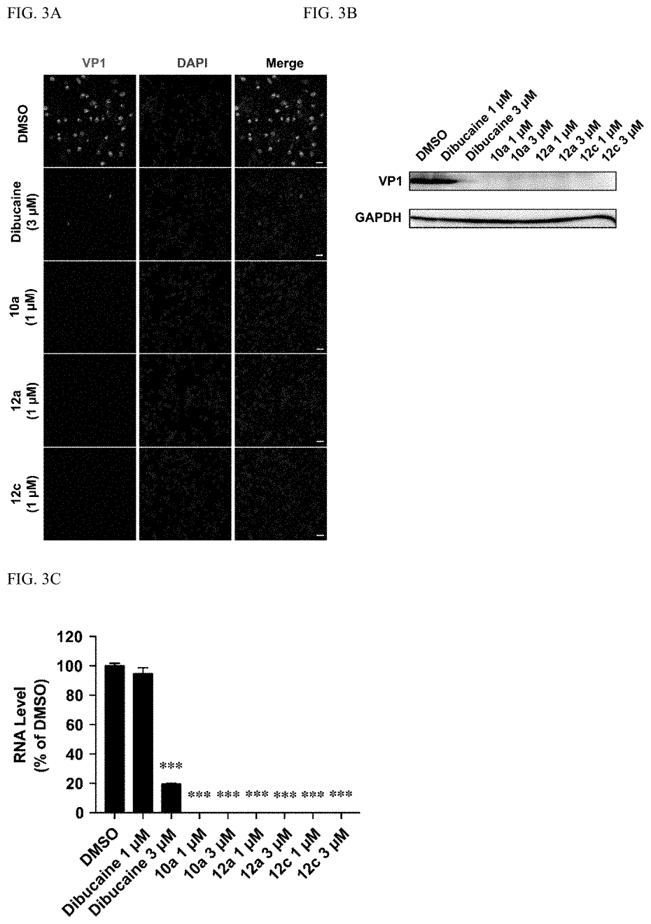

Overall this SAR study suggests that the optimal substitutions at the 2-position were isopropoxyl, thienyl, furanyl, and benzyl (FIG. 2); the optimal substitutions at the 4-position were N-(2-(diethylamino)ethyl)amide, N-(2-(dimethylamino)ethyl)amide, and N-(2-(pyrrolidin-1-yl)ethyl)amide; the 6-position preferred to be un-substituted.

### Broad Range Antiviral Activity

Given the potent antiviral activity and the high selectivity index of lead compounds 10a, 12a, and 12c in inhibiting the EV-D68 US/KY/14-18953 strain, experiments were conducted that were interested in exploring their broad range antiviral activity against other contemporary EV-D68 strains. For this, these three compounds were tested against four additional human EV-D68 strains (Table 7). It was found that compounds 10a, 12a, and 12c had similarly potent antiviral activity and high selectivity index against all five EV-D68 strains tested, corroborating that 2C protein is a conserved and high-profile antiviral drug target. The three lead compounds 10a, 12a, and 12c were also well tolerated in two additional cell lines A549 and HeLa (Table 7). Furthermore, as EV-D68 also infects CNS such as spinal cord and brain, we were intrigued to figure out whether these three lead compounds could also inhibit EV-D68 virus replication in the neuronal cells. For this purpose, we infected the human brain glioblastoma A172 cells or the neuroblastoma cell line SH-SYSY with the EV-D68 virus and quantified viral replication in CPE assay with and without compound treatment (Table 8). Encouragingly, all three compounds had potent antiviral activity against EV-D68 virus replication in neuronal cell lines (EC₅₀=0.01 to 0.4 μM). Notably, they were less cytotoxic to A172 and SH-SYSY cells than the RD cells, resulting in a higher selectivity index. In conclusion, the three lead compounds identified from the SAR studies, 10a, 12a, and 12c inhibits multiple EV-D68 strains with high potency and selectivity index in both muscle cells (RD) and neuronal cells (A172 and SH-SYSY).

TABLE 7
Antiviral activity of potent leads against different serotypes of EV-D68 in RD cells
and cytotoxicity of lead compounds in different cell lines

| | Antiviral activity and cytotoxicity in RD cells | | | | | cytotoxicity | |
|---|---|---|---|---|---|---|---|
| | US/KY/14- | US/MO/14- | US/MO/14- | US/IL/14- | UA/IL/14- | A549 | HeLa |
| | 18953 | 18947 | 18949 | 18956 | 18952 | (μM) | (μM) |
| Compound ID | (μM) | (μM) | (μM) | (μM) | (μM) | | |
| 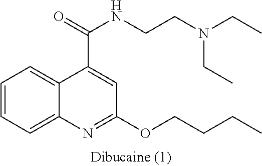 [SMILES: CCCCOc1cc(C(=O)NCCN(CC)CC)c2ccccc2n1] | EC₅₀ = 5.3 ± 1.2 CC₅₀ = 56.6 ± 28.7 SI = 10.7 | EC₅₀ = 3.5 ± 2.3 CC₅₀ = 56.6 ± 28.7 SI = 16.2 | EC₅₀ = 2.9 ± 1.8 CC₅₀ = 56.6 ± 28.7 SI = 19.5 | EC₅₀ = 1.8 CC₅₀ = 56.6 ± 28.7 SI = 31.4 | EC₅₀ = 2.0 ± 0.7 CC₅₀ = 56.6 ± 28.7 SI = 28.3 | CC₅₀ = 37.5 ± 04.7 | CC₅₀ = 36.0 ± 04.0 |
| 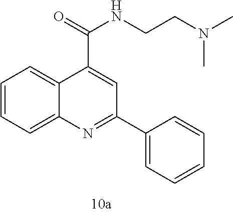 [SMILES: CN(C)CCNC(=O)c1cc(-c2ccccc2)nc2ccccc12] | EC₅₀ = 0.4 ± 0.2 CC₅₀ = 73.7 ± 19.1 SI = 184.3 | EC₅₀ = 0.3 ± 0.09 CC₅₀ = 73.7 ± 19.1 SI = 245.7 | EC₅₀ = 0.6 ± 0.1 CC₅₀ = 73.7 ± 19.1 SI = 122.8 | EC₅₀ = 0.2 ± 0.07 CC₅₀ = 73.7 ± 19.1 SI = 368.5 | EC₅₀ = 0.6 ± 0.1 CC₅₀ = 73.7 ± 19.1 SI = 122.8 | CC₅₀ = 121.1 ± 6.2 | CC₅₀ = 68.7 ± 3.8 |
| 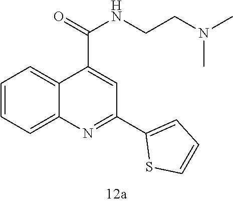 [SMILES: CN(C)CCNC(=O)c1cc(-c2cccs2)nc2ccccc12] | EC₅₀ = 0.04 ± 0.01 CC₅₀ = 42.3 ± 14.3 SI = 1057.5 | EC₅₀ = 0.06 ± 0.03 CC₅₀ = 42.3 ± 14.3 SI = 705 | EC₅₀ = 0.05 ± 0.03 CC₅₀ = 42.3 ± 14.3 SI = 846 | EC₅₀ = 0.06 ± 0.04 CC₅₀ = 42.3 ± 14.3 SI = 705 | EC₅₀ = 0.05 ± 0.03 CC₅₀ = 42.3 ± 14.3 SI = 846 | CC₅₀ = 98.8 ± 3.5 | CC₅₀ = 51.2 ± 2.0 |
| 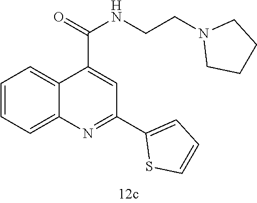 [SMILES: O=C(NCCN1CCCC1)c1cc(-c2cccs2)nc2ccccc12] | EC₅₀ = 0.06 ± 0.03 CC₅₀ = 40.0 ± 13.1 SI = 666.7 | EC₅₀ = 0.06 ± 0.02 CC₅₀ = 40.0 ± 13.1 SI = 666.7 | EC₅₀ = 0.05 ± 0.03 CC₅₀ = 40.0 ± 13.1 SI = 800 | EC₅₀ = 0.05 CC₅₀ = 40.0 ± 13.1 SI = 800 | EC₅₀ = 0.07 ± 0.03 CC₅₀ = 40.0 ± 13.1 SI = 571.4 | CC₅₀ = 83.2 ± 4.1 | CC₅₀ = 41.7 ± 4.3 |

TABLE 8
Antiviral activity of potent leads against different serotypes of EV-D68 in neuronal cells

| | Antiviral activity and cytotoxicity in neuronal cells | | | |
|---|---|---|---|---|
| | US/KY/14-18953 | US/KY/14-18953 | US/MO/14-18947 | US/MO/14-18947 |
| | in A172 cells | in SH-SY5Y cells | in A172 cells | in SH-SY5Y cells |
| Compound ID | (μM) | (μM) | (μM) | (μM) |
| 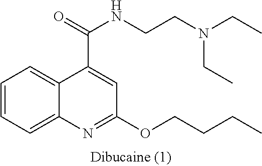 [SMILES: CCCCOc1cc(C(=O)NCCN(CC)CC)c2ccccc2n1] | EC₅₀ = 3.7 ± 1.5 CC₅₀ >30.0 SI >8.1 | EC₅₀ = 1.7 ± 0.8 CC₅₀ >30.0 SI >17.6 | EC₅₀ = 1.9 ± 0.1 CC₅₀ >30.0 SI >15.8 | EC₅₀ = 1.9 ± 0.3 CC₅₀ >30.0 SI >15.8 |
| 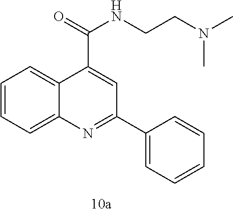 [SMILES: CN(C)CCNC(=O)c1cc(-c2ccccc2)nc2ccccc12] | EC₅₀ = 0.4 ± 0.1 CC₅₀ = 88.4 ± 31.6 SI = 221.0 | EC₅₀ = 0.4 ± 0.1 CC₅₀ >300.0 SI >750.0 | EC = 0.3 ± 0.02 CC₅₀ = 88.4 ± 31.6 SI = 294.7 | EC₅₀ = 0.2 ± 0.07 CC₅₀ >300.0 SI >1500.0 |
| 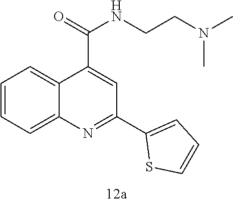 [SMILES: CN(C)CCNC(=O)c1cc(-c2cccs2)nc2ccccc12] | EC₅₀ = 0.02 ± 0.01 CC₅₀ = 73.8 ± 41.0 SI = 3690.0 | EC₅₀ = 0.1 ± 0.05 CC₅₀ = 136.3 ± 69.3 SI = 1363.0 | EC₅₀ = 0.1 ± 0.01 CC₅₀ = 73.8 ± 41.0 SI = 738.0 | EC₅₀ = 0.01 ± 0.008 CC₅₀ = 136.3 ± 69.3 SI = 13630.0 |
| 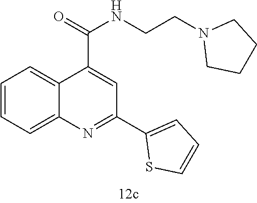 [SMILES: O=C(NCCN1CCCC1)c1cc(-c2cccs2)nc2ccccc12] | EC₅₀ = 0.05 ± 0.00 CC₅₀ = 65.4 ± 22.2 SI = 1308.0 | EC₅₀ = 0.1 ± 0.08 CC₅₀ = 93.3 ± 30.7 SI = 933.0 | EC₅₀ = 0.2 ± 0.02 CC₅₀ = 65.4 ± 22.2 SI = 327.0 | EC₅₀ = 0.02 CC₅₀ = 93.3 ± 30.7 SI = 4665.0 |

### Mechanism of Action

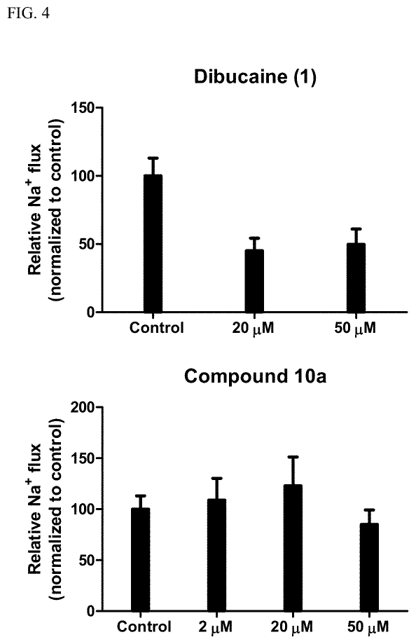

It was reported that dibucaine inhibits EV-D68 through the inhibition of genome replication by targeting the 2C protein (see, Ulferts, R.; et al., Antimicrob. Agents Chemother. 2016, 60, 2627-2638). The EV-D68 2C protein is a highly conserved viral protein that plays multiple functions in genome replication. It has been implicated in RNA replication, membrane rearrangements, encapsidation, and uncoating (see, Van der Linden, L.; et al., Viruses 2015, 7, 4529-4562). Inhibition of viral 2C protein is expected to inhibit viral RNA and protein synthesis. To test this hypothesis and explore the cellular antiviral mechanism of dibucaine analogs 10a, 12a and 12c, experiments detected viral capsid protein VP1 expression level by immunofluorescence imaging and western blot, and viral RNA levels by RT-qPCR (FIG. 3). Compared to DMSO control, 3 μM of dibucaine significantly reduced the fluorescent signal of VP1 at 9 hours post infection (hpi) (FIG. 3A). The reduction of VP1 protein level was also independently confirmed in western blot experiment (FIG. 3B), whereas 3 μM of dibucaine nearly completely inhibited VP1 expression (FIG. 3B) while 1 μM dibucaine had no effect. In contrast, when tested at 1 μM, dibucaine analogs, 10a, 12a and 12c, completely blocked the VP1 protein expression (FIG. 3A & 3B) as shown by immunofluorescence imaging and western blot. Similarly, RT-qPCR quantification of viral RNAs showed that 1 μM of dibucaine analogs, 10a, 12a and 12c, completely inhibited the viral RNA synthesis (>99%), whereas 3 μM of dibucaine reduced viral RNA level by ˜80% (FIG. 3C). Taken together, these results confirmed that dibucaine and its analogs share similar mechanism of action, and the analogs had more potent antiviral activity than dibucaine.

### Potential Side Effects

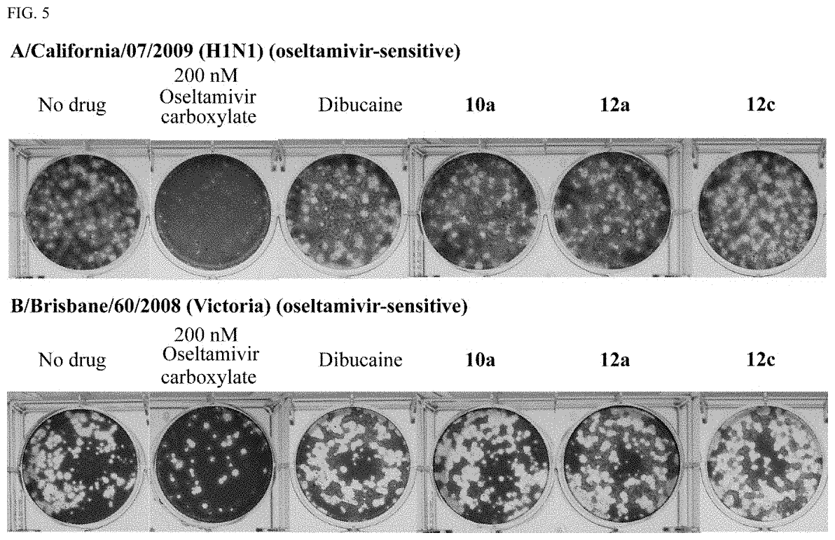

Dibucaine is an FDA-approved topical anesthetic drug. Its anesthetic activity is due to binding and inhibiting sodium channels within neuronal cell membranes. For the purpose of antiviral development, inhibition of the sodium channel might be an unwanted side effect. As the three lead compounds 10a, 12a, and 12c were derived from dibucaine, there was a concern that they might have anesthetic side effect by inhibiting the sodium channel. To ease this doubt, experiments were conducted that chose one representative compound 10a and tested its sodium channel inhibition using the standard sodium flux assay. Dibucaine was included as a positive control. In this assay, rat dorsal root ganglia (DRG) neurons were incubated with various concentrations of testing compounds. Sodium channels were then activated by the sodium channel agonist veratridine. The resulting sodium flux was measured indirectly by quantifying the sodium channel triggered calcium flux, which was measured by a calcium-specific fluorescence dye (FIG. 4). It was found that dibucaine potently inhibited sodium current conductance at both 20 μM and 50 μM, while compounds 10a had minimal to no effect from 2 μM to 50 μM, nearly 100-fold higher than its antiviral EC₅₀ value. These results confirmed that the quinoline lead compounds identified from this study such as 10a are promising candidates with no potential anesthetic side effects, suggesting they are promising for further development.

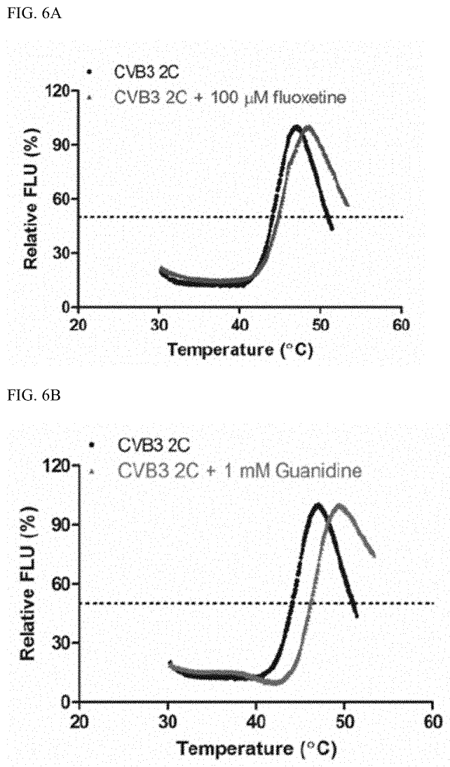

To address the concern that the optimized lead compounds might be promiscuous antivirals, experiments were conducted that tested the three lead compounds 10a, 12a, and 12c against two influenza viruses, the influenza A virus A/California/07/2009 (H1N1), and the influenza B virus B/Brisbane/60/2008 (Victoria), in plaque assay (see, Wang, Y.; et al., J. Med. Chem. 2018, 61, 1074-1085). It was found that dibucaine and the three lead compounds had no inhibition against these two strains of influenza viruses when tested at 10 μM (FIG. 5), indicating the antiviral activity of the lead compounds 10a, 12a, and 12c, against EV-D68 was not due to non-specific effect of these compounds.

### Conclusions

The magnitude and severity of EV-D68 outbreak in recent years underscores a need for effective antivirals, not only to be used for prophylaxis, but also for the treatment of EV-D68 infection. In this study, experiments were conducted that chose a previously reported hit compound, dibucaine, as a starting point for the structure-activity relationship studies. Dibucaine was identified as an EV-D68 antiviral from a drug repurposing phenotypic screening. However, the moderate antiviral activity and a low selectivity index of dibucaine impeded its clinical use as an antiviral. As such, we aim to optimize the potency and selectivity index of dibucaine through structure-activity relationship studies. This effort led to the identification of three lead compounds 10a, 12a, and 12c, with significantly improved antiviral efficacy and selectivity index (EC_{50<1} μM, SI>180). The antiviral mechanism of action of these three compounds were investigated by immunofluorescence staining, western blot, and RT-qPCR assays. Collectively, all three compounds inhibited viral protein and RNA synthesis, consistent with the proposed mechanism of 2C protein inhibition. Nevertheless, to confirm the mechanism of action, serial viral passage experiments need to be designed to select resistant mutant in viral 2C protein. Alternatively, direct binding needs to be measured between lead compounds and viral 2C protein. The lead compounds might either affect the function of viral 2C protein directly or disrupt essential interactions between viral 2C protein and host factors. Such experiments are ongoing and will be reported when available. These three compounds represent the most potent and select EV-D68 2C inhibitors reported so far. Of particular highlights are the easy synthesis accessibility of these compounds which can be made in two steps from commercial available materials, broad range antiviral activity against all five contemporary EV-D68 strains, and consistent antiviral activity across different cell lines including muscle cells (RD) and neuronal cells (A172 and SH-SYSY). It has been shown that recent circulating EV-D68 strains such as the ones tested in this study acquired the fitness of replication in neuronal cells. Therefore it is important for an antiviral drug candidate to show consistent antiviral activity in different cell lines. It should be pointed out that although EV-D68 infection has a direct link with AFM (see, Dyda, A.; et al., Eurosurveillance 2018, 23, 16-24), it is not clear whether AFM is a result of viral replication in the CNS (spinal cord and brain) or due to inflammation caused by viral infection. Therefore it is desired to develop both blood-brain-barrier (BBB) permeable and impermeable EV-D68 antivirals and test them in mouse model studies to dissect whether BBB permeability is essential for AFM treatment. The compounds reported herein represent not only lead candidates for further translational development into EV-D68 antivirals, but also valuable chemical probes to help understand the structure, function, and mechanism of EV-D68 2C protein. Accordingly, future efforts will focus on optimizing the in vitro and in vivo pharmacokinetic properties of this series of compounds and evaluating their in vivo antiviral efficacy in mouse model studies. On another hand, the structure of the EV-D68 2C protein is currently unknown. The inhibitors discovered herein might be able to stabilize the 2C protein and help facilitate its structure determination by either NMR or X-ray crystallography. A high-resolution crystal structure of EV-D68 2C is highly desired as it will be invaluable in guiding the rational design of next-generation of 2C inhibitors.

### Experimental Section

Chemistry.

Chemicals were ordered from commercial sources and were used without further purification. Synthesis procedures for reactions described in Scheme 1 were shown below. All final compounds were purified by flash column chromatography. ¹H and ¹³C NMR spectra were recorded on a Bruker-400 NMR spectrometer. Chemical shifts are reported in parts per million referenced with respect to residual solvent (CD₃OD) 3.31 ppm, (DMSO-d₆) 2.50 ppm, and (CDCl₃) 7.24 ppm or from internal standard tetramethylsilane (TMS) 0.00 ppm. The following abbreviations were used in reporting spectra: s, singlet; d, doublet; t, triplet; q, quartet; m, multiplet; dd, doublet of doublets; ddd, doublet of doublet of doublets. All reactions were carried out under N₂ atmosphere unless otherwise stated. HPLC-grade solvents were used for all reactions. Flash column chromatography was performed using silica gel (230-400 mesh, Merck). High-resolution mass spectra were obtained using the positive ESI method for all the compounds, obtained in an Ion Cyclotron Resonance (ICR) spectrometer. Low-resolution mass spectra were obtained using an ESI technique on a 3200 Q Trap LC/MS/MS system (Applied Biosystems). The purity was assessed by using a Shimadzu LC-MS with a Waters XTerra MS C-18 column (part no. 186000538), 50 mm×2.1 mm, at a flow rate of 0.3 mL/min;)\, =250 and 220 nm; mobile phase A, 0.1% formic acid in H₂O, and mobile phase B′, 0.1% formic in 60% 2-propanol, 30% CH₃CN, and 9.9% H₂O. All compounds submitted for antiviral CPE assay, cytotoxicity assay, and mechanistic studies were confirmed to be >95.0% purity by LC-MS traces.

### Synthesis Procedures.

General Procedure of Amide Coupling.

To a DMF solution of carboxylic acid (1 mmol) was added HATU (1 mmol) and DIEA (1.2 mmol). After stirring for 2 minutes, amine (1 mmol) was added. The resulting solution was stirred overnight at room temperature. The reaction was diluted with dichloromethane and extracted with aqueous NaHCO₃ solution, followed by brine. The organic layer was dried over MgSO4, filtered, and concentrated under reduced pressure. The crude product was purified by flash column chromatography (1-10% CH₃OH/CH₂Cl₂) to give the final product.

General Procedure of Suzuki-Miyaura Cross Coupling.

To a solution of 2-chloroquinoline (1 mmol) and boronic acid (Immo′) in 1,4-dioxane in a microwave reaction vial was added an aqueous solution of K₂CO₃ (2 mmol). The resulting solution was purged with N₂ for 5 minutes. The catalyst, Pd(PPh₃)₄ (0.1 mmol), was added in one portion. The vial was capped and heated to 140° C. for 30 minutes with microwave irradiation. After cooling down to room temperature, the reaction solution was diluted with CH₂Cl₂ and extracted with aqueous NaHCO₃ solution, followed by brine. The organic layer was dried over MgSO4, filtered, and concentrated under reduced pressure. The crude product was purified by flash column chromatography (1-10% CH₃OH/CH₂Cl₂) to give the final product.

Compounds 4, 8, 10, 12, and 15 were synthesized using the general procedure of amide coupling.

Compounds 6 and 19 were synthesized using the general procedure of Suzuki-Miyaura cross coupling.

Procedure for the Synthesis of Compound 5 Through Method 1.

Sodium metal (10 mmol) was cut into small pieces and was added to the alcohol solution. The solution was stirred at room temperature under N₂ until all the sodium metal was dissolved (heating to 60° C. when necessary). 2-chloro-N-[2-(diethylamino)ethyl]quinoline-4-carboxamide (4) (1 mmol) was added in one portion and the resulting solution was heated to reflux overnight. Reaction was quenched with H₂O and excess of alcohol was removed under reduced pressure. The crude product was diluted with CH₂Cl₂ and extracted with aqueous NaHCO₃ solution, followed by brine. The organic layer was dried over MgSO4, filtered, and concentrated under reduced pressure. The crude product was purified by flash column chromatography (1-10% CH₃OH/CH₂Cl₂) to give the final product.

Procedure for the Synthesis of Compound 5 Through Method 2.

To a solution of alcohol (2 mmol) in DMF was added NaH (3 mmol), and the mixture was stirred at room temperature for 1 hour. A solution of 2-chloro-N-[2-(diethylamino)ethyl]quinoline-4-carboxamide (4) (1 mmol) in DMF was added dropwise, and the resulting solution was stirred at 80° C. for 4 hours. The reaction mixture was quenched with aqueous NaHCO₃ solution, and extracted with CH₂Cl₂. The organic layer was dried over MgSO4, filtered, and concentrated under reduced pressure. The crude product was purified by flash column chromatography (1-10% CH₃OH/CH₂Cl₂) to give the final product.

Procedure for the Synthesis of Compound 14.

To a solution of isatin (1 mmol) and acetophenone (1 mmol) in ethanol was added the aqueous solution of KOH (10 mmol). The reaction mixture was heated to 125° C. under microwave irradiation for 20 minutes. After cooling down to room temperature, the reaction was quenched with HCl. The precipitate was collected by filtration, washed sequentially with water, ethyl acetate, and dichloromethane. The crude product was purified by flash column chromatography (10-20% CH₃OH/CH₂Cl₂) to give the final product.

Procedure for the Synthesis of Compound 17.

A solution of 5-fluoroisatin (1 mmol) and malonic acid (3 mmol) in acetic acid was refluxed for 16 hours. Acetic acid was removed under reduced pressure, and the residue was diluted with water. Insoluble solid was collected by filtration and was suspended in saturated NaHCO₃ aqueous solution. The mixture was filtered and the filtrate was acidified to pH 1 using concentrated HCl (12 N). Precipitate formed was filtered and washed with water. The resulting solid was further purified by flash column chromatography (10-20% CH₃OH/CH₂Cl₂) to give the final product.

General Procedure for the Synthesis of Compound 18.

To a solution of 6-fluoro-2-hydroxyquinoline-4-carboxylic acid (17) (1 mmol) in CH₂Cl₂ was added a few drops of DMF and thionyl chloride (4 mmol). The mixture was refluxed for 3 hours. Solvent was removed under reduced pressure and the resulting acid chloride was dissolved in THF. Amine (3 mmol) was added and the reaction mixture was stirred at room temperature overnight. Solvent was removed under reduced pressure and the resulting residue was dissolved in CH₂Cl₂. The solution was extracted with aqueous NaHCO₃ solution, followed by brine. The organic layer was dried over MgSO4, filtered, and concentrated under reduced pressure. The crude product was purified by flash column chromatography (1-10% CH₃OH/CH₂Cl₂) to give the final product.

### 2-chloro-N-[2-(diethylamino)ethyl]quinoline-4-carboxamide (4)

Yield: 82%. ¹H NMR (400 MHz, CD₃OD-d₄) δ 8.18 (d, J=8.9 Hz, 1H), 7.94 (d, J=8.4 Hz, 1H), 7.79 (t, J=7.7 Hz, 1H), 7.63 (t, J=8.3 Hz, 1H), 7.58 (s, 1H), 3.62 (t, J=6.9 Hz, 2H), 2.89 (t, J=6.9 Hz, 2H), 2.78 (q, J=7.2 Hz, 4H), 1.14 (t, J=7.2 Hz, 6H). ¹³C NMR (101 MHz, CD₃OD) δ 168.19, 151.20, 149.28, 146.61, 132.35, 129.29, 128.99, 126.69, 124.63, 121.29, 52.30, 48.20, 37.90, 11.18. C₁₆H₂₀ClN₃O, EI-MS: m/z (M+H⁺): 306.8 (calculated), 306.8 (found).

### N-[2-(diethylamino)ethyl]-2-methoxyquinoline-4-carboxamide (5a)

Yield: 72%. ¹H NMR (400 MHz, CD₃OD-d₄) δ 8.06 (dd, J=8.4, 1.4 Hz, 1H), 7.85 (dd, J=8.5, 1.5 Hz, 1H), 7.66 (ddd, J=8.4, 6.9, 1.5 Hz, 1H), 7.43 (ddd, J=8.3, 6.9, 1.3 Hz, 1H), 7.02 (s, 1H), 4.07 (s, 3H), 3.66-3.56 (m, 2H), 2.90 (t, J=7.0 Hz, 2H), 2.81 (q, J=7.2 Hz, 4H), 1.16 (t, J=7.2 Hz, 6H). ¹³C NMR (101 MHz, CD₃OD) δ 169.71, 163.13, 148.38, 146.29, 131.18, 128.50, 126.32, 125.79, 122.73, 112.16, 54.12, 52.35, 48.32, 37.75, 11.13. C₁₇H₂₃N₃O₂, HRMS (ESI): m/z (M+H⁺): 302.1869 (calculated), 302.1863 (found).

### N-[2-(diethylamino)ethyl]-2-ethoxyquinoline-4-carboxamide (5b)

Yield: 84%. ¹H NMR (400 MHz, DMSO-d₆) δ 8.72-8.61 (m, 1H), 8.08 (dd, J=8.3, 1.4 Hz, 1H), 7.87-7.74 (m, 1H), 7.68 (ddd, J=8.3, 6.8, 1.5 Hz, 1H), 7.44 (ddd, J=8.3, 6.8, 1.3 Hz, 1H), 6.99 (d, J=11.4 Hz, 1H), 4.49 (q, J=7.0 Hz, 2H), 3.46-3.32 (m, 2H), 2.75-2.52 (m, 6H), 1.39 (t, J=7.0 Hz, 3H), 1.01 (t, J=7.1 Hz, 6H). ¹³C NMR (101 MHz, DMSO) δ 166.03, 160.91, 146.40, 145.71, 130.05, 130.00, 127.08, 125.61, 125.58, 124.51, 124.41, 121.43, 110.87, 61.50, 53.37, 51.29, 46.57, 14.39, 11.62. C₁₈H₂₅N₃O₂, HRMS (ESI): m/z (M+H⁺): 316.2025 (calculated), 316.2020 (found).

### N-[2-(diethylamino)ethyl]-2-(2,2,2-trifluoroethoxy)quinoline-4-carboxamide (5c)

Yield: 71%. ¹H NMR (400 MHz, DMSO-d₆) δ 8.81-8.67 (m, 1H), 8.14 (ddd, J=8.3, 1.5, 0.6 Hz, 1H), 7.86 (ddd, J=8.5, 1.4, 0.7 Hz, 1H), 7.75 (ddd, J=8.4, 6.9, 1.5 Hz, 1H), 7.52 (ddd, J=8.3, 6.9, 1.3 Hz, 1H), 7.17 (s, 1H), 5.20 (q, J=9.1 Hz, 2H), 3.53-3.33 (m, 2H), 2.76-2.52 (m, 6H), 1.01 (t, J=7.1 Hz, 6H). ¹³C NMR (101 MHz, DMSO): δ 165.68, 158.94, 146.77, 145.60, 130.55, 127.19, 125.76, 125.41, 125.37, 122.65, 122.10, 110.08, 61.91, 61.56, 61.22, 60.87, 51.30, 46.57, 37.17, 11.62. C₁₈H₂₂F₃N₃O₂, HRMS (ESI): m/z (M+H⁺): 370.1742 (calculated), 370.1737 (found).

### N-[2-(diethylamino)ethyl]-2-propoxyquinoline-4-carboxamide (5d)

Yield: 87%. ¹H NMR (400 MHz, DMSO-d₆) δ 8.80-8.64 (m, 1H), 8.08 (dd, J=8.3, 1.4 Hz, 1H), 7.79 (dd, J=8.4, 1.2 Hz, 1H), 7.68 (ddd, J=8.4, 6.9, 1.5 Hz, 1H), 7.44 (ddd, J=8.2, 6.8, 1.3 Hz, 1H), 7.02 (s, 1H), 4.39 (t, J=6.6 Hz, 2H), 3.51-3.35 (m, 2H), 2.90-2.52 (m, 6H), 1.79 (p, J=7.1 Hz, 2H), 1.14-0.91 (m, 9H). ¹³C NMR (101 MHz, DMSO) δ 166.08, 161.08, 146.40, 145.54, 130.00, 127.08, 125.57, 124.42, 121.42, 110.94, 67.22, 51.08, 48.57, 46.56, 21.75, 11.30, 10.43. C₁₉H₂₇N₃O₂, HRMS (ESI): m/z (M+H⁺): 330.2182 (calculated), 330.2176 (found).

### N-[2-(diethylamino)ethyl]-2-(propan-2-yloxy)quinoline-4-carboxamide (5e)

Yield: 80%. ¹H NMR (400 MHz, CD₃OD-d₄) δ 8.04 (dd, J=8.4, 1.4 Hz, 1H), 7.81 (dd, J=8.6, 1.2 Hz, 1H), 7.65 (ddd, J=8.4, 6.9, 1.4 Hz, 1H), 7.41 (ddd, J=8.3, 6.9, 1.3 Hz, 1H), 6.96 (s, 1H), 5.55 (p, J=6.2 Hz, 1H), 3.59 (dd, J=7.6, 6.4 Hz, 2H), 2.86 (dd, J=7.7, 6.3 Hz, 2H), 2.77 (q, J=7.2 Hz, 4H), 1.41 (d, J=6.2 Hz, 6H), 1.15 (t, J=7.2 Hz, 6H). ¹³C NMR (101 MHz, MeOD) δ 169.79, 162.31, 148.50, 146.28, 131.06, 128.49, 126.24, 125.57, 122.54, 112.77, 69.59, 52.39, 48.26, 37.88, 22.30, 11.28. C₁₉H₂₇N₃O₂, HRMS (ESI): m/z (M+H⁺): 330.2182 (calculated), 330.2176 (found).

### 2-(butan-2-yloxy)-N-[2-(diethylamino)ethyl]quinoline-4-carboxamide Yield

72%. ¹H NMR (400 MHz, DMSO-d₆) δ 8.85-8.67 (m, 1H), 8.06 (dd, J=8.3, 1.4 Hz, 1H), 7.77 (dd, J=8.5, 1.2 Hz, 1H), 7.67 (ddd, J=8.4, 6.8, 1.5 Hz, 1H), 7.43 (ddd, J=8.3, 6.9, 1.3 Hz, 1H), 6.99 (s, 1H), 5.35 (q, J=6.2 Hz, 1H), 3.57-3.36 (m, 2H), 2.98-2.52 (m, 6H), 1.81-1.61 (m, 2H), 1.33 (d, J=6.2 Hz, 3H), 1.06 (d, J=7.5 Hz, 6H), 0.95 (t, J=7.4 Hz, 3H). ¹³C NMR (101 MHz, DMSO) δ 166.16, 160.74, 146.42, 129.98, 127.06, 125.50, 124.33, 121.26, 111.41, 72.37, 46.54, 28.38, 19.17, 9.62. C₂₀H₂₉N₃O₂, HRMS (ESI): m/z (M+H⁺): 344.2338 (calculated), 344.2333 (found).

### 2-(cyclopropylmethoxy)-N-[2-(diethylamino)ethyl]quinoline-4-carboxamide (5g)

Yield: 71%. ¹H NMR (400 MHz, DMSO-d₆) δ 8.79-8.64 (m, 1H), 8.08 (dd, J=8.3, 1.4 Hz, 1H), 7.78 (dd, J=8.4, 1.2 Hz, 1H), 7.67 (ddd, J=8.4, 6.8, 1.5 Hz, 1H), 7.44 (ddd, J=8.3, 6.8, 1.3 Hz, 1H), 7.03 (s, 1H), 4.28 (d, J=7.2 Hz, 2H), 3.53-3.37 (m, 2H), 2.88-2.57 (m, 6H), 1.38-1.25 (m, 1H), 1.04 (t, J=7.1 Hz, 6H), 0.66-0.51 (m, 2H), 0.45-0.32 (m, 2H). ¹³C NMR (101 MHz, DMSO) δ 166.10, 161.00, 146.36, 130.02, 127.05, 125.56, 124.43, 121.40, 110.99, 70.30, 54.90, 51.03, 46.57, 11.17, 9.96, 3.19. C₂₀H₂₇N₃O₂, HRMS (ESI): m/z (M+H⁺): 342.2182 (calculated), 342.2176 (found).

### N-[2-(diethylamino)ethyl]-2-(pentan-3-yloxy)quinoline-4-carboxamide (5h)

Yield: 77%. ¹H NMR (400 MHz, DMSO-d₆) δ 8.74-8.62 (m, 1H), 8.06 (dd, J=8.3, 1.4 Hz, 1H), 7.75 (dd, J=8.5, 1.2 Hz, 1H), 7.66 (ddd, J=8.4, 6.8, 1.5 Hz, 1H), 7.42 (ddd, J=8.2, 6.8, 1.3 Hz, 1H), 6.97 (s, 1H), 5.30 (p, J=6.0 Hz, 1H), 3.47-3.32 (m, 2H), 2.72-2.51 (m, 6H), 1.82-1.61 (m, 4H), 1.01 (t, J=7.1 Hz, 6H), 0.92 (t, J=7.4 Hz, 6H). ¹³C NMR (101 MHz, DMSO) δ 166.08, 162.24, 161.18, 146.36, 145.74, 129.93, 127.03, 125.51, 124.25, 121.33, 111.19, 76.67, 51.23, 46.57, 37.00, 35.74, 30.73, 25.67, 11.52, 9.46. C₂₁H₃₁N₃O₂, HRMS (ESI): m/z (M+H⁺): 358.2495 (calculated), 358.2489 (found).

### 2-(cyclopentyloxy)-N-[2-(diethylamino)ethyl]quinoline-4-carboxamide (5i)

Yield: 82%. ¹H NMR (400 MHz, DMSO-d₆) δ 8.75-8.60 (m, 1H), 8.08 (d, J=8.2 Hz, 1H), 7.78 (d, J=8.4 Hz, 1H), 7.67 (t, J=7.7 Hz, 1H), 7.42 (t, J=7.9 Hz, 1H), 6.94 (s, 1H), 5.65-5.54 (m, 1H), 3.49-3.31 (m, 2H), 2.80-2.53 (m, 6H), 2.09-1.95 (m, 2H), 1.83-1.68 (m, 4H), 1.68-1.54 (m, 2H), 1.02 (t, J=7.2 Hz, 6H). ¹³C NMR (101 MHz, DMSO) δ 166.07, 160.68, 146.48, 145.48, 129.93, 127.16, 125.54, 124.32, 121.29, 111.30, 77.53, 54.89, 51.17, 46.57, 36.90, 32.36, 23.48, 11.43. C₂₁H₂₉N₃O₂, HRMS (ESI): m/z (M+H⁺): 356.2338 (calculated), 356.2333 (found).

### N-[2-(diethylamino)ethyl]-2-(2-ethoxyethoxy)quinoline-4-carboxamide (5j)

Yield: 62%. ¹H NMR (400 MHz, DMSO-d₆) δ 8.78-8.64 (m, 1H), 8.09 (ddd, J=7.4, 5.8, 1.4 Hz, 1H), 7.79 (dd, J=8.4, 1.4 Hz, 1H), 7.68 (dddd, J=8.4, 6.9, 3.2, 1.5 Hz, 1H), 7.44 (dddd, J=8.3, 6.9, 4.0, 1.3 Hz, 1H), 7.02 (d, J=18.9 Hz, 1H), 4.63-4.41 (m, 2H), 3.83-3.70 (m, 2H), 3.51 (q, J=7.0 Hz, 2H), 3.47-3.31 (m, 2H), 2.83-2.54 (m, 6H), 1.13 (t, J=7.0 Hz, 3H), 1.03 (t, J=7.1 Hz, 6H). ¹³C NMR (101 MHz, DMSO) δ 166.02, 160.86, 146.27, 145.66, 130.06, 127.09, 125.60, 124.53, 121.50, 110.92, 68.03, 65.61, 65.13, 61.51, 51.13, 46.57, 15.09, 14.39. C₂₀H₂₉N₃O₃, HRMS (ESI): m/z (M+H⁺): 360.2287 (calculated), 360.2282 (found).

### 2-(benzyloxy)-N-[2-(diethylamino)ethyl]quinoline-4-carboxamide (5k)

Yield: 84%. ¹H NMR (400 MHz, CD₃OD-d₄) δ 8.07 (dd, J=8.4, 1.4 Hz, 1H), 7.86 (dd, J=8.5, 1.3 Hz, 1H), 7.66 (ddd, J=8.4, 6.9, 1.4 Hz, 1H), 7.56-7.47 (m, 2H), 7.47-7.41 (m, 1H), 7.41-7.33 (m, 2H), 7.33-7.24 (m, 1H), 7.08 (s, 1H), 5.54 (s, 2H), 3.60 (t, J=7.0 Hz, 2H), 2.90 (t, J=7.0 Hz, 2H), 2.80 (q, J=7.2 Hz, 4H), 1.15 (t, J=7.2 Hz, 6H). ¹³C NMR (101 MHz, MeOD) δ 169.71, 162.46, 148.30, 146.41, 138.44, 131.23, 129.43, 129.25, 128.95, 128.58, 126.31, 125.89, 122.84, 112.31, 68.92, 52.32, 48.36, 37.71, 11.07. C₂₃H₂₇N₃O₂, HRMS (ESI): m/z (M+H⁺): 378.2182 (calculated), 378.2176 (found).

### N-[2-(diethylamino)ethyl]-2-(1H-pyrazol-5-yl)quinoline-4-carboxamide (6a)

Yield: 71%. ¹H NMR (400 MHz, CD₃OD-d₄) δ 8.25-8.02 (m, 3H), 7.84-7.72 (m, 2H), 7.59 (ddd, J=8.3, 6.9, 1.2 Hz, 1H), 7.12 (d, J=2.3 Hz, 1H), 3.60 (dd, J=8.0, 6.3 Hz, 2H), 2.79 (dd, J=7.9, 6.4 Hz, 2H), 2.68 (q, J=7.2 Hz, 4H), 1.12 (t, J=7.2 Hz, 6H). ¹³C NMR (101 MHz, MeOD) δ 169.88, 149.49, 144.51, 131.44, 130.09, 128.27, 126.43, 125.09, 117.66, 105.41, 52.51, 48.14, 38.33, 11.68. C₁₉H₂₃N₅O, HRMS (ESI): m/z (M+H⁺): 338.1981 (calculated), 338.1975 (found).

### N-[2-(diethylamino)ethyl]-2-(1-methyl-1H-pyrazol-4-yl)quinoline-4-carboxamide (6b)

Yield: 78%. ¹H NMR (400 MHz, DMSO-d₆) δ 8.81-8.71 (m, 1H), 8.52 (s, 1H), 8.20 (s, 1H), 8.14 (ddd, J=8.4, 1.5, 0.6 Hz, 1H), 7.97 (ddd, J=8.5, 1.3, 0.6 Hz, 1H), 7.86 (s, 1H), 7.74 (ddd, J=8.4, 6.8, 1.4 Hz, 1H), 7.53 (ddd, J=8.2, 6.8, 1.3 Hz, 1H), 3.94 (s, 3H), 3.52-3.42 (m, 2H), 2.82-2.70 (m, 2H), 2.70-2.53 (m, 4H), 1.04 (t, J=7.1 Hz, 6H). ¹³C NMR (101 MHz, DMSO) δ 166.66, 151.51, 147.97, 143.17, 137.82, 130.62, 129.93, 128.64, 125.95, 125.50, 122.83, 122.68, 116.48, 51.29, 46.60, 38.83, 36.97, 11.45. C₂₀H₂₅N₅O, HRMS (ESI): m/z (M+H⁺): 352.2137 (calculated), 352.2132 (found).

### N-[2-(diethylamino)ethyl]-2-(1-methyl-1H-pyrazol-5-yl)quinoline-4-carboxamide (6c)

Yield: 65%. ¹H NMR (400 MHz, DMSO-d₆) δ 8.87-8.79 (m, 1H), 8.24 (ddd, J=8.4, 1.5, 0.6 Hz, 1H), 8.11 (ddd, J=8.4, 1.3, 0.6 Hz, 1H), 7.97 (s, 1H), 7.83 (ddd, J=8.4, 6.9, 1.5 Hz, 1H), 7.66 (ddd, J=8.3, 6.9, 1.3 Hz, 1H), 7.58 (d, J=2.0 Hz, 1H), 7.10 (d, J=2.0 Hz, 1H), 4.35 (s, 3H), 3.53-3.43 (m, 2H), 2.80-2.70 (m, 2H), 2.70-2.54 (m, 4H), 1.04 (t, J=7.1 Hz, 6H). ¹³C NMR (101 MHz, DMSO) δ 166.24, 148.85, 147.21, 143.44, 139.95, 137.91, 130.39, 129.31, 127.42, 125.53, 122.95, 118.36, 108.21, 51.23, 46.60, 39.91, 37.00, 11.37. C₂₀H₂₅N₅O, HRMS (ESI): m/z (M+H⁺): 352.2137 (calculated), 352.2132 (found).

### N-[2-(diethylamino)ethyl]-2-(thiophen-2-yl)quinoline-4-carboxamide (6d)

Yield: 82%. ¹H NMR (400 MHz, DMSO-d₆) δ 9.00-8.88 (br s, 1H), 8.26-8.15 (m, 2H), 8.09 (d, J=3.7 Hz, 1H), 8.06-7.94 (m, 1H), 7.87-7.72 (m, 2H), 7.62-7.55 (m, 1H), 7.27-7.21 (m, 1H), 3.70-3.46 (m, 2H), 3.04-2.57 (m, 6H), 1.11 (t, J=7.1 Hz, 6H). ¹³C NMR (101 MHz, DMSO) δ 166.51, 151.61, 147.64, 144.25, 143.00, 130.33, 130.02, 128.77, 128.59, 127.65, 126.72, 125.56, 123.31, 115.64, 50.81, 46.55, 36.25, 10.60. C₂₀H₂₃N₃OS, HRMS (ESI): m/z (M+H⁺): 354.1640 (calculated), 354.1629 (found).

### 2-(5-chlorothiophen-2-yl)-N-[2-(diethylamino)ethyl]quinoline-4-carboxamide (6e)

Yield: 85%. ¹HNMR (400 MHz, CD₃OD-d₄) δ 8.15 (ddd, J=8.4, 1.4, 0.7 Hz, 1H), 8.09-7.95 (m, 2H), 7.75 (ddd, J=8.4, 6.9, 1.5 Hz, 1H), 7.70 (d, J=4.0 Hz, 1H), 7.56 (ddd, J=8.3, 6.9, 1.3 Hz, 1H), 7.05 (d, J=4.0 Hz, 1H), 3.70 (dd, J=7.4, 6.4 Hz, 2H), 3.04 (t, J=6.9 Hz, 2H), 2.93 (q, J=7.2 Hz, 4H), 1.22 (t, J=7.2 Hz, 6H). ¹³C NMR (101 MHz, CD₃OD) δ 169.95, 152.46, 149.54, 144.76, 143.89, 134.84, 131.61, 130.19, 128.90, 128.25, 127.47, 126.36, 124.77, 115.92, 52.32, 48.43, 37.57, 10.76. C₂₀H₂₂ClN₃OS, HRMS (ESI): m/z (M+H⁺): 388.1250 (calculated), 389.1245 (found).

### N-[2-(diethylamino)ethyl]-2-(thiophen-3-yl)quinoline-4-carboxamide (0

Yield: 86%. ¹H NMR (400 MHz, CD₃OD-d₄) δ 8.21 (dd, J=3.0, 1.3 Hz, 1H), 8.14 (dd, J=8.4, 1.4 Hz, 1H), 8.09-8.01 (m, 1H), 7.98 (s, 1H), 7.88 (dd, J=5.1, 1.3 Hz, 1H), 7.71 (ddd, J=8.5, 6.9, 1.5 Hz, 1H), 7.58-7.45 (m, 2H), 3.60 (t, J=7.0 Hz, 2H), 2.87 (t, J=7.0 Hz, 2H), 2.74 (q, J=7.2 Hz, 4H), 1.12 (t, J=7.2 Hz, 6H). ¹³C NMR (101 MHz, CD₃OD) δ 169.90, 154.33, 149.58, 144.07, 142.93, 131.35, 130.15, 128.03, 127.74, 127.72, 126.84, 126.30, 124.57, 118.37, 52.36, 48.22, 37.91, 11.19. C₂₀H₂₃N₃OS, HRMS (ESI): m/z (M+H⁺): 354.1640 (calculated), 354.1629 (found).

### N-[2-(diethylamino)ethyl]-2-(furan-3-yl)quinoline-4-carboxamide (6g)

Yield: 84%. ¹H NMR (400 MHz, DMSO-d₆) δ 8.81-8.73 (m, 1H), 8.62 (dd, J=1.6, 0.8 Hz, 1H), 8.17 (ddd, J=8.4, 1.5, 0.6 Hz, 1H), 8.03 (ddd, J=8.4, 1.3, 0.6 Hz, 1H), 7.94 (s, 1H), 7.88-7.82 (m, 1H), 7.77 (ddd, J=8.4, 6.9, 1.4 Hz, 1H), 7.58 (ddd, J=8.2, 6.9, 1.3 Hz, 1H), 7.23 (dd, J=1.9, 0.8 Hz, 1H), 3.58-3.37 (m, 2H), 2.79-2.69 (m, 2H), 2.69-2.55 (m, 4H), 1.04 (t, J=7.1 Hz, 6H). ¹³C NMR (101 MHz, DMSO) δ 167.03, 151.43, 148.29, 145.25, 143.98, 143.83, 130.52, 129.42, 127.49, 126.98, 125.99, 123.66, 117.30, 109.35, 51.80, 47.07, 37.56, 11.95. C₂₀H₂₃N₃O₂, HRMS (ESI): m/z (M+H⁺): 338.1869 (calculated), 338.1864 (found).

### N-[2-(diethylamino)ethyl]-2-(3,5-dimethyl-1,2-oxazol-4-yl)quinoline-4-carboxamide (6h)

Yield: 68%. ¹H NMR (400 MHz, DMSO-d₆) δ 8.81-8.75 (m, 1H), 8.23 (ddd, J=8.4, 1.5, 0.6 Hz, 1H), 8.08 (ddd, J=8.5, 1.3, 0.6 Hz, 1H), 7.84 (ddd, J=8.4, 6.8, 1.4 Hz, 1H), 7.76-7.54 (m, 2H), 3.59-3.39 (m, 2H), 2.76-2.68 (m, 2H), 2.71 (s, 3H), 2.68-2.56 (m, 4H), 2.52 (s, 3H), 1.04 (t, J=7.1 Hz, 6H). ¹³C NMR (101 MHz, DMSO) δ 168.81, 166.82, 159.08, 150.56, 148.27, 143.91, 130.74, 129.59, 127.62, 125.96, 123.20, 118.84, 115.81, 51.80, 47.03, 37.63, 12.89, 11.90. C₂₁H₂₆N₄O₂, HRMS (ESI): m/z (M+H⁺): 367.2134 (calculated), 367.2122 (found).

### N-[2-(diethylamino)ethyl]-2-(pyridin-4-yl)quinoline-4-carboxamide (6i)

Yield: 85%. ¹H NMR (400 MHz, DMSO-d₆) δ 8.92-8.82 (m, 1H), 8.83-8.71 (m, 2H), 8.38-8.22 (m, 4H), 8.18 (ddd, J=8.5, 1.3, 0.7 Hz, 1H), 7.86 (ddd, J=8.4, 6.9, 1.4 Hz, 1H), 7.70 (ddd, J=8.3, 6.9, 1.3 Hz, 1H), 3.55-3.43 (m, 2H), 2.81-2.70 (m, 2H), 2.68-2.54 (m, 4H), 1.04 (t, J=7.1 Hz, 6H). ¹³C NMR (101 MHz, DMSO) δ 166.36, 153.40, 150.49, 147.86, 145.07, 143.76, 130.50, 129.72, 127.89, 125.61, 124.08, 121.28, 116.65, 51.30, 46.60, 37.18, 11.51. C₂₁H₂₄N₄O, HRMS (ESI): m/z (M+H⁺): 349.2028 (calculated), 349.2022 (found).

### N-[2-(diethylamino)ethyl]-2-phenylquinoline-4-carboxamide (6ⱼ)

Yield: 92%. ¹H NMR (400 MHz, DMSO-d₆) δ 8.88-8.77 (m, 1H), 8.34-8.22 (m, 3H), 8.20-8.05 (m, 2H), 7.82 (ddd, J=8.4, 6.8, 1.4 Hz, 1H), 7.70-7.45 (m, 4H), 3.48 (q, J=6.5 Hz, 2H), 2.82-2.69 (m, 2H), 2.69-2.54 (m, 4H), 1.04 (t, J=7.1 Hz, 6H). ¹³C NMR (101 MHz, DMSO) δ 166.61, 155.74, 147.93, 143.29, 138.24, 130.13, 129.84, 129.47, 128.88, 127.23, 126.98, 125.52, 123.40, 116.58, 51.32, 46.63, 38.21, 11.55. C₂₂H₂₅N₃O, HRMS (ESI): m/z (M+H⁺): 348.2076 (calculated), 348.2069 (found).

### N-[2-(diethylamino)ethyl]-2-(2-methoxyphenyl)quinoline-4-carboxamide (6k)

Yield: 88%. ¹H NMR (400 MHz, DMSO-d₆) δ 8.69 (t, J=5.7 Hz, 1H), 8.23 (ddd, J=8.4, 1.5, 0.6 Hz, 1H), 8.10 (ddd, J=8.5, 1.3, 0.6 Hz, 1H), 7.92 (s, 1H), 7.86-7.74 (m, 2H), 7.63 (ddd, J=8.3, 6.9, 1.3 Hz, 1H), 7.49 (ddd, J=8.3, 7.3, 1.8 Hz, 1H), 7.21 (dd, J=8.4, 1.0 Hz, 1H), 7.13 (td, J=7.4, 1.0 Hz, 1H), 3.86 (s, 3H), 3.45 (q, J=6.4 Hz, 2H), 2.75-2.67 (m, 2H), 2.67-2.54 (m, 4H), 1.03 (t, J=7.1 Hz, 6H). ¹³C NMR (101 MHz, DMSO) δ 166.76, 157.01, 155.98, 147.98, 141.83, 131.03, 130.89, 129.76, 129.38, 128.17, 126.91, 125.39, 123.05, 120.78, 120.65, 112.00, 55.69, 54.90, 51.20, 46.59, 37.06, 11.53. C₂₃H₂₇N₃O₂, HRMS (ESI): m/z (M+H⁺): 378.2182 (calculated), 378.2174 (found).

### N-[2-(diethylamino)ethyl]-2-(3-methoxyphenyl)quinoline-4-carboxamide (61)

Yield: 77%. ¹H NMR (400 MHz, DMSO-d₆) δ 8.81 (t, J=5.7 Hz, 1H), 8.26 (ddd, J=8.4, 1.4, 0.6 Hz, 1H), 8.19-8.02 (m, 2H), 7.92-7.76 (m, 3H), 7.63 (ddd, J=8.2, 6.8, 1.3 Hz, 1H), 7.49 (ddd, J=8.1, 7.5, 0.5 Hz, 1H), 7.10 (ddd, J=8.2, 2.6, 1.0 Hz, 1H), 3.89 (s, 3H), 3.47 (q, J=6.5 Hz, 2H), 2.78-2.66 (m, 2H), 2.66-2.53 (m, 4H), 1.03 (t, J=7.1 Hz, 6H). ¹³C NMR (101 MHz, DMSO) δ 166.58, 159.80, 155.52, 147.84, 143.30, 139.74, 130.12, 129.98, 129.50, 127.02, 125.52, 123.49, 119.64, 116.71, 115.59, 112.43, 55.29, 51.36, 46.64, 37.23, 11.62. C₂₃H₂₇N₃O₂, HRMS (ESI): m/z (M+H⁺): 378.2182 (calculated), 378.2174 (found).

### N-[2-(diethylamino)ethyl]-2-(4-methoxyphenyl)quinoline-4-carboxamide (6m)

Yield: 81%. ¹H NMR (400 MHz, DMSO-d₆) δ 8.80-8.78 (m, 1H), 8.37-8.25 (m, 2H), 8.23 (ddd, J=8.4, 1.5, 0.6 Hz, 1H), 8.11-7.99 (m, 2H), 7.78 (ddd, J=8.4, 6.8, 1.4 Hz, 1H), 7.58 (ddd, J=8.3, 6.9, 1.3 Hz, 1H), 7.18-7.04 (m, 2H), 3.85 (s, 3H), 3.48 (q, J=6.5 Hz, 2H), 2.83-2.2.78 (m, 2H), 2.68-2.63 (m, 4H), 1.04 (t, J=7.1 Hz, 6H). ¹³C NMR (101 MHz, DMSO) δ 167.19, 161.30, 155.87, 148.40, 143.61, 131.14, 130.48, 129.73, 129.17, 126.98, 125.96, 123.55, 116.54, 114.74, 55.79, 51.79, 49.06, 47.12, 12.00. C₂₃H₂₇N₃O₂, HRMS (ESI): m/z (M+H⁺): 378.2182 (calculated), 378.2174 (found).

### N-[2-(diethylamino)ethyl]-2-[4-(methoxymethyl)phenyl]quinoline-4-carboxamide (6n)

Yield: 71%. ¹H NMR (400 MHz, DMSO-d₆) δ 8.81 (t, J=5.7 Hz, 1H), 8.39-8.21 (m, 3H), 8.17-8.05 (m, 2H), 7.81 (ddd, J=8.4, 6.8, 1.4 Hz, 1H), 7.62 (ddd, J=8.3, 6.8, 1.3 Hz, 1H), 7.57-7.43 (m, 2H), 4.51 (s, 2H), 3.54-3.41 (m, 2H), 3.35 (s, 3H), 2.76-2.67 (m, 2H), 2.66-2.56 (m, 4H), 1.03 (t, J=7.1 Hz, 6H). ¹³C NMR (101 MHz, DMSO) δ 166.61, 155.49, 147.93, 143.31, 140.18, 137.33, 130.12, 129.44, 127.83, 127.15, 126.94, 125.53, 123.40, 116.48, 73.21, 57.66, 51.36, 46.64, 37.22, 11.60. C₂₄H₂₉N₃O₂, HRMS (ESI): m/z (M+H⁺): 392.2338 (calculated), 392.2329 (found).

### N-[2-(diethylamino)ethyl]-2-{4-[(morpholin-4-yl)methyl]phenyl}quinoline-4-carboxamide (6o)

Yield: 77%. ¹H NMR (400 MHz, DMSO-d₆) δ 8.85-8.82 (m, 1H), 8.36-8.20 (m, 3H), 8.15-8.05 (m, 2H), 7.81 (ddd, J=8.4, 6.8, 1.4 Hz, 1H), 7.62 (ddd, J=8.3, 6.9, 1.3 Hz, 1H), 7.56-7.42 (m, 2H), 3.60 (t, J=4.6 Hz, 4H), 3.55 (s, 2H), 3.53-3.38 (m, 2H), 2.80-2.69 (m, 2H), 2.69-2.54 (m, 4H), 2.45-2.31 (m, 4H), 1.04 (t, J=7.1 Hz, 6H). ¹³C NMR (101 MHz, DMSO) δ 167.11, 156.12, 148.42, 143.68, 140.28, 137.54, 130.58, 129.90, 129.85, 127.62, 127.37, 126.00, 123.84, 117.02, 66.68, 62.57, 53.69, 51.77, 47.11, 11.99. C₂₇H₃₄N₄O₂, HRMS (ESI): m/z (M+H⁺): 447.2760 (calculated), 447.2751 (found).

### N-[2-(diethylamino)ethyl]-2-[2-(dimethylamino)pyrimidin-5-yl]quinoline-4-carboxamide (6p)

Yield: 77%. ¹H NMR (400 MHz, CDCl₃) δ 9.07 (s, 2H), 8.16 (ddd, J=8.3, 1.4, 0.6 Hz, 1H), 8.09 (ddd, J=8.5, 1.3, 0.7 Hz, 1H), 7.73 (s, 1H), 7.70 (ddd, J=8.4, 6.9, 1.4 Hz, 1H), 7.49 (ddd, J=8.3, 6.9, 1.3 Hz, 1H), 6.98-6.91 (m, 1H), 3.61 (dt, J=6.2, 5.2 Hz, 2H), 3.27 (s, 6H), 2.80-2.68 (m, 2H), 2.58 (q, J=7.1 Hz, 4H), 1.03 (t, J=7.1 Hz, 6H). ¹³C NMR (101 MHz, CDCl₃) δ 167.49, 162.29, 156.86, 153.20, 148.93, 143.28, 130.27, 129.89, 126.86, 125.17, 123.25, 120.01, 114.83, 51.37, 46.81, 37.58, 37.42, 11.93. C₂₂H₂₈N₆O, HRMS (ESI): m/z (M+H⁺): 393.2403 (calculated), 393.2377 (found).

### N-[2-(diethylamino)ethyl]-2-(2-methoxypyrimidin-5-yl)quinoline-4-carboxamide (6q)

Yield: 79%. ¹H NMR (400 MHz, DMSO-d₆) δ 9.45 (s, 2H), 8.82-8.80 (m, 1H), 8.33-8.17 (m, 2H), 8.11 (ddd, J=8.5, 1.3, 0.6 Hz, 1H), 7.82 (ddd, J=8.4, 6.9, 1.4 Hz, 1H), 7.64 (ddd, J=8.3, 6.9, 1.3 Hz, 1H), 4.02 (s, 3H), 3.64-3.36 (m, 2H), 2.75-2.73 (m, 2H), 2.68-2.63 (m, 4H), 1.04 (t, J=7.1 Hz, 6H). ¹³C NMR (101 MHz, DMSO) δ 166.84, 166.11, 158.84, 151.95, 148.28, 144.16, 130.85, 129.84, 127.74, 126.28, 126.05, 123.95, 116.48, 55.51, 51.77, 47.07, 37.58, 11.89. C₂₁H₂₅N₅O₂, HRMS (ESI): m/z (M+H⁺): 380.2087 (calculated), 380.2077 (found).

### N-[2-(diethylamino)ethyl]-2 [(E)-2-phenylethenyl]quinoline-4-carboxamide (6r)

Yield: 71%. ¹H NMR (400 MHz, DMSO-d₆) δ 8.82-8.74 (m, 1H), 8.18 (ddd, J=8.3, 1.5, 0.6 Hz, 1H), 8.04 (ddd, J=8.5, 1.3, 0.6 Hz, 1H), 7.90 (d, J=17.0 Hz, 2H), 7.82-7.68 (m, 3H), 7.59 (ddd, J=8.2, 6.9, 1.3 Hz, 1H), 7.52 (d, J=16.4 Hz, 1H), 7.47-7.40 (m, 2H), 7.40-7.32 (m, 1H), 3.48 (q, J=6.5 Hz, 2H), 2.82-2.71 (m, 2H), 2.70-2.56 (m, 4H), 1.05 (t, J=7.1 Hz, 6H). ¹³C NMR (101 MHz, DMSO) δ 166.67, 155.32, 147.96, 142.95, 136.09, 134.72, 130.05, 129.03, 128.90, 128.86, 128.30, 127.31, 126.69, 125.50, 123.38, 117.38, 51.28, 46.60, 37.03, 11.44. C₂₄H₂₇N₃O, HRMS (ESI): m/z (M+H⁺): 374.2232 (calculated), 374.2228 (found).

### N-[2-(diethylamino)ethyl]-2-(2,3-dihydro-1-benzofuran-5-yl)quinoline-4-carboxamide (6s)

Yield: 86%. ¹H NMR (400 MHz, DMSO-d₆) δ 8.79 (t, J=5.8 Hz, 1H), 8.32-8.16 (m, 2H), 8.16-7.98 (m, 3H), 7.77 (ddd, J=8.4, 6.9, 1.5 Hz, 1H), 7.57 (ddd, J=8.3, 6.8, 1.3 Hz, 1H), 6.93 (d, J=8.3 Hz, 1H), 4.63 (t, J=8.7 Hz, 2H), 3.47 (q, J=6.5 Hz, 2H), 3.38-3.22 (m, 2H), 2.78-2.69 (m, 2H), 2.69-2.54 (m, 4H), 1.04 (t, J=7.1 Hz, 6H). ¹³C NMR (101 MHz, DMSO) δ 166.73, 161.49, 155.68, 147.93, 142.99, 130.81, 129.96, 129.17, 128.31, 127.69, 126.37, 125.47, 124.14, 123.02, 116.15, 109.11, 71.58, 51.32, 46.65, 37.13, 28.83, 11.53. C₂₄H₂₇N₃O₂, HRMS (ESI): m/z (M+H⁺): 390.2182 (calculated), 390.2172 (found).

### N-[2-(diethylamino)ethyl]-2-(2,3-dihydro-1,4-benzodioxin-6-yl)quinoline-4-carboxamide (6t)

Yield: 91%. ¹H NMR (400 MHz, DMSO-d₆) δ 8.81 (t, J=5.6 Hz, 1H), 8.23 (ddd, J=8.4, 1.5, 0.7 Hz, 1H), 8.12-7.98 (m, 2H), 7.88-7.70 (m, 3H), 7.58 (ddd, J=8.3, 6.8, 1.3 Hz, 1H), 7.03 (d, J=8.4 Hz, 1H), 4.33 (s, 4H), 3.49 (q, J=6.5 Hz, 2H), 2.80-2.72 (m, 2H), 2.72-2.56 (m, 4H), 1.05 (t, J=7.1 Hz, 6H). ¹³C NMR (101 MHz, DMSO) δ 166.66, 155.06, 147.87, 145.21, 143.72, 142.92, 131.53, 130.01, 129.30, 126.62, 125.48, 123.16, 120.45, 117.42, 116.19, 115.86, 64.37, 64.10, 54.90, 51.20, 46.64, 36.97, 11.36. C₂₄H₂₇N₃O₃, HRMS (ESI): m/z (M+H⁺): 406.2131 (calculated), 406.2118 (found).

### 2-{[1,1′-biphenyl]-4-yl}-N-[2-(diethylamino)ethyl]quinoline-4-carboxamide (6u)

Yield: 77%. ¹H NMR (400 MHz, DMSO-d₆) δ 8.85 (t, J=5.8 Hz, 1H), 8.48-8.36 (m, 2H), 8.28 (dd, J=8.5, 1.4 Hz, 1H), 8.20 (s, 1H), 8.15 (dd, J=8.6, 1.2 Hz, 1H), 7.93-7.86 (m, 2H), 7.86-7.73 (m, 3H), 7.64 (ddd, J=8.2, 6.8, 1.3 Hz, 1H), 7.51 (dd, J=8.4, 6.9 Hz, 2H), 7.46-7.34 (m, 1H), 3.50 (q, J=6.5 Hz, 2H), 2.81-2.71 (m, 2H), 2.71-2.54 (m, 4H), 1.05 (t, J=7.1 Hz, 6H). ¹³C NMR (101 MHz, DMSO) δ 166.64, 155.25, 147.99, 143.28, 141.40, 139.32, 137.17, 130.16, 129.48, 129.01, 127.87, 127.80, 127.10, 126.99, 126.72, 125.55, 123.45, 116.50, 51.31, 46.65, 38.21, 11.51. C₂₈H₂₉N₃O, HRMS (ESI): m/z (M+H⁺): 424.2389 (calculated), 424.2379 (found).

### 2-(propan-2-yloxy)quinoline-4-carboxylic Acid (7)

Compound 7 was synthesized using synthesis route 1 method 1. Yield: 84%. ¹H NMR (400 MHz, DMSO-d₆) δ 8.49 (ddd, J=8.2, 1.5, 0.6 Hz, 1H), 7.74 (ddd, J=8.3, 1.4, 0.6 Hz, 1H), 7.62 (ddd, J=8.4, 6.9, 1.5 Hz, 1H), 7.40 (ddd, J=8.3, 6.9, 1.3 Hz, 1H), 7.11 (s, 1H), 5.47 (p, J=6.2 Hz, 1H), 1.35 (d, J=6.2 Hz, 6H). ¹³C NMR (101 MHz, DMSO) δ 168.92, 161.27, 147.24, 145.76, 129.89, 127.44, 126.85, 124.50, 122.38, 113.17, 68.10, 22.27. C₁₃H₁₃NO₃, EI-MS: m/z (M−H⁺): 230.3 (calculated), 230.3 (found).

### N-[2-(dimethylamino)ethyl]-2-(propan-2-yloxy)quinoline-4-carboxamide (8a)

Yield: 80%. ¹H NMR (400 MHz, DMSO-d₆) δ 8.78-8.58 (m, 1H), 8.06 (ddd, J=8.3, 1.5, 0.6 Hz, 1H), 7.79 (ddd, J=8.4, 1.3, 0.6 Hz, 1H), 7.68 (ddd, J=8.4, 6.9, 1.5 Hz, 1H), 7.45 (ddd, J=8.3, 6.9, 1.3 Hz, 1H), 5.59-5.43 (m, 1H), 3.43 (td, J=6.7, 5.7 Hz, 2H), 2.57-2.43 (m, 2H), 2.25 (s, 6H), 1.38 (d, J=6.2 Hz, 6H). ¹³C NMR (101 MHz, DMSO) δ 166.09, 160.46, 146.46, 145.65, 129.97, 127.08, 125.49, 124.40, 121.32, 111.33, 67.88, 57.86, 45.09, 37.13, 21.79. C₁₇H₂₃N₃O₂, HRMS (ESI): m/z (M+H⁺): 302.1869 (calculated), 302.1863 (found).

### N-(2-aminoethyl)-2-(propan-2-yloxy)quinoline-4-carboxamide (8b)

Compound 8b was synthesized by first coupling intermediate 7 with N-Boc-ethylenediamine, followed by TFA deprotection. Yield: 72%. (TFA salt). ¹H NMR (400 MHz, DMSO-d₆) δ 8.92 (t, J=5.6 Hz, 1H), 8.18-7.96 (m, 4H), 7.78 (d, J=8.2 Hz, 1H), 7.67 (t, J=7.7 Hz, 1H), 7.47-7.37 (m, 1H), 7.22 (s, 1H), 5.58-5.40 (m, 1H), 3.65-3.49 (m, 2H), 3.13-2.97 (m, 2H), 1.37 (d, J=6.2 Hz, 6H). ¹³C NMR (101 MHz, DMSO) δ 166.74, 160.55, 146.55, 144.59, 130.01, 127.15, 125.56, 124.45, 121.21, 112.28, 67.92, 38.42, 37.08, 21.79. C₁₅H₁₉N₃O₂, HRMS (ESI): m/z (M+H⁺): 274.1556 (calculated), 274.1550 (found).

### N-[3-(dimethylamino)propyl]-2-(propan-2-yloxy)quinoline-4-carboxamide (8c)

Yield: 93%. ¹H NMR (400 MHz, DMSO-d₆) δ 8.74 (t, J=5.6 Hz, 1H), 7.99 (ddd, J=8.2, 1.5, 0.6 Hz, 1H), 7.77 (ddd, J=8.4, 1.3, 0.6 Hz, 1H), 7.67 (ddd, J=8.4, 6.9, 1.5 Hz, 1H), 7.44 (ddd, J=8.2, 6.9, 1.3 Hz, 1H), 6.94 (s, 1H), 5.51 (p, J=6.2 Hz, 1H), 3.33 (td, J=6.9, 5.6 Hz, 2H), 2.46-2.36 (m, 2H), 2.23 (s, 6H), 1.72 (dq, J=8.5, 7.0 Hz, 2H), 1.37 (d, J=6.2 Hz, 6H). ¹³C NMR (101 MHz, DMSO) δ 166.03, 160.46, 146.43, 145.61, 129.96, 127.09, 125.34, 124.40, 121.29, 111.32, 67.87, 56.35, 44.73, 37.21, 26.51, 21.78. C₁₈H₂₅N₃O₂, HRMS (ESI): m/z (M−H^{f}): 316.2025 (calculated), 316.2017 (found).

### N-[2-(morpholin-4-yl)ethyl]-2-(propan-2-yloxy)quinoline-4-carboxamide (8d)

Yield: 70%. ¹H NMR (400 MHz, DMSO-d₆) δ 8.71 (t, J=5.8 Hz, 1H), 8.23-8.10 (m, 1H), 7.84 (dd, J=8.4, 1.2 Hz, 1H), 7.73 (ddd, J=8.4, 6.8, 1.5 Hz, 1H), 7.49 (ddd, J=8.2, 6.8, 1.3 Hz, 1H), 6.97 (s, 1H), 5.57 (p, J=6.2 Hz, 1H), 3.66 (t, J=4.6 Hz, 4H), 3.50 (q, J=6.3 Hz, 2H), 2.77-2.70 (m, 3H), 2.60-2.53 (m, 3H), 1.43 (d, J=6.2 Hz, 6H). ¹³C NMR (101 MHz, DMSO) δ 166.06, 160.47, 146.43, 145.92, 129.97, 127.05, 125.58, 124.30, 121.33, 111.15, 67.86, 66.27, 57.15, 53.20, 38.21, 36.16, 21.77. C₁₉H₂₅N₃O₃, HRMS (ESI): m/z (M−H⁺): 344.1974 (calculated), 344.1967 (found).

### 2-(propan-2-yloxy)-N-[2-(pyrrolidin-1-yl)ethyl]quinoline-4-carboxamide (8e)

Yield: 72%. ¹H NMR (400 MHz, DMSO-d₆) δ 8.84 (t, J=5.7 Hz, 1H), 8.08 (ddd, J=8.3, 1.5, 0.6 Hz, 1H), 7.79 (ddd, J=8.3, 1.3, 0.6 Hz, 1H), 7.69 (ddd, J=8.4, 6.9, 1.5 Hz, 1H), 7.44 (ddd, J=8.2, 6.9, 1.3 Hz, 1H), 7.03 (s, 1H), 5.52 (p, J=6.2 Hz, 1H), 3.54 (q, J=6.3 Hz, 2H), 2.92 (t, J=6.5 Hz, 2H), 2.86-2.84 (m, 4H), 1.90-1.71 (m, 4H), 1.38 (d, J=6.2 Hz, 6H). ¹³C NMR (101 MHz, DMSO) δ 166.71, 160.92, 146.93, 145.75, 130.45, 127.54, 125.97, 124.84, 121.73, 112.01, 68.36, 54.37, 53.88, 49.05, 37.78, 23.44, 22.24. C₁₉H₂₅N₃O₂, HRMS (ESI): m/z (M+H⁺): 328.2025 (calculated), 328.2018 (found).

### N-[2-(4-methylpiperazin-1-yl)ethyl]-2-(propan-2-yloxy)quinoline-4-carboxamide (8f)

Yield: 84%. ¹H NMR (400 MHz, DMSO-d₆) δ 8.68 (t, J=5.7 Hz, 1H), 8.07 (dd, J=8.3, 1.4 Hz, 1H), 7.78 (dd, J=8.4, 1.3 Hz, 1H), 7.68 (ddd, J=8.4, 6.8, 1.5 Hz, 1H), 7.44 (ddd, J=8.2, 6.9, 1.3 Hz, 1H), 6.92 (s, 1H), 5.50 (p, J=6.2 Hz, 1H), 3.44 (t, J=6.2 Hz, 2H), 2.79 (s, 4H), 2.70-2.52 (m, 5H), 2.52-2.41 (m, 4H), 1.37 (d, J=6.2 Hz, 6H). ¹³C NMR (101 MHz, DMSO) δ 166.10, 160.47, 146.43, 145.76, 130.00, 127.07, 125.56, 124.38, 121.29, 111.23, 67.89, 56.15, 53.58, 50.83, 43.85, 36.35, 21.79. C₂₀H₂₈N₁₄O₂, HRMS (ESI): m/z (M+H⁺): 357.2291 (calculated), 357.2284 (found).

### N-(1-methylpiperidin-4-yl)-2-(propan-2-yloxy)quinoline-4-carboxamide (8g)

Yield: 90%. ¹H NMR (400 MHz, DMSO-d₆) δ 8.70 (d, J=7.6 Hz, 1H), 7.98 (ddd, J=8.2, 1.5, 0.6 Hz, 1H), 7.78 (ddd, J=8.4, 1.3, 0.6 Hz, 1H), 7.67 (ddd, J=8.4, 6.9, 1.5 Hz, 1H), 7.44 (ddd, J=8.2, 6.9, 1.3 Hz, 1H), 6.93 (s, 1H), 5.51 (p, J=6.2 Hz, 1H), 3.93-3.81 (m, 1H), 3.00-2.82 (m, 2H), 2.30 (s, 3H), 2.30-2.20 (m, 2H), 1.95-1.85 (m, 2H), 1.71-1.55 (m, 2H), 1.37 (d, J=6.2 Hz, 6H). ¹³C NMR (101 MHz, DMSO) δ 165.50, 160.42, 146.41, 145.47, 129.94, 127.10, 125.26, 124.41, 121.29, 111.43, 67.84, 53.52, 45.58, 45.02, 30.45, 21.78. C₁₉H₂₅N₃O₂, HRMS (ESI): m/z (M+H⁺): 328.2025 (calculated), 328.2018 (found).

### 4-(4-methylquinoline-1-carbonyl)-2-(propan-2-yloxy)quinoline (8h)

Yield: 78%. ¹H NMR (400 MHz, DMSO-d₆) δ 7.80 (ddd, J=8.4, 1.3, 0.6 Hz, 1H), 7.70 (ddd, J=8.3, 6.9, 1.5 Hz, 1H), 7.64 (ddd, J=8.1, 1.5, 0.6 Hz, 1H), 7.47 (ddd, J=8.2, 6.9, 1.3 Hz, 1H), 6.88 (s, 1H), 5.50 (p, J=6.2 Hz, 1H), 3.89-3.85 (m, 1H), 3.71-3.55 (m, 1H), 3.20-3.07 (m, 2H), 2.50-2.46 (m, 1H), 2.42-2.38 (m, 1H), 2.27-2.23 (m, 1H), 2.19 (s, 3H), 2.13-2.00 (m, 1H), 1.38 (t, J=5.7 Hz, 6H). ¹³C NMR (101 MHz, DMSO) δ 165.56, 161.06, 146.72, 146.10, 130.75, 127.76, 125.20, 125.08, 121.23, 110.58, 68.47, 55.19, 54.63, 46.88, 45.99, 41.46, 38.70, 22.24. C₁₈H₂₃N₃O₂, HRMS (ESI): m/z (M+H⁺): 314.1869 (calculated), 314.1866 (found).

### N-[2-(dimethylamino)ethyl]-2-phenylquinoline-4-carboxamide (10a)

Yield: 93%. ¹H NMR (400 MHz, DMSO-d₆) δ 8.82 (t, J=5.7 Hz, 1H), 8.39-8.29 (m, 2H), 8.25 (ddd, J=8.4, 1.5, 0.6 Hz, 1H), 8.20-8.07 (m, 2H), 7.83 (ddd, J=8.4, 6.8, 1.5 Hz, 1H), 7.65 (ddd, J=8.2, 6.8, 1.3 Hz, 1H), 7.63-7.45 (m, 3H), 3.50 (td, J=6.7, 5.7 Hz, 2H), 2.55-2.53 (m, 2H), 2.26 (s, 6H). ¹³C NMR (101 MHz, DMSO) δ 167.08, 156.21, 148.38, 143.84, 138.72, 130.60, 130.32, 129.96, 129.36, 127.73, 127.52, 125.94, 123.90, 117.01, 58.50, 45.70, 37.86. C₂₀H₂₁N₃O, HRMS (ESI): m/z (M+H⁺): 320.1763 (calculated), 320.1758 (found).

### N-[3-(diethylamino)propyl]-2-phenylquinoline-4-carboxamide (10b)

¹H NMR (400 MHz, DMSO-d₆) δ 8.91 (t, J=5.6 Hz, 1H), 8.40-8.27 (m, 2H), 8.19 (dd, J=8.4, 1.4 Hz, 1H), 8.17-8.07 (m, 2H), 7.82 (ddd, J=8.4, 6.8, 1.5 Hz, 1H), 7.64 (ddd, J=8.2, 6.8, 1.3 Hz, 1H), 7.60-7.42 (m, 3H), 3.42 (q, J=6.5 Hz, 2H), 2.66-2.52 (m, 6H), 1.77 (p, J=7.0 Hz, 2H), 0.99 (t, J=7.1 Hz, 6H). ¹³C NMR (101 MHz, DMSO) δ 166.55, 155.76, 147.92, 143.28, 138.23, 130.12, 129.85, 129.52, 128.87, 127.25, 127.03, 125.31, 123.40, 116.56, 49.76, 46.27, 37.62, 26.07, 11.22. C₂₃H₂₇N₃O, HRMS (ESI): m/z (M+H⁺): 362.2232 (calculated), 362.2221 (found).

### N-{1-azabicyclo[2.2.2]octan-3-yl}-2-phenylquinoline-4-carboxamide (10e)

Yield: 81%. ¹H NMR (400 MHz, DMSO-d₆) δ 8.89 (d, J=7.0 Hz, 1H), 8.39-8.27 (m, 2H), 8.18-8.04 (m, 3H), 7.82 (ddd, J=8.4, 6.8, 1.5 Hz, 1H), 7.64 (ddd, J=8.2, 6.8, 1.3 Hz, 1H), 7.61-7.43 (m, 3H), 4.15-4.01 (m, 1H), 3.26-3.13 (m, 1H), 2.90-2.77 (m, 1H), 2.77-2.58 (m, 4H), 2.09-1.98 (m, 1H), 1.90-1.75 (m, 1H), 1.70-1.56 (m, 2H), 1.43-1.29 (m, 1H). ¹³C NMR (101 MHz, DMSO) δ 166.71, 155.73, 147.82, 143.52, 138.24, 130.11, 129.85, 129.51, 128.89, 127.30, 127.09, 125.17, 123.47, 116.60, 53.87, 47.16, 46.80, 46.33, 25.81, 25.62, 19.96. C₂₃H₂₃N₃O, HRMS (ESI): m/z (M+H⁺): 358.1919 (calculated), 358.1913 (found).

### 2-phenylquinoline-4-carbohydrazide (10d)

Yield: 82%. Compound 10d was synthesized through the ester intermediate. Briefly, to a solution of ethyl 2-phenylquinoline-4-carboxylate (1 mmol) in ethanol was added hydrazine monohydrate (2 mmol). The mixture was heated under reflux to 130° C. in a sealed tube for 12 hours. Solvent was removed in vacuo and the product was purified by flash column chromatography (5-15% CH₃OH/CH₂Cl₂). ¹H NMR (400 MHz, DMSO-d₆) δ 10.03 (s, 1H), 8.35-8.27 (m, 2H), 8.25 (dd, J=8.4, 1.4 Hz, 1H), 8.17-8.08 (m, 2H), 7.82 (ddd, J=8.4, 6.8, 1.5 Hz, 1H), 7.64 (ddd, J=8.3, 6.8, 1.3 Hz, 1H), 7.61-7.45 (m, 3H), 4.73 (s, 2H). ¹³C NMR (101 MHz, DMSO) δ 165.87, 155.74, 147.90, 141.69, 138.23, 130.18, 129.87, 129.52, 128.91, 127.28, 127.07, 125.41, 123.61, 116.93. C₁₆H₁₃N₃O, HRMS (ESI): m/z (M+H⁺): 264.1137 (calculated), 264.1129 (found).

### N-carbamimidoyl-2-phenylquinoline-4-carboxamide (10e)

Compound 10e was synthesized through the acid chloride intermediate. Briefly, to a solution of intermediate 9 (1 mmol) in dichloromethane was added four drops of DMF, followed by oxalyl chloride (1.2 mmol). The mixture was stirred at room temperature for 1 hour and the solvent was removed in vacuo. THF was added to dissolve the acid chloride intermediate. In a separate round bottom flask, to a NaOH aqueous solution (15 eq) was added guanidine HCl (15 mmol). After 30 minutes, the acid chloride solution in THF was added dropwise to the aqueous solution of neutralized guanidine. The resulting mixture was stirred at room temperature overnight. THF was removed in vacuo and the resulting solution was extracted with dichloromethane. The DCM layer was separated and acidified with HCl. Final product was purified by flash column chromatography (10-20% CH₃OH/CH₂Cl₂). Yield: 89% (HCl salt). ¹H NMR (400 MHz, DMSO-d₆) δ 12.92 (s, 1H), 8.92 (br s, 2H), 8.74 (br s, 2H), 8.59 (s, 1H), 8.48-8.37 (m, 2H), 8.31 (dd, J=8.5, 1.2 Hz, 1H), 8.20 (dd, J=8.5, 1.1 Hz, 1H), 7.89 (ddd, J=8.3, 6.8, 1.4 Hz, 1H), 7.73 (ddd, J=8.3, 6.9, 1.3 Hz, 1H), 7.68-7.47 (m, 3H). ¹³C NMR (101 MHz, DMSO) δ 167.43, 155.83, 155.66, 147.70, 138.25, 137.61, 130.85, 130.33, 129.41, 128.96, 128.16, 127.78, 124.89, 122.53, 119.08. C₁₇H₁₄N₄O, HRMS (ESI): m/z (M+H⁺): 291.1246 (calculated), 291.1239 (found).

### N-[4-(dimethylamino)phenyl]-2-phenylquinoline-4-carboxamide (10f)

Yield: 89%. ¹H NMR (400 MHz, CDCl₃-d) δ 8.26 (s, 1H), 8.14-8.09 (m, 1H), 8.09-7.98 (m, 3H), 7.76 (s, 1H), 7.72-7.65 (m, 1H), 7.65-7.58 (m, 2H), 7.52-7.39 (m, 4H), 6.86-6.74 (m, 2H), 2.99 (s, 6H). ¹³C NMR (101 MHz, CDCl₃) δ 165.35, 156.68, 148.57, 148.55, 143.23, 138.55, 130.31, 129.89, 129.78, 128.99, 127.52, 127.50, 127.40, 125.20, 123.33, 122.06, 116.39, 113.07, 40.97. C₂₄H₂₁N₃O, HRMS (ESI): m/z (M+H⁺): 368.1763 (calculated), 368.1750 (found).

### 2-phenyl-N-[2-(pyrrolidin-1-yl)ethyl]quinoline-4-carboxamide (10g)

¹HNMR (400 MHz, DMSO-d₆) δ 8.91 (s, 1H), 8.30 (ddd, J=21.3, 8.5, 1.6 Hz, 3H), 8.21-8.09 (m, 2H), 7.83 (ddd, J=8.4, 6.8, 1.4 Hz, 1H), 7.72-7.46 (m, 4H), 3.56 (q, J=6.4 Hz, 2H), 2.79 (t, J=6.8 Hz, 2H), 2.67 (s, 5H), 1.76 (p, J=3.0 Hz, 4H). ¹³C NMR (101 MHz, DMSO) δ 166.66, 155.73, 147.90, 143.28, 138.22, 130.11, 129.83, 129.47, 128.87, 127.25, 126.98, 125.50, 123.42, 116.58, 54.38, 53.52, 38.22, 38.14, 23.15. C₂₂H₂₃N₃O, HRMS (ESI): m/z (M+H⁺): 346.1919 (calculated), 346.1914 (found).

### N-[3-(dimethylamino)propyl]-2-phenylquinoline-4-carboxamide (10h)

Yield: 87%. ¹H NMR (400 MHz, DMSO-d₆) δ 8.88 (t, J=5.6 Hz, 1H), 8.37-8.25 (m, 2H), 8.19 (dd, J=8.4, 1.4 Hz, 1H), 8.16-8.03 (m, 2H), 7.82 (ddd, J=8.4, 6.8, 1.4 Hz, 1H), 7.64 (ddd, J=8.2, 6.8, 1.3 Hz, 1H), 7.61-7.43 (m, 3H), 3.53-3.32 (m, 2H), 2.38 (t, J=7.1 Hz, 2H), 2.20 (s, 6H), 1.77 (p, J=7.1 Hz, 2H). ¹³C NMR (101 MHz, DMSO) δ 166.54, 155.77, 147.91, 143.30, 138.24, 130.12, 129.84, 129.51, 128.88, 127.26, 127.05, 125.33, 123.42, 116.56, 56.63, 45.02, 37.52, 26.81. C₂₁H₂₃N₃O, HRMS (ESI): m/z (M+H⁺): 334.1919 (calculated), 334.1913 (found).

### N-[2-(dimethylamino)ethyl]-2-(thiophen-2-yl)quinoline-4-carboxamide (12a)

Yield: 88%. ¹H NMR (400 MHz, DMSO-d₆) δ 8.79 (t, J=5.7 Hz, 1H), 8.14 (ddd, J=8.4, 1.5, 0.6 Hz, 1H), 8.10 (s, 1H), 8.06 (dd, J=3.8, 1.1 Hz, 1H), 8.01 (ddd, J=8.5, 1.3, 0.6 Hz, 1H), 7.85-7.70 (m, 2H), 7.59 (ddd, J=8.2, 6.8, 1.3 Hz, 1H), 7.24 (dd, J=5.0, 3.7 Hz, 1H), 3.48 (td, J=6.7, 5.7 Hz, 2H), 2.57-2.50 (m, 2H), 2.26 (s, 6H). ¹³C NMR (101 MHz, DMSO) δ 166.43, 151.58, 147.59, 144.27, 143.54, 130.32, 129.99, 128.76, 128.60, 127.58, 126.74, 125.50, 123.36, 115.35, 57.94, 45.15, 37.28. C₁₈H₁₉N₃OS, HRMS (ESI): m/z (M+H⁺): 326.1327 (calculated), 326.1324 (found).

### N-[3-(dimethylamino)propyl]-2-(thiophen-2-yl)quinoline-4-carboxamide (12b)

Yield: 92%. ¹H NMR (400 MHz, DMSO-d₆) δ 8.91 (t, J=5.6 Hz, 1H), 8.12 (s, 1H), 8.11-8.04 (m, 2H), 8.01 (ddd, J=8.4, 1.3, 0.6 Hz, 1H), 7.82-7.69 (m, 2H), 7.60 (ddd, J=8.3, 6.9, 1.3 Hz, 1H), 7.24 (dd, J=5.0, 3.7 Hz, 1H), 3.40 (td, J=7.0, 5.6 Hz, 2H), 2.46 (t, J=7.2 Hz, 2H), 2.26 (s, 6H), 1.86-1.68 (m, 2H). ¹³C NMR (101 MHz, DMSO) δ 166.38, 151.63, 147.61, 144.27, 143.40, 130.32, 130.01, 128.80, 128.60, 127.65, 126.77, 125.36, 123.36, 115.43, 56.37, 44.69, 37.38, 26.50. C₁₉H₂₁N₃OS, HRMS (ESI): m/z (M+H⁺): 340.1484 (calculated), 340.1472 (found).

### N-[2-(pyrrolidin-1-yl)ethyl]-2-(thiophen-2-yl)quinoline-4-carboxamide (12c)

Yield: 88%. ¹H NMR (400 MHz, DMSO-d₆) δ 8.84 (t, J=5.7 Hz, 1H), 8.25-8.16 (m, 1H), 8.14 (s, 1H), 8.07 (dd, J=3.8, 1.1 Hz, 1H), 8.01 (ddd, J=8.4, 1.3, 0.6 Hz, 1H), 7.84-7.70 (m, 2H), 7.58 (ddd, J=8.3, 6.9, 1.3 Hz, 1H), 7.24 (dd, J=5.0, 3.7 Hz, 1H), 3.52 (q, J=6.6 Hz, 2H), 2.73 (t, J=6.7 Hz, 2H), 2.66-2.54 (m, 4H), 1.74 (p, J=3.3 Hz, 4H). ¹³C NMR (101 MHz, DMSO) δ 166.45, 151.61, 147.60, 144.29, 143.57, 130.32, 129.99, 128.75, 128.60, 127.60, 126.68, 125.57, 123.38, 115.37, 54.42, 53.53, 38.24, 23.19. C₂₄H₂₁N₃OS, HRMS (ESI): m/z (M+H⁺): 352.1484 (calculated), 352.1473 (found).

### N-[3-(1H-imidazol-1-yl)propyl]-2-(thiophen-2-yl)quinoline-4-carboxamide (12d)

Yield: 82%. ¹H NMR (400 MHz, DMSO-d₆) δ 8.91 (t, J=5.5 Hz, 1H), 8.16-8.04 (m, 3H), 8.01 (dd, J=8.6, 1.2 Hz, 1H), 7.84-7.73 (m, 2H), 7.69 (d, J=1.1 Hz, 1H), 7.60 (ddd, J=8.2, 6.9, 1.3 Hz, 1H), 7.31-7.20 (m, 2H), 6.92 (t, J=1.1 Hz, 1H), 4.11 (t, J=6.9 Hz, 2H), 3.41-3.27 (m, 2H), 2.05 (p, J=6.8 Hz, 2H). ¹³C NMR (101 MHz, DMSO) δ 166.59, 151.64, 147.59, 144.25, 143.29, 137.38, 130.38, 130.08, 128.81, 128.61, 128.44, 127.74, 126.87, 125.34, 123.28, 119.41, 115.49, 43.77, 36.51, 30.56. C₂₄H₁₈N₄OS, HRMS (ESI): m/z (M+H⁺): 363.1280 (calculated), 363.1270 (found).

### 2-(thiophen-2-yl)quinoline-4-carbohydrazide (12e)

Yield: 77%. Compound 12e was synthesized by following the same procedure as compound 10e. ¹H NMR (400 MHz, DMSO-d₆) δ 9.98 (s, 1H), 8.13 (dd, J=8.4, 1.4 Hz, 1H), 8.09 (s, 1H), 8.05 (dd, J=3.7, 1.2 Hz, 1H), 8.03-7.96 (m, 1H), 7.82-7.73 (m, 2H), 7.60 (ddd, J=8.3, 6.9, 1.3 Hz, 1H), 7.24 (dd, J=5.0, 3.7 Hz, 1H), 4.71 (s, 2H). ¹³C NMR (101 MHz, DMSO) δ 165.64, 151.55, 147.57, 144.20, 141.88, 130.38, 130.05, 128.79, 128.65, 127.64, 126.77, 125.44, 123.56, 115.79. C₁₄H₁₁N₃OS, HRMS (ESI): m/z (M+H⁺): 270.0701 (calculated), 270.0696 (found).

### 6-chloro-2-phenylquinoline-4-carboxylic Acid (14a)

Yield: 50%. ¹H NMR (400 MHz, DMSO-d₆) δ 8.74 (d, J=2.4 Hz, 1H), 8.49 (s, 1H), 8.30-8.17 (m, 2H), 8.10 (d, J=9.0 Hz, 1H), 7.80 (dd, J=9.0, 2.4 Hz, 1H), 7.62-7.45 (m, 3H). ¹³C NMR (101 MHz, DMSO) δ 167.00, 156.26, 146.91, 137.48, 136.03, 132.38, 131.77, 130.58, 130.16, 128.98, 127.22, 124.32, 124.26, 120.54. C₁₆H₁₀ClNO₂, EI-MS: m/z (M−H⁺): 282.7 (calculated), 282.5 (found).

### 6-methoxy-2-phenylquinoline-4-carboxylic Acid (14b)

Yield: 57%. ¹H NMR (400 MHz, DMSO-d₆) δ 8.44 (s, 1H), 8.30-8.19 (m, 2H), 8.13 (d, J=2.8 Hz, 1H), 8.06 (d, J=9.2 Hz, 1H), 7.64-7.36 (m, 4H), 3.91 (s, 3H). ¹³C NMR (101 MHz, DMSO) δ 167.68, 158.30, 153.16, 144.76, 138.05, 135.41, 131.36, 129.49, 128.92, 126.84, 124.98, 122.45, 119.75, 103.67, 55.44. C₁₇H₁₃NO₃, EI-MS: m/z (M−H⁺): 278.3 (calculated), 278.3 (found).

### 6-chloro-N-[2-(dimethylamino)ethyl]-2-phenylquinoline-4-carboxamide (15a)

Yield: 82%. ¹H NMR (400 MHz, DMSO-d₆) δ 8.91 (t, J=5.7 Hz, 1H), 8.37 (dd, J=2.4, 0.5 Hz, 1H), 8.34-8.26 (m, 2H), 8.21 (s, 1H), 8.13 (dd, J=9.0, 0.5 Hz, 1H), 7.82 (dd, J=9.0, 2.4 Hz, 1H), 7.61-7.49 (m, 3H), 3.52 (q, J=6.4 Hz, 2H), 2.62 (t, J=6.5 Hz, 2H), 2.34 (s, 6H). ¹³C NMR (101 MHz, DMSO) δ 166.59, 156.74, 146.88, 142.67, 138.27, 132.06, 132.03, 131.12, 130.58, 129.40, 127.78, 124.94, 124.61, 118.12, 58.13, 45.32, 37.48. C₂₀H₂₀ClN₃O, HRMS (ESI): m/z (M+H⁺): 354.1373 (calculated), 354.1362 (found).

### 6-chloro-N-[2-(diethylamino)ethyl]-2-phenylquinoline-4-carboxamide (15b)

Yield: 82%. ¹H NMR (400 MHz, CD₃OD-d₄) δ 8.27 (d, J=2.3 Hz, 1H), 8.21-8.10 (m, 2H), 8.10-7.98 (m, 2H), 7.68 (dd, J=9.0, 2.4 Hz, 1H), 7.58-7.42 (m, 3H), 3.70 (t, J=6.7 Hz, 2H), 3.05 (t, J=6.7 Hz, 2H), 2.94 (q, J=7.2 Hz, 4H), 1.23 (t, J=7.2 Hz, 6H). ¹³C NMR (101 MHz, CD₃OD) δ 169.14, 158.31, 147.83, 142.19, 139.23, 133.92, 131.95, 131.82, 130.85, 129.71, 128.36, 125.10, 125.03, 119.03, 52.06, 48.17, 37.24, 10.58. C₂₂H₂₄ClN₃O, HRMS (ESI): m/z (M+H⁺): 382.1686 (calculated), 382.1675 (found).

### N-[2-(dimethylamino)ethyl]-6-methoxy-2-phenylquinoline-4-carboxamide (15c)

Yield: 90%. ¹H NMR (400 MHz, DMSO-d₆) δ 8.80 (t, J=5.7 Hz, 1H), 8.34-8.21 (m, 2H), 8.08 (s, 1H), 8.04 (d, J=9.2 Hz, 1H), 7.62 (d, J=2.8 Hz, 1H), 7.55 (dd, J=8.2, 6.4 Hz, 2H), 7.49 (tt, J=9.2, 2.1 Hz, 2H), 3.89 (s, 3H), 3.50 (q, J=6.3 Hz, 2H), 2.56 (t, J=6.6 Hz, 2H), 2.27 (s, 6H). ¹³C NMR (101 MHz, DMSO) δ 166.74, 157.66, 153.22, 144.10, 141.74, 138.34, 131.07, 129.40, 128.83, 126.91, 124.50, 122.46, 116.93, 103.45, 57.93, 55.41, 45.11, 37.12. C₂₁H₂₃N₃O₂, HRMS (ESI): m/z (M+H⁺): 350.1869 (calculated), 350.1859 (found).

### N-[2-(diethylamino)ethyl]-6-methoxy-2-phenylquinoline-4-carboxamide (15d)

Yield: 92%. ¹H NMR (400 MHz, DMSO-d₆) δ 8.93-8.80 (m, 1H), 8.35-8.19 (m, 2H), 8.13 (s, 1H), 8.04 (d, J=9.2 Hz, 1H), 7.65 (d, J=2.9 Hz, 1H), 7.60-7.53 (m, 2H), 7.53-7.41 (m, 2H), 3.89 (s, 3H), 3.52 (q, J=6.4 Hz, 2H), 2.94-2.78 (m, 2H), 2.78-2.56 (m, 4H), 1.07 (t, J=7.1 Hz, 6H). ¹³C NMR (101 MHz, DMSO) δ 166.87, 157.68, 153.25, 144.17, 141.22, 138.37, 131.10, 129.43, 128.83, 126.92, 124.54, 122.38, 117.13, 103.57, 55.41, 51.03, 46.68, 36.73, 11.13. C₂₃H₂₇N₃O₂, HRMS (ESI): m/z (M+H⁺): 378.2182 (calculated), 378.2170 (found).

### 6 fluoro-2 hydroxyquinoline-4-carboxylic Acid (17)

Yield: 42%. ¹H NMR (400 MHz, DMSO-d₆) δ 8.01 (dd, J=10.6, 2.9 Hz, 1H), 7.56-7.18 (m, 2H), 7.00 (s, 1H). ¹³C NMR (101 MHz, DMSO) δ 166.37, 160.77, 158.28, 155.92, 139.53, 139.50, 136.29, 125.63, 119.11, 118.87, 117.63, 117.54, 116.64, 116.54, 111.46, 111.21. C₁₀H₆FNO₃, EI-MS: m/z (M−H⁺): 206.2 (calculated), 206.0 (found).

### 2-chloro-N-[2-(dimethylamino)ethyl]-6-fluoroquinoline-4-carboxamide (18a)

Yield: 52%. ¹H NMR (400 MHz, DMSO-d₆) δ 8.85 (t, J=5.8 Hz, 1H), 8.10 (dd, J=9.2, 5.5 Hz, 1H), 8.03 (dd, J=10.2, 2.9 Hz, 1H), 7.81 (ddd, J=9.2, 8.2, 2.9 Hz, 1H), 7.69 (s, 1H), 3.46 (q, J=6.2 Hz, 2H), 2.55-2.43 (m, 2H), 2.25 (s, 6H). ¹³C NMR (101 MHz, DMSO) δ 164.63, 161.54, 159.09, 148.88, 148.85, 145.25, 145.19, 144.79, 131.24, 131.14, 124.39, 124.28, 121.31, 121.06, 120.91, 109.72, 109.48, 57.81, 45.06, 37.27. C₁₄H₁₅ClFN₃O, EI-MS: m/z (M+H⁺): 296.7 (calculated), 296.5 (found).

### 2-chloro-N-[2-(diethylamino)ethyl]-6-fluoroquinoline-4-carboxamide (18b)

Yield: 48%. ¹H NMR (400 MHz, DMSO-d₆) δ 8.81 (t, J=5.8 Hz, 1H), 8.06 (ddd, J=22.1, 9.7, 4.2 Hz, 2H), 7.79 (ddd, J=9.2, 8.3, 2.9 Hz, 1H), 7.67 (s, 1H), 3.41 (q, J=6.3 Hz, 2H), 2.65-2.51 (m, 6H), 0.99 (t, J=7.1 Hz, 6H). ¹³C NMR (101 MHz, DMSO) δ 164.60, 161.54, 159.08, 148.88, 148.86, 145.37, 145.31, 144.79, 131.23, 131.14, 124.36, 124.26, 121.33, 121.08, 120.86, 109.76, 109.52, 51.34, 46.40, 37.36, 11.64. C₁₆H₁₉ClFN₃O, EI-MS: m/z (M+H⁺): 324.8 (calculated), 324.4 (found).

### N-[2-(dimethylamino)ethyl]-6-fluoro-2-phenylquinoline-4-carboxamide (19a)

Yield: 81%. ¹H NMR (400 MHz, DMSO-d₆) δ 8.87 (t, J=5.7 Hz, 1H), 8.39-8.25 (m, 2H), 8.25-8.14 (m, 2H), 8.06 (dd, J=10.4, 2.9 Hz, 1H), 7.83-7.70 (m, 1H), 7.67-7.46 (m, 3H), 3.51 (q, =6.3 Hz, 2H), 2.60-2.49 (m, 2H), 2.28 (s, 6H). ¹³C NMR (101 MHz, DMSO) δ 166.17, 161.27, 158.83, 155.38, 155.35, 145.23, 142.51, 142.45, 137.98, 132.38, 132.29, 129.89, 128.89, 127.21, 124.24, 124.13, 120.32, 120.07, 117.47, 109.17, 108.94, 57.93, 45.10, 37.29. C₂₀H₂₀FN₃O, HRMS (ESI): m/z (M+H⁺): 338.1669 (calculated), 338.1661 (found).

### N-[2-(diethylamino)ethyl]-6-fluoro-2-phenylquinoline-4-carboxamide (19b)

Yield: 88%. ¹H NMR (400 MHz, DMSO-d₆) δ 8.94-8.78 (m, 1H), 8.39-8.25 (m, 2H), 8.25-8.13 (m, 2H), 8.07 (dd, J=10.4, 2.9 Hz, 1H), 7.74 (td, J=8.8, 2.9 Hz, 1H), 7.64-7.45 (m, 3H), 3.57-3.39 (m, 2H), 2.85-2.50 (m, 6H), 1.03 (t, J=7.1 Hz, 6H). ¹³C NMR (101 MHz, DMSO) δ 166.17, 161.27, 158.83, 155.39, 155.36, 145.25, 142.46, 137.98, 132.38, 132.29, 129.90, 128.89, 127.19, 124.23, 124.12, 120.34, 120.08, 117.49, 109.21, 108.98, 51.31, 46.53, 37.22, 11.53. C₂₂H₂₄FN₃O, HRMS (ESI): m/z (M+H⁺): 366.1982 (calculated), 366.1969 (found).

### Cell Lines and Viruses

Rhabdomyosarcoma (RD, ATCC, CCL-136), A172 (ATCC, CRL-1620), A549 (ATCC, CCL-185), HeLa (ATCC, CCL-2), and SH-SYSY (ATCC, CRL-2266) were maintained in a 37° C. in a 5% CO2 atmosphere. RD and A172 were cultured in Dulbecco's modified Eagle's medium (DMEM) with 10% fetal bovine serum (FBS) and 1% penicillin-streptomycin (P/S). SH-SYSY were cultured in 10% FBS and 1% P/S with 50% DMEM and 50% F-12 medium. All of the following EV-D68 strains used in this study were purchased from ATCC: US/KY/14-18953 (ATCC, NE-49132), US/MO/14-18947 (ATCC, NR-49129), US/MO/14-18949 (ATCC, NR-49130), US/IL/14-18952 (ATCC, NR-49131), US/IL/14-18956 (ATCC, NR-49133).

### Cytopathic Effect (CPE) Assay

For antiviral CPE assays, cells were grown to approximately 90% confluency (one day after seeding in a 96 well plate). For infection, growth media was removed and cells were washed with PBS supplemented with magnesium and calcium. Cells were infected with EV-D68 strains diluted in DMEM with 2% FBS and 30 mM MgCl2 and transferred to a 33° C. incubator in a 5% CO2 atmosphere (see, Smee, D. F.; et al., Antiviral Res. 2016, 131, 61-65). After 1 h, P/S was added to a final concentration of 1% followed by the addition of compounds. Plates were gently shaken on an orbital shaker for about 5 min before incubation. For RD, A172, and SH-SYSY cells, virus was added at a viral titer that resulted in complete CPE after 3 days of incubation (approximately an MOI of 0.01, 0.3, and 1 for RD, A172, and SH-SYSY, respectively). Media was aspirated and a 66 μg/ml solution of neutral red dye was used to stain viable cells in each well. Absorbance at 540 nm was measured using a Multiskan FC Microplate Photometer (ThermoFisher Scientific). The EC₅₀ values were calculated from best-fit dose response curves using GraphPad Prism, and all EC₅₀ values reported were done in triplicates.

### Cytotoxicity Assay

The cytotoxicity of each compound was determined using the neutral red cell viability assay. The assay was performed under similar conditions (incubation temperature, time, and media) as the CPE, but excluded viral infection. Data acquisition and analysis (CC₅₀) was performed similarly to the antiviral CPE assay, and all values are from triplicate experiments.

### Western Blotting

RD cells were infected with EV-D68 strain US/KY/14-18953 (MOI=1). Total proteins were extracted at 9 hpi using RAPI lysis buffer [50 mM Tris (pH 8.0), 1% NP-40, 0.1% SDS, 150 mM NaCl, 0.5% Sodium deoxycholate, 5 mM EDTA, 10 mM NaF, 10 mM NaPPi, 2 mM phenyl-methylsulfonyl, and 1 mM PMSF]. Equal amount of extracted total proteins were separated by electrophoresis and transferred to a polyvinylidene difluoride (PVDF) membrane. Viral protein VP1 or host GAPDH was recognized by rabbit anti-VP1 (GeneTex: GTX132313; 1:3,000 dilution) or mouse anti-GAPDH antibody (EMD Millipore: MAB374; 1:3,000 dilution), respectively, followed by detection using Horse radish peroxidase (HRP)-conjugated secondary antibody (ThermoFisher Scientific: 32430 or 656120; 1:3,000 dilutions) and Supersignal West Femto substrate (ThermoFisher Scientific).

### RNA Extraction and Quantitative Real-Time PCR (RT-qPCR).

RD cells were infected with EV-D68 strain US/KY/14-18953 (MOI=1). Total RNA was extracted at 9 hpi using Trizol reagents (ThermoFisher Scientific). After removing genomic DNA by RQ1 RNase-Free DNase (Promega), 1.2 μg of total RNA was used to synthesize first strand of cDNA of viral RNA and host mRNA using SuperScript III reverse transcriptase (ThermoFisher Scientific) and oligo (dT)₁₈. Viral RNA was amplified on a QuantStudio 5 Real-Time PCR System (ThermoFisher Scientific) using FastStart Universal SYBR Green Master (Rox) (Roche) and virus-specific primers: D68-F (5′-CGCTGAACTTGGCGTGGTCC-3′ (SEQ ID NO: 1)) and D68-R (5′-GGCTGCCCTGCTAAGAAAATTCTCC-3′ (SEQ ID NO: 2)). GAPDH (SEQ ID NO: 3) was amplified to serve as a control using GAPDH(SEQ ID NO: 3)-specific primers (GAPDH-F: 5′-ACACCCACTCCTCCACCTTTG-3′ (SEQ ID NO: 4) and GAPDH-R(SEQ ID NO: 3): 5′-CACCACCCTGTTGCTGTAGCC-3′ (SEQ ID NO: 5)). The amplification conditions were: 95° C. for 10 min; 40 cycles of 15 sec at 95° C. and 60 sec at 60° C. Melting curve analysis was performed to verify the specificity of each amplification.

### Immunostaining

RD cells were infected with EV-D68 strain US/KY/14-18953 (MOI=1). At 9 hpi, infected cells were fixed with 4% formaldehyde for 10 min followed by permeabilization with 0.2% Triton X-100 for another 10 min. After blocking with 10% bovine serum, cells were stained with rabbit anti-VP1 antibody (GeneTex: GTX132313) and followed by staining with anti-rabbit secondary antibody conjugated to Alexa-488 (ThermoFisher Scientific). Nucleus were stained with 300 nM DAPI (ThermoFisher Scientific) after secondary antibodies incubation. Fluorescent images were acquired using a Leica SP5-II spectral Confocal Microscope (Leica).

### Sodium Flux Assay

### Calcium Imaging in Acutely Dissociated Dorsal Root Ganglion (DRG) Neurons.

Dorsal root ganglion neurons were prepared as previously described²⁷ and were loaded for 30 minutes at 3TC with 3 μM Fura-2AM (Cat #F1221, Thermo Fisher, stock solution prepared at 1 mM in DMSO, 0.02% pluronic acid, Cat #P-3000MP, Life technologies) to follow changes in intracellular calcium ([Ca²⁺]_{c}) in a standard bath solution containing 139 mM NaCl, 3 mM KCl, 0.8 mM MgCl₂, 1.8 mM CaCl₂, 10 mM Na HEPES, pH 7.4, 5 mM glucose exactly as previously described²⁸ Fluorescence imaging was performed with an inverted microscope, NikonEclipseTi-U (Nikon Instruments Inc., Melville, N.Y.), using objective Nikon Fluor 4X and a Photometrics cooled CCD camera Cool SNAP ES² (Roper Scientific, Tucson, Ariz.) controlled by Nis Elements software (version 4.20, Nikon Instruments). The excitation light was delivered by a Lambda-LS system (Sutter Instruments, Novato, Calif.). The excitation filters (340±5 and 380±7) were controlled by a Lambda 10 to 2 optical filter change (Sutter Instruments). Fluorescence was recorded through a 505-nm dichroic mirror at 535±25 nm. To minimize photobleaching and phototoxicity, the images were taken every ˜10 seconds during the time-course of the experiment using the minimal exposure time that provided acceptable image quality. The changes in [Ca²⁺]_{c} were monitored by following a ratio of F₃₄₀/F₃₈₀, calculated after subtracting the background from both channels.

### Concentration Dependent Sodium-Channel Activated Calcium Imaging

DRG neurons were incubated overnight with testing compounds or Dibucaine at concentrations of 2 μM, 20 μM, and 50 μM as indicated. Following overnight incubation, cells were imaged via calcium imaging protocol with the following specifications: after a 1-minute baseline measurement, sodium channel agonist, Veratridine was added (30 μM) and indirect sodium influx was measured by obtained fluorescence ratio of 340 nm/380 nm of resulting sodium-channel triggered calcium influx. Total data collection time for each coverslip was 7 minutes in length.

### Example II

This example describes the further development of broad-spectrum enterovirus antivirals based on quinoline scaffold.

### Profiling the Broad-Spectrum Antiviral Activity of Known 2C Inhibitors Against Non-Polio Enteroviruses

The viral 2C protein is relatively conserved among non-polio enteroviruses. Therefore, experiments were conducted that hypothesized that 2C inhibitors will have broad-spectrum antiviral activity against non-polio enteroviruses. To test this hypothesis, experiments were conducted that first started by profiling the broad-spectrum antiviral activity of literature reported 2C inhibitors (see, R. Ulferts, et al., Antimicrob. Agents. Chemother. 60 (2016) 2627-2638; E. Rhoden, et al., Antimicrob. Agents. Chemther. 59 (2015) 7779-7781; L. Sun, et al., Antiviral Res. 131 (2016) 61-65; L. Bauer, et al., ACS Infect. Dis. 5 (2019) 1609-1623) against the non-polio enteroviruses including EV-D68, EV-A71 and CVB3 in cell culture using cytopathic effect (CPE) assay. It was found that dibucaine, fluoxetine, pirlindole mesylate, and formoterol all inhibited EV-D68 and CVB3, but not the EV-A71 virus. Moreover, they all had a low selectivity index (SI₅₀<50). The only exception was guanidine, which showed broad-spectrum antiviral activity against all three viruses, but only at very high concentrations (200 μM-900 μM). Overall, the existing 2C inhibitors appears to lack the broad-spectrum antiviral activity against non-polio enteroviruses. Nevertheless, the results of guanidine suggested that it might be feasible to develop broad-spectrum antivirals against non-polio enteroviruses. As such, additional experiments were conducted to develop viral 2C inhibitors as broad-spectrum antivirals against not only EV-D68, but also EV-A71 and CVB3.

### Synthesis of Quinoline Analogs

Synthesis of quinoline analogs started with Suzuki-Miyaura cross-coupling of 2-chloroquinoline-4-carboxylic acid 1 with various boronic acids 2 (Scheme 2). The carboxylic acid intermediate 3 was then reacted with various diamines to give the amide intermediate 5. Deprotection of Boc by TFA gave the final product 6. Overall, this is a highly efficient synthesis with an overall yield of 53.2 to 76.9%.

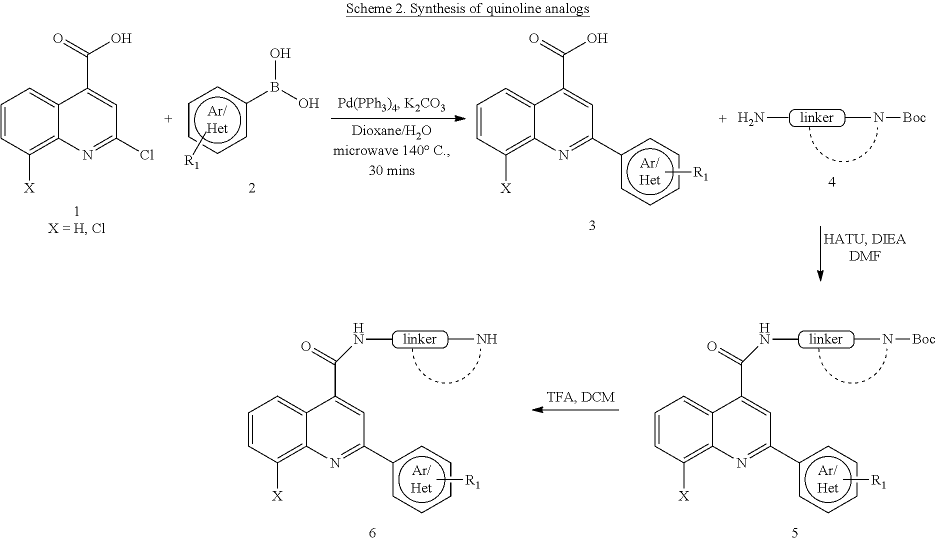

Selection of Lead Compounds with Potent Antiviral Activity (EC₅₀<1 μM) and a High Selectivity Index (SI₅₀>100) Against EV-D68 US/KY/14-18953 Virus

The purpose of the current structure-activity relationship studies was to provide additional lead compounds with potent antiviral activity (EC₅₀<1 μM) and a high selectivity index (SI₅₀>100) for the following broad-spectrum antiviral activity profiling and mouse microsomal stability test. It is expected that majority of the compounds will be filtered during this multi-parameter optimization process; therefore it is imperative to obtain as many backup molecules as possible in order to increase the chance of success.

The antiviral activity of all compounds was initially tested against EV-D68 US/KY/14-18953 virus in RD cells using the CPE assay. The cytotoxicity assay was performed in parallel in RD cells using the neutral red method. Next, compounds with potent antiviral activity (EC₅₀<1 μM) and a high selectivity index (SI₅₀>100) against EV-D68 US/KY/14-18953 were selected for further testing against additional EV-D68, EV-A71 and CVB3 viruses. In parallel, promising lead compounds were profiled for mouse microsomal stability. Overall, this is a multiparameter optimization approach with an aim of identifying lead compounds with broad-spectrum antiviral activity and favorable in vitro pharmacokinetic properties.

Compounds 6aa and 6bf were identified as potent EV-D68 antivirals from the previous round of structure-activity relationship studies of dibucaine (see, R. Musharrafieh, et al., J. Med. Chem. 62 (2019) 4074-4090), and both compounds were included as references (Table 9). For the series of compounds with 1-position substitution being benzene (6aa-6aq), it was found that replacing the 4-position amide linkage with ester abolished the antiviral activity (compound 6ab vs 6aa). Extending the linker between amide to terminal amine from ethyl to propyl also reduced the antiviral activity (6aa vs 6ac and 6ad). Compound 6ae with a terminal imidazole group was less active than compound 6aa that had a dimethyl amine group. Compounds 6ag, 6ah, 6ai, 6aj, 6ak, 6al, and 6 am all had a single-digit micromolar EC₅₀ value, but their low selectivity indexes (SI=3.2-16.1) did not warrant further investigation of these compounds. Compounds 6af, 6an, and 6ap had similar antiviral activity as compound 6aa, but their selectivity indexes did not pass the selectivity threshold (SI>100). The only compound met the criteria was compound 6aq (EC₅₀=0.5±0.3 μM; SI₅₀=131.6). It is known that benzene substituent might be easily metabolized, especially through 4-hydroxylation. We therefore made an effort to block this metabolism by introducing either deuterium (6ar-6au) or fluorine (6av-6be). For compounds with deuterated benzene at 1-position (6ar-6au), compound 6ar was the most potent and selective candidate (EC₅₀=0.3±0.2 μM; SI₅₀=268.7), and it was selected for further characterization. For compounds with fluorine-substituted benzene at 1-position (6av-6be), the four compounds 6av, 6aw, 6ax, and 6ba met the selection criteria (EC₅₀<1 μM; SI₅₀>100). For compounds containing thiophene at 1-position (6bf-6bn), the five compounds 6bf, 6bg, 6bi, 6bj, and 6b1 had the desired potency and selectivity index for further development. For compounds with furan at 1-position (6bo-6bs), only compound 6bo met the selection criteria.

TABLE 9
Antiviral activity against EV-D68 US/KY/14-18953.

| | |
|---|---|
| 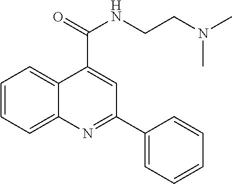 [SMILES: CN(C)CCNC(=O)c1cc(-c2ccccc2)nc2ccccc12] | 6aa |
| EC₅₀ = 0.4 ± 0.2 μM | |
| CC₅₀ = 73.7 ± 19.1 μM | |
| SI₅₀ = 184.3 | |
| 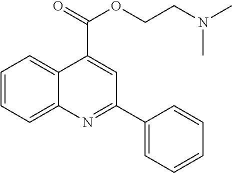 [SMILES: CN(C)CCOC(=O)c1cc(-c2ccccc2)nc2ccccc12] | 6ab |
| EC₅₀ >50.0 μM | |
| CC₅₀ = 78.9 μM | |
| SI₅₀ = N.A. | |
| 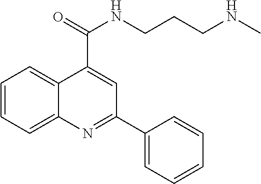 [SMILES: CNCCCNC(=O)c1cc(-c2ccccc2)nc2ccccc12] | 6ac |
| EC₅₀ = 8.9 ± 6.4 μM | |
| CC₅₀ = 93.6 ± 22.0 μM | |
| SI₅₀ = 10.5 | |
| 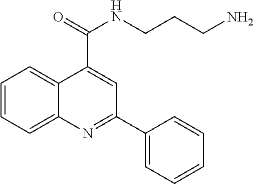 [SMILES: NCCCNC(=O)c1cc(-c2ccccc2)nc2ccccc12] | 6ad |
| EC₅₀ = 7.2 ± 1.2 μM | |
| CC₅₀ = 168.3 ± 27.2 μM | |
| SI₅₀ = 23.4 | |
| 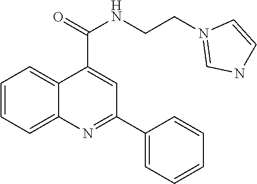 | 6ae |
| EC₅₀ = 11.9 ± 2.5 μM | |
| CC₅₀ = 134.3 ± 37.9 μM | |
| SI₅₀ = 11.3 | |
| 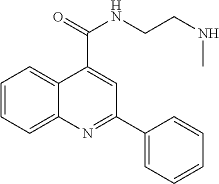 [SMILES: CNCCNC(=O)c1cc(-c2ccccc2)nc2ccccc12] | 6af |
| EC₅₀ = 0.9 ± 0.3 μM | |
| CC₅₀ = 47.8 ± 3.6 μM | |
| SI₅₀ = 53.1 | |
| 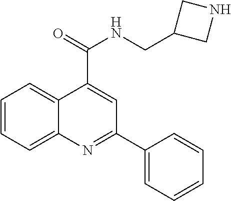 [SMILES: O=C(NCC1CNC1)c1cc(-c2ccccc2)nc2ccccc12] | 6ag |
| EC₅₀ = 5.8 ± 0.3 μM | |
| CC₅₀ = 43.2 ± 4.5 μM | |
| SI₅₀ = 7.4 | |
| 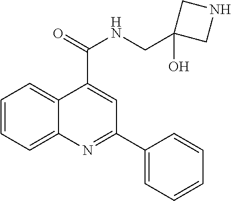 [SMILES: O=C(NCC1(O)CNC1)c1cc(-c2ccccc2)nc2ccccc12] | 6ah |
| EC₅₀ = 5.1 ± 0.4 μM | |
| CC₅₀ = 82.2 ± 19.9 μM | |
| SI₅₀ = 16.1 | |
| 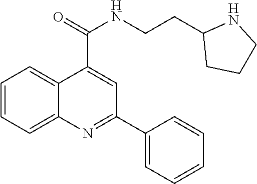 [SMILES: O=C(NCCC1CCCN1)c1cc(-c2ccccc2)nc2ccccc12] | 6ai |
| EC₅₀ = 6.0 ± 0.3 μM | |
| CC₅₀ = 19.1 ± 3.7 μM | |
| SI₅₀ = 3.2 | |
| 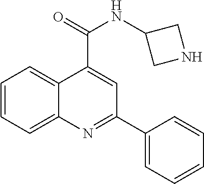 [SMILES: O=C(NC1CNC1)c1cc(-c2ccccc2)nc2ccccc12] | 6aj |
| EC₅₀ = 4.2 ± 0.2 μM | |
| CC₅₀ = 36.6 ± 11.2 μM | |
| SI₅₀ = 8.7 | |
| 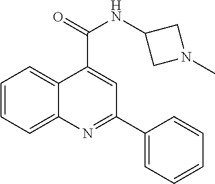 [SMILES: CN1CC(NC(=O)c2cc(-c3ccccc3)nc3ccccc23)C1] | 6ak |
| EC₅₀ = 2.9 ± 1.2 μM | |
| CC₅₀ = 46.8 ± 13.6 μM | |
| SI₅₀ = 16.1 | |
| 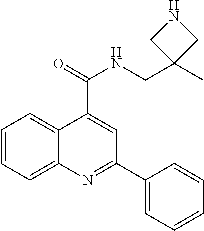 [SMILES: CC1(CNC(=O)c2cc(-c3ccccc3)nc3ccccc23)CNC1] | 6al |
| EC₅₀ = 6.9 ± 1.3 μM | |
| CC₅₀ = 49.5 ± 13.7 μM | |
| SI₅₀ = 7.2 | |
| 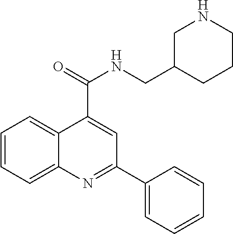 [SMILES: O=C(NCC1CCCNC1)c1cc(-c2ccccc2)nc2ccccc12] | 6am |
| EC₅₀ = 7.1 ± 2.5 μM | |
| CC₅₀ = 30.1 ± 7.2 μM | |
| SI₅₀ = 4.2 | |
| 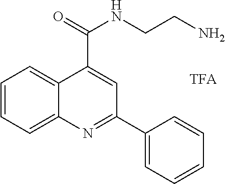 [SMILES: NCCNC(=O)c1cc(-c2ccccc2)nc2ccccc12] | 6an |
| EC₅₀ = 1.2 ± 0.4 μM | |
| CC₅₀ = 44.9 ± 8.2 μM | |
| SI₅₀ = 37.4 | |
| 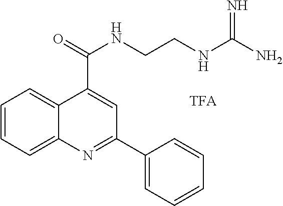 [SMILES: N=C(N)NCCNC(=O)c1cc(-c2ccccc2)nc2ccccc12] | 6ao |
| EC₅₀ = 3.3 ± 1.1 μM | |
| CC₅₀ >200.0 μM | |
| SI₅₀ >60.6 | |
| 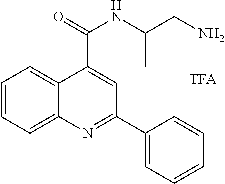 [SMILES: CC(CN)NC(=O)c1cc(-c2ccccc2)nc2ccccc12] | 6ap |
| EC₅₀ = 1.2 ± 0.3 μM | |
| CC₅₀ = 89.1 ± 3.2 μM | |
| SI₅₀ = 74.3 | |
| 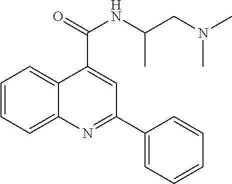 [SMILES: CC(CN(C)C)NC(=O)c1cc(-c2ccccc2)nc2ccccc12] | 6aq |
| EC₅₀ = 0.5 ± 0.3 μM | |
| CC₅₀ = 65.8 ± 16.8 μM | |
| SI₅₀ = 131.6 | |
| 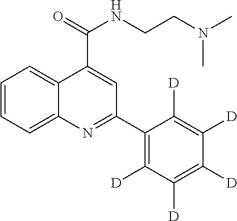 [SMILES: [2H]c1c([2H])c([2H])c(-c2cc(C(=O)NCCN(C)C)c3ccccc3n2)c([2H])c1[2H]] | 6ar |
| EC₅₀ = 0.3 ± 0.2 μM | |
| CC₅₀ = 80.6 ± 3.3 μM | |
| SI₅₀ = 268.7 | |
| 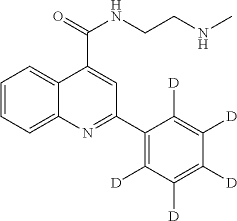 [SMILES: [2H]c1c([2H])c([2H])c(-c2cc(C(=O)NCCNC)c3ccccc3n2)c([2H])c1[2H]] | 6as |
| EC₅₀ = 1.1 ± 0.3 μM | |
| CC₅₀ = 100.8 ± 3.2 μM | |
| SI₅₀ = 91.6 | |
| 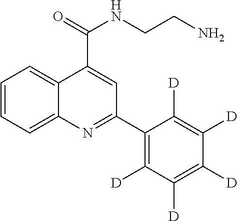 [SMILES: [2H]c1c([2H])c([2H])c(-c2cc(C(=O)NCCN)c3ccccc3n2)c([2H])c1[2H]] | 6at |
| EC₅₀ = 2.2 ± 0.2 μM | |
| CC₅₀ = 100.3 ± 12.1 μM | |
| SI₅₀ = 45.6 | |
| 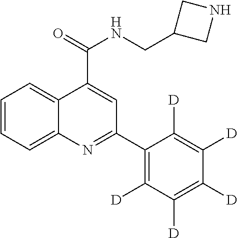 [SMILES: [2H]c1c([2H])c([2H])c(-c2cc(C(=O)NCC3CNC3)c3ccccc3n2)c([2H])c1[2H]] | 6au |
| EC₅₀ = 6.4 ± 0.2 μM | |
| CC₅₀ = 188.5 ± 20.4 μM | |
| SI₅₀ = 29.5 | |
| 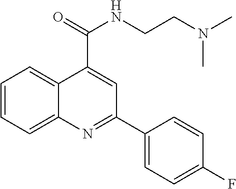 [SMILES: CN(C)CCNC(=O)c1cc(-c2ccc(F)cc2)nc2ccccc12] | 6av |
| EC₅₀ = 0.1 ± 0.1 μM | |
| CC₅₀ = 36.2 ± 3.1 μM | |
| SI₅₀ = 362.0 | |
| 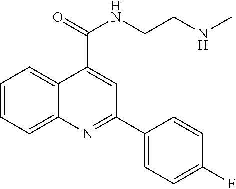 [SMILES: CNCCNC(=O)c1cc(-c2ccc(F)cc2)nc2ccccc12] | 6aw |
| EC₅₀ = 0.2 ± 0.1 μM | |
| CC₅₀ = 32.4 ± 4.1 μM | |
| SI₅₀ = 162.0 | |
| 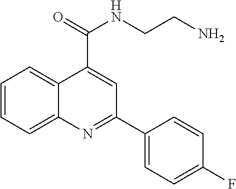 [SMILES: NCCNC(=O)c1cc(-c2ccc(F)cc2)nc2ccccc12] | 6ax |
| EC₅₀ = 0.4 ± 0.1 μM | |
| CC₅₀ = 48.2 ± 2.3 μM | |
| SI₅₀ = 120.5 | |
| 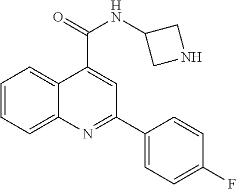 [SMILES: O=C(NC1CNC1)c1cc(-c2ccc(F)cc2)nc2ccccc12] | 6ay |
| EC₅₀ = 1.1 ± 0.1 μM | |
| CC₅₀ = 46.7 ± 3.7 μM | |
| SI₅₀ = 42.5 | |
| 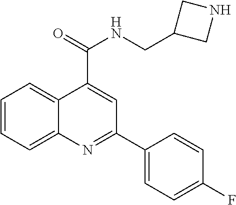 [SMILES: O=C(NCC1CNC1)c1cc(-c2ccc(F)cc2)nc2ccccc12] | 6az |
| EC₅₀ = 1.2 ± 0.2 μM | |
| CC₅₀ = 50.6 ± 3.1 μM | |
| SI₅₀ = 42.2 | |
| 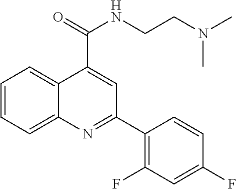 [SMILES: CN(C)CCNC(=O)c1cc(-c2ccc(F)cc2F)nc2ccccc12] | 6ba |
| EC₅₀ = 0.3 ± 0.2 μM | |
| CC₅₀ = 36.7 ± 5.6 μM | |
| SI₅₀ = 122.3 | |
| 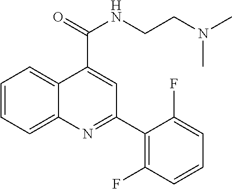 [SMILES: CN(C)CCNC(=O)c1cc(-c2c(F)cccc2F)nc2ccccc12] | 6bb |
| EC₅₀ = 12.1 ± 2.5 μM | |
| CC₅₀ = 93.0 ± 4.1 μM | |
| SI₅₀ = 7.7 | |
| 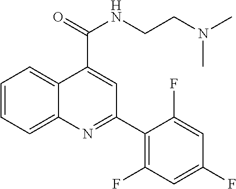 [SMILES: CN(C)CCNC(=O)c1cc(-c2c(F)cc(F)cc2F)nc2ccccc12] | 6bc |
| EC₅₀ = 3.6 ± 1.2 μM | |
| CC₅₀ = 39.2 ± 25.4 μM | |
| SI₅₀ = 10.9 | |
| 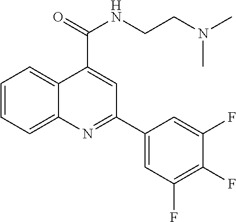 [SMILES: CN(C)CCNC(=O)c1cc(-c2cc(F)c(F)c(F)c2)nc2ccccc12] | 6bd |
| EC₅₀ >300 μM | |
| CC₅₀ = 38.0 ± 5.2 μM | |
| SI₅₀ = N.A. | |
| 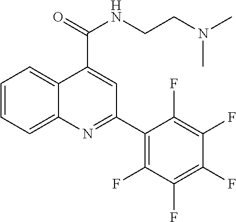 [SMILES: CN(C)CCNC(=O)c1cc(-c2c(F)c(F)c(F)c(F)c2F)nc2ccccc12] | 6be |
| EC₅₀ = 10.8 ± 2.5 μM | |
| CC₅₀ = 147.8 ± 14.3 μM | |
| SI₅₀ = 13.7 | |
| 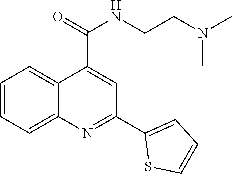 [SMILES: CN(C)CCNC(=O)c1cc(-c2cccs2)nc2ccccc12] | 6bf |
| EC₅₀ = 0.04 ± 0.02 μM | |
| CC₅₀ = 42.3 ± 8.3 μM | |
| SI₅₀ = 1057.5 | |
| 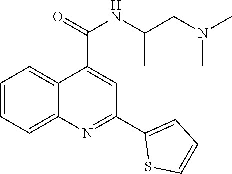 [SMILES: CC(CN(C)C)NC(=O)c1cc(-c2cccs2)nc2ccccc12] | 6bg |
| EC₅₀ = 0.1 ± 0.1 μM | |
| CC₅₀ = 20.6 ± 0.9 μM | |
| SI₅₀ = 206.0 | |
| 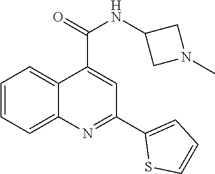 [SMILES: CN1CC(NC(=O)c2cc(-c3cccs3)nc3ccccc23)C1] | 6bh |
| EC₅₀ = 1.0 ± 0.3 μM | |
| CC₅₀ = 42.6 ± 4.7 μM | |
| SI₅₀ = 42.6 | |
| 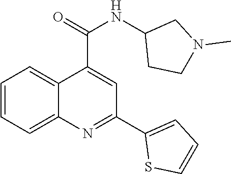 [SMILES: CN1CCC(NC(=O)c2cc(-c3cccs3)nc3ccccc23)C1] | 6bi |
| EC₅₀ = 0.1 ± 0.2 μM | |
| CC₅₀ = 39.0 ± 3.4 μM | |
| SI₅₀ = 390.0 | |
| 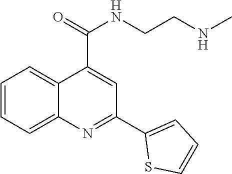 [SMILES: CNCCNC(=O)c1cc(-c2cccs2)nc2ccccc12] | 6bj |
| EC₅₀ = 0.3 ± 0.1 μM | |
| CC₅₀ = 103.1 ± 49.5 μM | |
| SI₅₀ = 343.7 | |
| 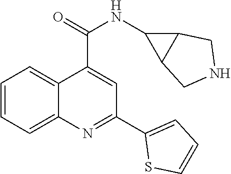 [SMILES: O=C(NC1C2CNCC21)c1cc(-c2cccs2)nc2ccccc12] | 6bk |
| EC₅₀ = 6.4 ± 2.2 μM | |
| CC₅₀ = 34.5 ± 6.8 μM | |
| SI₅₀ = 5.4 | |
| 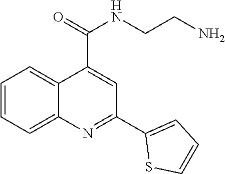 [SMILES: NCCNC(=O)c1cc(-c2cccs2)nc2ccccc12] | 6bl |
| EC₅₀ = 0.4 ± 0.1 μM | |
| CC₅₀ = 42.1 ± 13.4 μM | |
| SI₅₀ = 105.3 | |
| 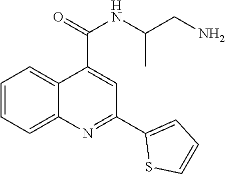 [SMILES: CC(CN)NC(=O)c1cc(-c2cccs2)nc2ccccc12] | 6bm |
| EC₅₀ = 0.5 ± 0.2 μM | |
| CC₅₀ = 23.8 ± 2.6 μM | |
| SI₅₀ = 47.6 | |
| 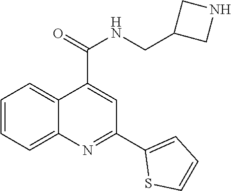 [SMILES: O=C(NCC1CNC1)c1cc(-c2cccs2)nc2ccccc12] | 6bn |
| EC₅₀ = 1.2 ± 0.3 μM | |
| CC₅₀ = 28.3 ± 7.6 μM | |
| SI₅₀ = 23.6 | |
| 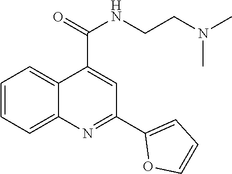 [SMILES: CN(C)CCNC(=O)c1cc(-c2ccco2)nc2ccccc12] | 6bo |
| EC₅₀ = 1.1 ± 0.4 μM | |
| CC₅₀ = 198.6 ± 9.4 μM | |
| SI₅₀ = 180.5 | |
| 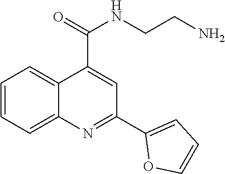 [SMILES: NCCNC(=O)c1cc(-c2ccco2)nc2ccccc12] | 6bp |
| EC₅₀ = 4.4 ± 0.8 μM | |
| CC₅₀ = 146.9 ± 12.5 μM | |
| SI₅₀ = 33.4 | |
| 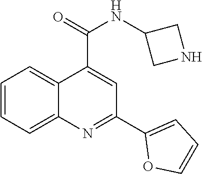 [SMILES: O=C(NC1CNC1)c1cc(-c2ccco2)nc2ccccc12] | 6bq |
| EC₅₀ = 11.1 ± 3.2 μM | |
| CC₅₀ = 160.6 ± 4.5 μM | |
| SI₅₀ = 14.5 | |
| 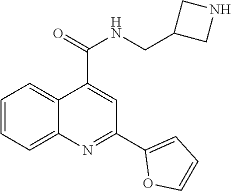 [SMILES: O=C(NCC1CNC1)c1cc(-c2ccco2)nc2ccccc12] | 6br |
| EC₅₀ = 11.5 ± 2.2 μM | |
| CC₅₀ = 170.3 ± 16.1 μM | |
| SI₅₀ = 14.8 | |
| 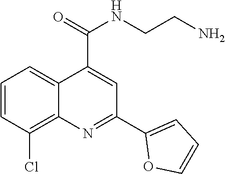 [SMILES: NCCNC(=O)c1cc(-c2ccco2)nc2c(Cl)cccc12] | 6bs |
| EC₅₀ = 1.0 ± 0.1 μM | |
| CC₅₀ = 50.5 ± 4.9 μM | |
| SI₅₀ = 50.5 | |

Antiviral efficacy was determined in the CPE assay. Cytotoxicity was determined using the neutral red method. The results are the mean ± standard deviation of three repeats.
SI = selectivity index (CC₅₀/EC₅₀).
N.A. = not applicable.

Overall, in addition to the two reference compounds (6aa and 6bf), 11 additional candidates (6aq, 6ar, 6av, 6aw, 6ax, 6ba, 6bg, 6bi, 6bj, 6b1, and 6bo) were identified with high antiviral potency (EC₅₀<1 μM) and an optimal selectivity index (SI₅₀>100) that warrant further profiling of broad-spectrum antiviral activity and mouse microsomal stability.

Broad-Spectrum Antiviral Activity of Lead Compounds Against EV-D68, EV-A71 and CVB3, and their Mouse Microsomal Stability

Lead candidates with potent antiviral activity (EC₅₀<1 μM) and a high selectivity index (SI₅₀>100) against EV-D68 US/KY/14-18953 virus from Table 9 were selected for further testing against other enteroviruses, including EV-D68 US/MO/14-18947, EV-A71 Tainan/4643/98, EV-A71 US/AK/16-19516, and CVB3 viruses. In total 13 compounds met these criteria. In parallel, promising lead compounds were further tested for mouse microsomal stability. It was found that all 13 compounds had potent antiviral activity against both EV-D68 strains (US/KY and US/MO) with EC₅₀ values ranging from single-digit to submicromolar. Encouragingly, all 13 compounds also had potent antiviral activity against CVB3 virus, and the EC₅₀ values were very similar to that of EV-D68 viruses. When tested against the two EV-A71 strains, Tainan and US/AK, most of the compounds (6aa, 6ar, 6av, 6aw, 6ax, 6ba, 6bf, 6bg, 6bi) showed potent inhibition as well with low single-digit micromolar EC₅₀ values. The four compounds 6aq, 6bj, 6b1 and 6bo were less potent and had high single-digit to double-digit micromolar EC₅₀ values. Overall, the pattern of broad-spectrum antiviral activity for compounds shown in Table 10 matches with that of known 2C inhibitors shown in Table 11: the 2C inhibitors have potent antiviral activity against EV-D68 and CVB3, but they are generally less active against EV-A71. Nevertheless, compounds 6ar, 6av, 6aw, 6bg, 6bi all had single-digit micromolar EC₅₀ values against EV-A71, in addition to their potent antiviral activity against EV-D68 and CVB3 viruses.

TABLE 10
Broad-spectrum antiviral activity against EV-D68, EV-A71, CVB3, and mouse microsomal stability.

| | | | | Mouse | |
|---|---|---|---|---|---|
| | | | Anti-CVB3 | microsomal | |
| | Anti-EV-D68 | Anti-EV-A71 | in Vero C1008 cells | stability T_{1/2} | CL_{Int(liver)} |
| Structure | in RD cells (μM) | in RD cells (μM) | (μM) | (mins) | (ml/min/kg) |
| 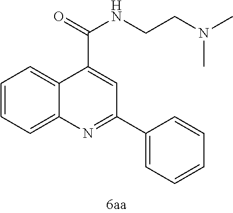 [SMILES: CN(C)CCNC(=O)c1cc(-c2ccccc2)nc2ccccc12] | US/KY/14-18953 EC₅₀ = 0.4 ± 0.2 CC₅₀ = 73.7 ± 19.1 SI₅₀ = 184.3 US/MO/14-18947 EC₅₀ = 0.2 ± 0.07 CC₅₀ = 73.7 ± 19.1 SI₅₀ = 368.5 | Tainan EC₅₀ = 4.2 ± 0.5 CC₅₀ = 73.7 ± 19.1 SI₅₀ = 17.5 US/AK EC₅₀ >10 | EC₅₀ = 0.3 ± 0.2 CC₅₀ = 116.2 ± 56.3 SI₅₀ = 387.3 | 22.4 | 245.5 |
| 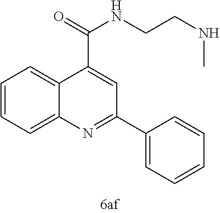 [SMILES: CNCCNC(=O)c1cc(-c2ccccc2)nc2ccccc12] | US/KY/14-18953 EC₅₀ = 0.9 ± 0.3 CC₅₀ = 47.8 ± 3.6 SI₅₀ = 53.1 US/MO/14-18947 EC₅₀ = 0.6 ± 0.3 CC₅₀ = 47.8 ± 3.6 SI₅₀ = 79.7 | Tainan EC₅₀ = 7.8 CC₅₀ = 47.8 ± 3.6 SI₅₀ = 6.1 US/AK EC₅₀ >10 | EC₅₀ = 1.1 ± 0.2 CC₅₀ >30.0 SI₅₀ >27.3 | 60.2 | 91.2 |
| 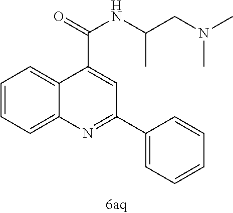 [SMILES: CC(CN(C)C)NC(=O)c1cc(-c2ccccc2)nc2ccccc12] | US/KY/14-18953 EC₅₀ = 0.5 ± 0.3 CC₅₀ = 65.8 ± 16.8 SI₅₀ = 131.6 US/MO/14-18947 EC₅₀ = 0.3 ± 0.1 CC₅₀ = 65.8 ± 16.8 SI₅₀ = 219.3 | Tainan EC₅₀ = 18.3 ± 4.2 CC₅₀ = 65.8 ± 16.8 SI₅₀ = 3.6 US/AK EC₅₀ = 11.7 ± 3.0 CC₅₀ = 65.8 ± 16.8 SI₅₀ = 5.6 | EC₅₀ = 2.0 ± 0.2 CC₅₀ = 185.9 ± 13.9 SI₅₀ = 93.0 | N.T. | N.T. |
| 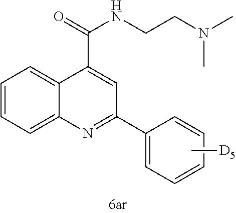 | US/KY/14-18953 EC₅₀ = 0.3 ± 0.2 CC₅₀ = 80.6 ± 3.3 SI₅₀ = 268.7 US/MO/14-18947 EC₅₀ = 0.2 ± 0.1 CC₅₀ = 80.6 ± 3.3 SI₅₀ = 403.0 | Tainan EC₅₀ = 5.2 ± 0.2 CC₅₀ = 80.6 ± 3.3 SI₅₀ = 15.5 US/AK EC₅₀ = 4.9 ± 0.2 CC₅₀ = 80.6 ± 3.3 SI₅₀ = 16.4 | EC₅₀ = 0.4 ± 0.1 CC₅₀ = 128.8 ± 25.8 SI₅₀ = 322.0 | 23.6 | 232.2 |
| 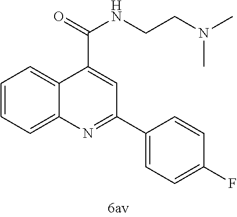 [SMILES: CN(C)CCNC(=O)c1cc(-c2ccc(F)cc2)nc2ccccc12] | US/KY/14-18953 EC₅₀ = 0.1 ± 0.1 CC₅₀ = 36.2 ± 3.1 SI₅₀ = 362.0 US/MO/14-18947 EC₅₀ = 0.05 ± 0.02 CC₅₀ = 36.2 ± 3.1 SI₅₀ = 724.0 | Tainan EC₅₀ = 2.2 ± 0.1 CC₅₀ = 36.2 ± 3.1 SI₅₀ = 16.5 US/AK EC₅₀ = 2.3 ± 0.1 CC₅₀ = 36.2 ± 3.1 SI₅₀ = 15.7 | EC₅₀ = 0.06 ± 0.04 CC₅₀ = 48.8 ± 17.7 SI₅₀ = 813.3 | 34.2 | 160.5 |
| 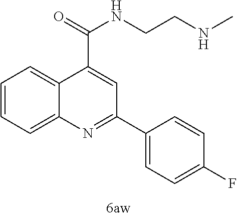 [SMILES: CNCCNC(=O)c1cc(-c2ccc(F)cc2)nc2ccccc12] | US/KY/14-18953 EC₅₀ = 0.2 ± 0.1 CC₅₀ = 32.4 ± 4.1 SI₅₀ = 162 US/MO/14-18947 EC₅₀ = 0.1 ± 0.02 CC₅₀ = 32.4 ± 4.1 SI₅₀ = 324.0 | Tainan EC₅₀ = 3.1 ± 1.0 CC₅₀ = 32.4 ± 4.1 SI₅₀ = 10.5 US/AK EC₅₀ = 3.6 ± 0.2 CC₅₀ = 32.4 ± 4.1 SI₅₀ = 9.0 | EC₅₀ = 0.2 ± 0.2 CC₅₀ = 40.1 ± 13.2 SI₅₀ = 200.5 | 114.7 | 47.9 |
| 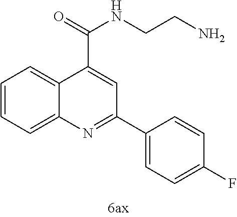 [SMILES: NCCNC(=O)c1cc(-c2ccc(F)cc2)nc2ccccc12] | US/KY/14-18953 EC₅₀ = 0.4 ± 0.1 CC₅₀ = 48.2 ± 2.3 SI₅₀ = 120.5 US/MO/14-18947 EC₅₀ = 0.3 ± 0.2 CC₅₀ = 48.2 ± 2.3 SI₅₀ = 160.7 | Tainan EC₅₀ = 9.0 ± 0.3 CC₅₀ = 48.2 ± 2.3 SI₅₀ = 5.4 US/AK EC₅₀ = 14.0 ± 2.1 CC₅₀ = 48.2 ± 2.3 SI₅₀ = 3.4 | EC₅₀ = 0.4 ± 0.1 CC₅₀ = 52.3 ± 5.0 SI₅₀ = 130.8 | 114.1 | 48.1 |
| 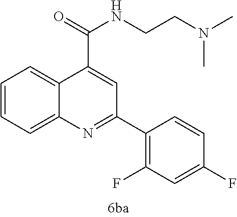 [SMILES: CN(C)CCNC(=O)c1cc(-c2ccc(F)cc2F)nc2ccccc12] | US/KY/14-18953 EC₅₀ = 0.3 ± 0.2 CC₅₀ = 36.7 ± 5.6 SI₅₀ = 122.3 US/MO/14-18947 EC₅₀ = 0.2 ± 0.1 CC₅₀ = 36.7 ± 5.6 SI₅₀ = 183.5 | Tainan EC₅₀ = 7.9 ± 0.5 CC₅₀ = 36.7 ± 5.6 SI₅₀ = 4.6 US/AK EC₅₀ >10 | EC₅₀ = 0.4 ± 0.2 CC₅₀ = 92.3 ± 3.2 SI₅₀ = 230.8 | N.T. | N.T. |
| 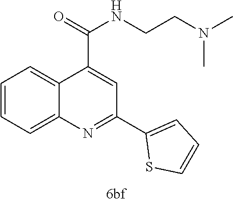 [SMILES: CN(C)CCNC(=O)c1cc(-c2cccs2)nc2ccccc12] | US/KY/14-18953 EC₅₀ = 0.04 ± 0.02 CC₅₀ = 42.3 ± 8.3 SI₅₀ = 1057.5 US/MO/14-18947 EC₅₀ = 0.05 ± 0.03 CC₅₀ = 42.3 ± 8.3 SI₅₀ = 846.0 | Tainan EC₅₀ = 2.9 ± 0.2 CC₅₀ = 42.3 ± 8.3 SI₅₀ = 14.6 US/AK EC₅₀ >10 | EC₅₀ = 0.07 ± 0.0 μM CC₅₀ = 94.5 ± 4.1 SI₅₀ = 1350.0 | 26.6 | 206.6 |
| 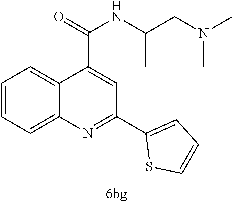 [SMILES: CC(CN(C)C)NC(=O)c1cc(-c2cccs2)nc2ccccc12] | US/KY/14-18953 EC₅₀ = 0.1 ± 0.1 CC₅₀ = 20.6 ± 0.9 SI₅₀ = 206.0 US/MO/14-18947 EC₅₀ = 0.1 ± 0.03 CC₅₀ = 20.6 ± 0.9 SI₅₀ = 206.0 | Tainan EC₅₀ = 7.9 ± 0.4 CC₅₀ = 20.6 ± 0.9 SI₅₀ = 2.6 US/AK EC₅₀ = 8.8 ± 0.8 CC₅₀ = 20.6 ± 0.9 SI₅₀ = 2.3 | EC₅₀ = 0.4 ± 0.2 CC₅₀ = 111.8 ± 7.6 SI₅₀ = 279.5 | N.T. | N.T. |
| 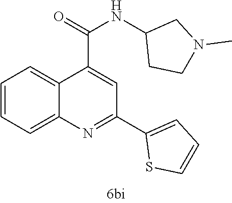 [SMILES: CN1CCC(NC(=O)c2cc(-c3cccs3)nc3ccccc23)C1] | US/KY/14-18953 EC₅₀ = 0.1 ± 0.2 CC₅₀ = 39.0 ± 3.4 SI₅₀ = 390.0 US/MO/14-18947 EC₅₀ = 0.06 ± 0.03 CC₅₀ = 39.0 ± 3.4 SI₅₀ = 650.0 | Tainan EC₅₀ = 6.3 ± 0.6 CC₅₀ = 39.0 ± 3.4 SI₅₀ = 6.2 US/AK EC₅₀ = 8.0 ± 0.5 CC₅₀ = 39.0 ± 3.4 SI₅₀ = 4.9 | EC₅₀ = 0.2 ± 0.1 CC₅₀ = 90.3 ± 5.9 SI₅₀ = 451.5 | N.T. | N.T. |
| 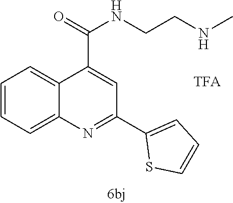 [SMILES: CNCCNC(=O)c1cc(-c2cccs2)nc2ccccc12] | US/KY/14-18953 EC₅₀ = 0.3 ± 0.1 CC₅₀ = 103.1 ± 49.5 SI₅₀ = 343.7 US/MO/14-18947 EC₅₀ = 0.2 ± 0.1 CC₅₀ = 103.1 ± 49.5 SI₅₀ = 515.5 | Tainan EC₅₀ = 8.4 ± 2.6 CC₅₀ = 103.1 ± 49.5 SI₅₀ = 12.3 US/AK EC₅₀ = 11.5 ± 0.8 CC₅₀ = 103.1 ± 49.5 SI₅₀ = 9.0 | EC₅₀ = 0.3 ± 0.05 CC₅₀ = 70.7 ± 4.8 SI₅₀ = 235.7 | 75.4 | 72.8 |
| 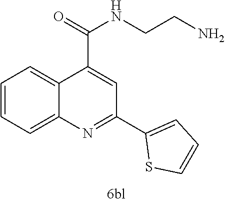 [SMILES: NCCNC(=O)c1cc(-c2cccs2)nc2ccccc12] | US/KY/14-18953 EC₅₀ = 0.4 ± 0.1 CC₅₀ = 42.1 ± 13.4 SI₅₀ = 105.3 US/MO/14-18947 EC₅₀ = 0.2 ± 0.1 CC₅₀ = 42.1 ± 13.4 SI₅₀ = 210.5 | Tainan EC₅₀ = 38.3 ± 6.5 CC₅₀ = 42.1 ± 13.4 SI₅₀ = 1.1 US/AK EC₅₀ >30.0 | EC₅₀ = 0.6 ± 0.2 CC₅₀ = 63.7 ± 3.2 SI₅₀ = 106.2 | 100.8 | 54.5 |
| 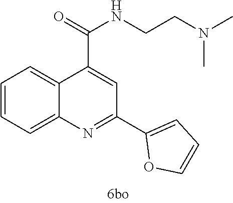 [SMILES: CN(C)CCNC(=O)c1cc(-c2ccco2)nc2ccccc12] | US/KY/14-18953 EC₅₀ = 1.1 ± 0.4 CC₅₀ = 198.6 ± 9.4 SI₅₀ = 180.5 US/MO/14-18947 EC₅₀ = 1.1 ± 0.1 CC₅₀ = 198.6 ± 9.4 SI₅₀ = 180.5 | Tainan EC₅₀ = 30.9 ± 1.3 CC₅₀ = 198.6 ± 9.4 SI₅₀ = 6.4 US/AK EC₅₀ = 20.9 ± 8.2 CC₅₀ = 198.6 ± 9.4 SI₅₀ = 9.5 | EC₅₀ = 1.2 ± 0.1 CC₅₀ >300.0 SI₅₀ >250.0 | N.T. | N.T. |

Antiviral efficacy was determined in the CPE assay. Cytotoxicity was determined using the neutral red method. The results are the mean ± standard deviation of three repeats.
SI = selectivity index (CC₅₀/EC₅₀). T_{1/2} was obtained using mouse liver microsomes.
N.A. = not tested.

TABLE 11
Broad-spectrum antiviral activity of known 2C inhibitors against non-polioentero viruses.

| | Antiviral activity and | Antiviral activity and | |
|---|---|---|---|
| | selectivity index against | selectivity index against | Antiviral activity and |
| | EV-D68 US/KY/14- | EV-A71 Tainan in RD | selectivity index against |
| Structure | 18953 in RD cells (μM) | cells (μM) | CVB3 in Vero cells (μM) |
| 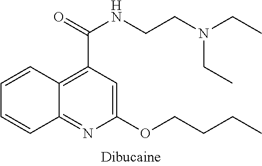 [SMILES: CCCCOc1cc(C(=O)NCCN(CC)CC)c2ccccc2n1] | EC₅₀ = 5.3 ± 1.2 CC₅₀ = 56.6 ± 28.7 SI₅₀ = 10.7 | EC₅₀ >10.0 CC₅₀ = 56.6 ± 28.7 SI₅₀ = N.A. | EC₅₀ = 1.3 ± 0.2 CC₅₀ = 64.4 ± 2.1 SI₅₀ = 49.5 |
| 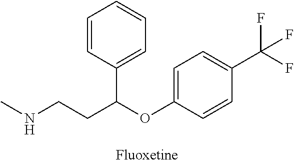 [SMILES: CNCCC(Oc1ccc(C(F)(F)F)cc1)c1ccccc1] | EC₅₀ = 1.0 ± 0.1 CC₅₀ = 11.9 ± 4.9 SI₅₀ = 11.9 | EC₅₀ >10.0 CC₅₀ = 11.9 ± 4.9 SI₅₀ = N.A. | EC₅₀ = 2.1 ± 1.0 CC₅₀ = 11.0 ± 3.1 SI₅₀ = 5.2 |
| 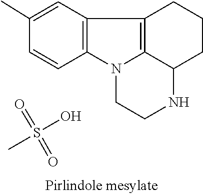 | EC₅₀ = 8.4 ± 1.1 CC₅₀ = 19.0 ± 1.6 SI₅₀ = 2.3 | EC₅₀ >20 CC₅₀ = 19.0 ± 1.6 SI₅₀ = N.A. | EC₅₀ = 3.6 ± 0.8 CC₅₀ = 40.5 ± 3.5 SI₅₀ = 11.3 |
| 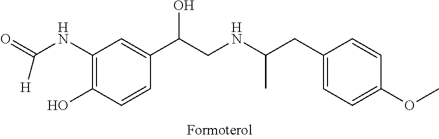 [SMILES: COc1ccc(CC(C)NCC(O)c2ccc(O)c(NC=O)c2)cc1] | EC₅₀ = 24.7 ± 1.1 CC₅₀ = 146.3 ± 27.8 S1₅₀ = 5.9 | EC₅₀ >100 CC₅₀ = 146.3 ± 27.8 SI₅₀ = N.A. | EC₅₀ = 9.9 ± 1.1 CC₅₀ >300.0 SI₅₀ >30.3 |
| 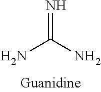 | EC₅₀ = 219.4 ± 15.0 CC₅₀ >1000.0 SI₅₀ >4.6 | EC₅₀ = 901.6 CC₅₀ >1000.0 SI₅₀ >1.1 | EC₅₀ = 224.5 ± 57.9 CC₅₀ >3000.0 SI₅₀ >13.4 |

Antiviral efficacy was determined in the CPE assay. Cytotoxicity was determined using the neutral red method. The results are the mean ± standard deviation of three repeats.
SI₅₀ = selectivity index (CC₅₀/EC₅₀).
N.A. = not applicable.

Next, selected lead compounds with potent and broad-spectrum antiviral activity against EV-D68, EV-A71, and CVB3 were profiled for mouse microsomal stability (see, Y. Wang, et al., J. Med. Chem. 61 (2018) 1074-1085; Y. Hu, et al., ACS Med. Chem. Lett. (2018), 9, 1111-1116). Compound 6af was included to investigate the influence of the terminal amine substitution on microsomal stability. In general, compounds with terminal monoalkylamine and dialkylamine had longer half-lives than compounds with trialkylamine (6af vs 6aa; 6aw, 6ax vs 6av; 6bj, 6b1 vs 6bf). Installing a fluorine at the 4-position of benzene indeed increased the microsomal stability (6aw vs 6af). Collectively, the most promising candidate is 6aw, which not only had potent antiviral activity against EV-D68, EV-A71 and CVB3 viruses, but also had a long half-life in mouse microsomes with T1/2 of 114.7 minutes.

### Antiviral Mechanism of Quinoline Analogs

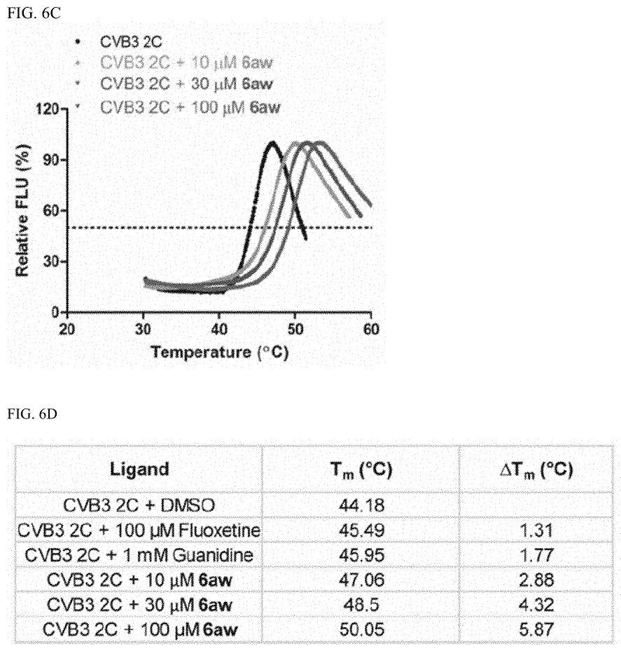

To provide direct evidence that the quinoline compounds target the viral 2C protein, we expressed the CVB3 2C protein and quantified the drug binding using the differential scanning fluorimetry (DSF), or thermal shift assay (TSA) (see, R. Musharrafieh, et al., J. Virol. 93 (2019), e02221-18; L. Bauer, et al., ACS Infect. Dis. 5 (2019) 1609-1623). In this assay, a temperature gradient is applied to unfold a given protein, and when the protein unfolds, it exposes the hydrophobic region that can bind to a fluorescence dye, resulting in increased fluorescence emission. When the protein is stabilized by a small molecule binder, the melting temperature, Tₘ, will increase. For this experiment, experiments were conducted that included two known 2C inhibitors, fluoxetine and guanidine as positive controls, and the most promising lead candidate 6aw was tested in three different concentrations. As shown in FIG. 6, CVB3 2C protein was stabilized by both fluoxetine and guanidine with Tₘ shifts of 1.31 and 1.77° C. when tested at 100 μM and 1 mM, respectively (FIGS. 6A&1B). Similarly, compound 6aw also increased the thermal stability of CVB3 2C in a concentration dependent manner and had a more profound effect than fluoxetine at 100 μM (FIGS. 6C&1D), which suggests that compound 6aw binds with a higher affinity than fluoxetine to 2C protein. This result is consistent with the higher antiviral efficacy of 6aw compared to fluoxetine. Overall, the TSA assay provided direct evidence that quinoline compound 6aw binds directly to the viral 2C protein and the binding affinity is correlated with the cellular antiviral efficacy.

### Conclusions

Non-polio enteroviruses are significant human pathogens for which we do not have any treatment available for now. More alarmingly, there have been an increasing trend of severe infections including EV-A71 and EV-D68 associated neurological complications such as acute flaccid myelitis in recently years (see, Francois-Moutal, L.; Wang, Y.; Moutal, A.; Cottier, K. E.; Melemedjian, O. K.; Yang, X.; Wang, Y.; Ju, W.; Largent-Milnes, T. M.; Khanna, M.; Vanderah, T. W.; Khanna, R. A membrane-delimited N-myristoylated CRMP2 peptide aptamer inhibits CaV2.2 trafficking and reverses inflammatory and postoperative pain behaviors. Pain 2015, 156, 1247-1264; Brittain, J. M.; et al., Nat. Med. 2011, 17, 822-829). Although the detailed mechanism regarding how these contemporary viruses lead to neurological infections is still under debate (see, A. B. Rosenfeld, et al., mBio, 10 (2019) e02370-02319; D. M. Brown, et al., MBio, 9 (2018), e01954-18), there is nevertheless a consensus among the scientific community that EV-A71 and EV-D68 are the etiological agents causing the neurological complications based on clinical data as well as animal model studies (see, R. D. Schubert, et al., Nature Med. 25 (2019) 1748-1752; N. Mishra, et al., MBio, 10 (2019), e01903-19). As such, developing effective antivirals against these non-polio enteroviruses is a valid approach to prevent and treat these viral infections. In this study, we aim to develop broad-spectrum antivirals with a high selectivity index as well as a long half-life in mouse microsomes. This is a multi-parameter optimization approach and the focus was on balancing different properties. To achieve this goal, experiments were conducted that adapted a stepwise optimization strategy. Specifically, experiments started with structure-activity relationship studies of a quinoline lead compound 6aa, and generated a number of candidates with potent antiviral activity and a high selectivity index against the EV-D68 US/KY/14-18953 virus. Next, 13 prioritized lead compounds were further profiled for broad-spectrum antiviral activity against an additional strain of EV-D68, the EV-D68 US/MO/14-18947 virus, as well as two EV-A71 viruses (Tainan/4643/98 and US/AK/16-19516) and one CVB3 virus. In parallel, compounds with potent and broad-spectrum antiviral activity were evaluated for mouse liver microsomal stability. Overall, this multi-parameter optimization strategy yielded the promising candidate 6aw that not only showed potent and broad-spectrum antiviral activity against all five strains of non-polio enteroviruses tested, but also displayed a high microsomal stability with a T112 of 114.7 minutes. In summary, this study is a step forward towards developing the urgently needed antivirals against non-polio enteroviruses, and the results presented herein might be informative in guiding other medicinal chemistry projects, such as optimization of microsomal stability.

### EXPERIMENTAL SECTION

### Chemistry

### General Chemical Methods

All chemicals were purchased from commercial vendors and used without further purification unless otherwise noted. ¹H and ¹³C NMR spectra were recorded on a Bruker-400 or -500 NMR spectrometer. Chemical shifts are reported in parts per million referenced with respect to residual solvent (CD₃OD) 3.31 ppm, (DMSO-d₆) 2.50 ppm, and (CDCl₃) 7.26 ppm or from internal standard tetramethylsilane (TMS) 0.00 ppm. The following abbreviations were used in reporting spectra: s, singlet; d, doublet; t, triplet; q, quartet; m, multiplet; dd, doublet of doublets; ddd, doublet of doublet of doublets. All reactions were carried out under N₂ atmosphere, unless otherwise stated. HPLC-grade solvents were used for all reactions. Flash column chromatography was performed using silica gel (230-400 mesh, Merck). Low-resolution mass spectra were obtained using an ESI technique on a 3200 Q Trap LC/MS/MS system (Applied Biosystems). The purity was assessed by using Shimadzu LC-MS with Waters XTerra MS C-18 column (part #186000538), 50×2.1 mm, at a flow rate of 0.3 mL/min; λ=250 and 220 nm; mobile phase A, 0.1% formic acid in H₂O, and mobile phase B′, 0.1% formic in 60% isopropanol, 30% CH₃CN and 9.9% H₂O. All compounds submitted for testing in TEVC assay and plaque reduction assay were confirmed to be >95.0% purity by LC-MS traces. All compounds were characterized by proton and carbon NMR and MS.

### General Procedures for the Synthesis of Compounds 3 by Suzuki Coupling

To a solution of 2-chloroquinoline-4-carboxylic acid (1) (1 equiv) in dioxane (4 mL) in a microwave reaction vial was added boronic acid (1.2 equiv) and aqueous potassium carbonate solution (3 equiv in 1 mL). The mixture was purged with dry nitrogen for 5 min. Then tetrakis(triphenylphosphine)palladium(0) (0.5 equiv) was added and the solution was heated in the biotage microwave reactor at 140° C. for 30 min. The reaction mixture was diluted with dichloromethane and extracted with aqueous NaHCO₃ solution and brine. The organic layer was separated, dried over anhydrous magnesium sulfate, filtered, and concentrated under reduced pressure. The mixture was then purified by silica gel flash column chromatography (0-10% CH₃OH/CH₂Cl₂) to give the final product.

### General Procedures for the Synthesis of Compounds 5 by HATU Coupling

To a solution of quinoline 4-carboxylic acid (3) (1 equiv) in DMF was added amine (1 equiv) and DIEA (1 equiv). Then HATU (1 equiv) was added in one portion and the mixture was stirred at room temperature overnight. The reaction mixture was diluted with dichloromethane and extracted with aqueous NaHCO₃ solution and brine. The organic layer was separated, dried over anhydrous magnesium sulfate, filtered, and concentrated under reduced pressure. The mixture was then purified by silica gel flash column chromatography (0-10% CH₃OH/CH₂Cl₂) to give the final product.

### General Procedures for the Synthesis of Compounds 6 by TFA Deprotection

To a solution of quinoline 4-amide (5) (1 equiv) in dichloromethane (5 mL) was added TFA (1 mL). The mixture was stirred at room temperature for 2 hrs, and the solvent was removed under reduced pressure. The crude mixture was diluted in dichloromethane and purified by silica gel flash column chromatography (0-10% CH₃OH/CH₂Cl₂) to give the final product.

The synthesis and characterization for compounds 6aa and 6bf were previously reported (see, R. Musharrafieh, et al., J. Med. Chem. 62 (2019) 4074-4090).

### 2-(dimethylamino)ethyl 2-phenylquinoline-4-carboxylate (6ab)

Yield: 75.1%. ¹H NMR (400 MHz, DMSO-d₆) δ 8.58 (dd, J=8.6, 1.4 Hz, 1H), 8.42 (s, 1H), 8.31-8.21 (m, 2H), 8.20-8.12 (m, 1H), 7.84 (ddd, J=8.4, 6.9, 1.4 Hz, 1H), 7.69 (ddd, J=8.3, 6.8, 1.3 Hz, 1H), 7.61-7.48 (m, 3H), 4.54 (t, J=5.7 Hz, 2H), 2.74 (t, J=5.7 Hz, 2H), 2.28 (s, 6H). ¹³C NMR (101 MHz, DMSO-d₆) δ 165.82, 155.69, 148.30, 137.73, 136.68, 130.35, 130.01, 129.79, 128.96, 127.93, 127.14, 125.17, 123.09, 119.10, 63.19, 57.02, 45.18. C₂₀H₂₀N₂O₂, EI-MS: m/z (M+H⁺): 321.4 (calculated), 321.6 (found).

### N-[3-(methylamino)propyl]-2-phenylquinoline-4-carboxamide (6ac)

Yield: 68.5%. ¹H NMR (400 MHz, DMSO-d₆) δ 9.04 (t, J=5.7 Hz, 1H), 8.87-8.64 (m, 2H), 8.38-8.27 (m, 2H), 8.25-8.07 (m, 3H), 7.83 (ddd, J=8.4, 6.8, 1.4 Hz, 1H), 7.65 (ddd, J=8.3, 6.8, 1.3 Hz, 1H), 7.62-7.45 (m, 3H), 3.46 (q, J=6.5 Hz, 2H), 3.10-2.97 (m, 2H), 2.61 (t, J=5.4 Hz, 3H), 1.95 (p, J=6.9 Hz, 2H). ¹³C NMR (101 MHz, DMSO-d₆) δ 166.94, 155.80, 147.94, 142.87, 138.20, 130.21, 129.92, 129.56, 128.91, 127.31, 127.16, 125.34, 123.35, 116.75, 46.26, 36.41, 32.49, 25.79. C₂₀H₂₁N₃O, EI-MS: m/z (M+H⁺): 320.4 (calculated), 320.4 (found).

### N-(3-aminopropyl)-2-phenylquinoline-4-carboxamide (6ad)

Yield: 80.3%. ¹H NMR (400 MHz, DMSO-d₆) δ 9.04 (t, J=5.7 Hz, 1H), 8.39-8.26 (m, 2H), 8.26-8.09 (m, 3H), 8.06-7.88 (br s, 3H), 7.83 (ddd, J=8.4, 6.9, 1.4 Hz, 1H), 7.64 (ddd, J=8.3, 6.9, 1.3 Hz, 1H), 7.62-7.43 (m, 3H), 3.47 (q, J=6.5 Hz, 2H), 2.97 (q, J=6.8 Hz, 2H), 1.93 (p, J=6.9 Hz, 2H). ¹³C NMR (101 MHz, DMSO-d₆) δ 167.00, 155.84, 147.97, 142.96, 138.23, 130.25, 129.96, 129.59, 128.95, 127.33, 127.19, 125.34, 123.39, 116.76, 48.63, 37.02, 36.45, 27.38. C₁₉H₁₉N₃O, EI-MS: m/z (M+H⁺): 306.4 (calculated), 306.4 (found).

### N-[2-(1H-imidazol-1-yl)ethyl]-2-phenylquinoline-4-carboxamide (6ae)

Yield: 89.2%. ¹H NMR (400 MHz, DMSO-d₆) δ 8.97 (t, J=5.7 Hz, 1H), 8.35-8.22 (m, 2H), 8.12 (dd, J=8.6, 1.2 Hz, 1H), 8.07-7.98 (m, 2H), 7.81 (ddd, J=8.4, 6.8, 1.5 Hz, 1H), 7.71 (s, 1H), 7.65-7.47 (m, 4H), 7.27 (s, 1H), 6.95 (s, 1H), 4.26 (t, J=6.6, 5.2 Hz, 2H), 3.73 (q, J=5.8 Hz, 2H). ¹³C NMR (101 MHz, DMSO-d₆) δ 166.95, 155.67, 147.87, 142.87, 138.15, 137.62, 130.20, 129.91, 129.45, 128.92, 128.41, 127.19, 127.10, 125.34, 123.22, 119.66, 116.62, 45.20, 40.15. C₂₂H₁₈N₄O, EI-MS: m/z (M+H⁺): 343.4 (calculated), 343.2 (found).

### N-[2-(methylamino)ethyl]-2-phenylquinoline-4-carboxamide (6af)

Yield: 72.3%. ¹H NMR (400 MHz, DMSO-d₆) δ 9.15 (t, J=5.6 Hz, 1H), 8.80 (s, 2H), 8.36-8.23 (m, 4H), 8.14 (dd, J=8.6, 1.2 Hz, 1H), 7.83 (ddd, J=8.4, 6.8, 1.5 Hz, 1H), 7.65 (ddd, J=8.3, 6.8, 1.3 Hz, 1H), 7.62-7.41 (m, 3H), 3.69 (q, J=6.0 Hz, 2H), 3.22 (p, J=6.1 Hz, 2H), 2.68 (t, J=5.3 Hz, 3H). ¹³C NMR (101 MHz, DMSO-d₆) δ 167.34, 155.78, 148.00, 142.15, 138.19, 130.22, 129.98, 129.52, 128.94, 127.29, 127.14, 125.61, 123.32, 117.20, 47.69, 40.15, 35.80, 32.71. C₁₉H₁₉N₃O, EI-MS: m/z (M+H⁺): 306.4 (calculated), 306.7 (found).

### N-[(azetidin-3-yl)methyl]-2-phenylquinoline-4-carboxamide (6ag)

Yield: 74.3%. ¹H NMR (400 MHz, DMSO-d₆) δ 9.13 (t, J=5.8 Hz, 1H), 8.95 (d, J=52.3 Hz, 2H), 8.40-8.28 (m, 2H), 8.24-8.16 (m, 2H), 8.14 (ddd, J=8.5, 1.3, 0.6 Hz, 1H), 7.83 (ddd, J=8.4, 6.9, 1.4 Hz, 1H), 7.65 (ddd, J=8.2, 6.8, 1.3 Hz, 1H), 7.61-7.45 (m, 3H), 4.16-3.97 (m, 3H), 3.97-3.81 (m, 2H), 3.62 (t, J=6.2 Hz, 2H), 3.17-3.07 (m, 1H). ¹³C NMR (101 MHz, DMSO-d₆) δ 167.33, 155.80, 147.94, 142.68, 138.19, 130.21, 129.93, 129.56, 128.91, 127.33, 127.20, 125.36, 123.30, 116.86, 48.66, 48.60, 40.84, 31.74. C₂₀H₁₉N₃O, EI-MS: m/z (M+H⁺): 318.4 (calculated), 318.4 (found).

### N-[(3-hydroxyazetidin-3-yl)methyl]-2-phenylquinoline-4-carboxamide (6ah)

Yield: 83.1%. ¹H NMR (400 MHz, DMSO-d₆) δ 9.29-9.13 (m, 2H), 9.07 (s, 1H), 8.39-8.29 (m, 2H), 8.29-8.18 (m, 2H), 8.14 (ddd, J=8.4, 1.3, 0.7 Hz, 1H), 7.82 (ddd, J=8.4, 6.8, 1.4 Hz, 1H), 7.64 (ddd, J=8.3, 6.9, 1.3 Hz, 1H), 7.62-7.45 (m, 3H), 4.23-4.07 (m, 3H), 3.97-3.80 (m, 3H), 3.70 (d, J=6.0 Hz, 2H). ¹³C NMR (101 MHz, DMSO-d₆) δ 167.74, 155.80, 147.95, 142.58, 138.27, 130.17, 129.92, 129.54, 128.91, 127.36, 127.14, 125.52, 123.39, 117.08, 71.36, 56.21, 48.62, 44.93. C₂₀H₁₉N₃O₂, EI-MS: m/z (M+H⁺): 334.4 (calculated), 334.2 (found).

### 2-phenyl-N-[2-(pyrrolidin-2-yl)ethyl]quinoline-4-carboxamide (6ai)

Yield: 68.3%. ¹H NMR (400 MHz, DMSO-d₆) δ 9.29 (s, 1H), 9.04 (t, J=5.6 Hz, 1H), 8.76 (s, 1H), 8.37-8.26 (m, 2H), 8.21 (ddd, J=8.4, 1.5, 0.6 Hz, 1H), 8.19-8.05 (m, 2H), 7.83 (ddd, J=8.4, 6.8, 1.4 Hz, 1H), 7.65 (ddd, J=8.3, 6.9, 1.3 Hz, 1H), 7.61-7.42 (m, 3H), 3.66-3.52 (m, 1H), 3.52-3.39 (m, 2H), 3.28-3.17 (m, 2H), 2.30-2.15 (m, 1H), 2.15-2.03 (m, 1H), 2.03-1.78 (m, 3H), 1.71-1.49 (m, 1H). ¹³C NMR (101 MHz, DMSO-d₆) δ 166.87, 155.80, 147.95, 142.86, 138.20, 130.21, 129.92, 129.56, 128.92, 127.30, 127.16, 125.35, 123.35, 116.72, 57.58, 44.33, 36.67, 31.43, 29.57, 22.99. C₂₂H₂₃N₃O, EI-MS: m/z (M+H⁺): 346.4 (calculated), 346.4 (found).

### N-(azetidin-3-yl)-2-phenylquinoline-4-carboxamide (6aj)

Yield: 88.4%. ¹H NMR (400 MHz, DMSO-d₆) δ 9.64 (d, J=6.7 Hz, 1H), 9.14 (s, 2H), 8.39-8.30 (m, 2H), 8.30-8.20 (m, 2H), 8.20-8.08 (m, 1H), 7.84 (ddd, J=8.4, 6.9, 1.5 Hz, 1H), 7.65 (ddd, J=8.2, 6.8, 1.3 Hz, 1H), 7.62-7.42 (m, 3H), 5.08-4.82 (m, 1H), 4.37-4.25 (m, 2H), 4.25-4.05 (m, 2H). ¹³C NMR (101 MHz, DMSO-d₆) δ 166.53, 155.76, 147.97, 141.85, 138.08, 130.32, 130.00, 129.60, 128.93, 127.30, 127.26, 125.28, 123.14, 117.04, 51.93, 41.63. C₁₉H₁₇N₃O, EI-MS: m/z (M+H⁺): 304.4 (calculated), 304.3 (found).

### N-(1-methylazetidin-3-yl)-2-phenylquinoline-4-carboxamide (6ak)

Yield: 92.3%. ¹H NMR (400 MHz, DMSO-d₆) δ 9.45 (d, J=6.9 Hz, 1H), 8.41-8.28 (m, 2H), 8.26-8.16 (m, 2H), 8.13 (ddd, J=8.4, 1.3, 0.7 Hz, 1H), 7.82 (ddd, J=8.4, 6.9, 1.5 Hz, 1H), 7.64 (ddd, J=8.3, 6.9, 1.3 Hz, 1H), 7.61-7.45 (m, 3H), 4.66 (h, J=7.0 Hz, 1H), 3.87 (td, J=7.2, 1.8 Hz, 2H), 3.40 (td, J=6.9, 1.6 Hz, 2H), 2.45 (s, 3H). ¹³C NMR (101 MHz, DMSO-d₆) δ 166.22, 155.75, 147.91, 142.35, 138.16, 130.19, 129.91, 129.54, 128.89, 127.31, 127.18, 125.27, 123.28, 116.90, 62.00, 48.58, 44.42. C₂₀H₁₉N₃O, EI-MS: m/z (M+H⁺): 318.4 (calculated), 318.4 (found).

### N-[(3-methylazetidin-3-yl)methyl]-2-phenylquinoline-4-carboxamide (6al)

Yield: 71.6%. ¹H NMR (400 MHz, CD₃OD) δ 8.24-8.14 (m, 4H), 8.10 (s, 1H), 7.81 (ddd, J=8.5, 6.9, 1.4 Hz, 1H), 7.64 (ddd, J=8.2, 6.9, 1.3 Hz, 1H), 7.61-7.44 (m, 3H), 4.18 (d, J=11.0 Hz, 2H), 3.88-3.78 (m, 2H), 3.71 (s, 2H), 3.35 (s, 3H). ¹³C NMR (101 MHz, CD₃OD) δ 170.86, 158.50, 149.50, 144.26, 139.85, 131.62, 131.05, 130.31, 130.00, 128.75, 128.58, 126.17, 124.69, 118.39, 55.69, 49.85, 47.19, 39.79, 22.57. C₂₁H₂₁N₃O, EI-MS: m/z (M+H⁺): 332.4 (calculated), 332.4 (found).

### 2-phenyl-N-[(piperidin-3-yOmethyl]quinoline-4-carboxamide (6 am)

Yield: 76.3%. ¹H NMR (400 MHz, DMSO-d₆) δ 9.04 (t, J=6.0 Hz, 1H), 8.99-8.87 (m, 1H), 8.75-8.55 (m, 1H), 8.36-8.27 (m, 2H), 8.23-8.09 (m, 3H), 7.83 (ddd, J=8.4, 6.9, 1.5 Hz, 1H), 7.64 (ddd, J=8.3, 6.9, 1.3 Hz, 1H), 7.62-7.44 (m, 3H), 3.47-3.33 (m, 2H), 3.33-3.21 (m, 2H), 2.93-2.77 (m, 1H), 2.77-2.61 (m, 1H), 2.18-2.01 (m, 1H), 1.94-1.77 (m, 2H), 1.75-1.54 (m, 1H), 1.41-1.22 (m, 1H). ¹³C NMR (101 MHz, DMSO-d₆) δ 167.02, 155.81, 147.95, 142.89, 138.22, 130.21, 129.93, 129.58, 128.92, 127.32, 127.18, 125.32, 123.36, 116.74, 46.45, 43.37, 42.03, 33.96, 26.09, 21.56. C₂₂H₂₃N₃O, EI-MS: m/z (M+H⁺): 346.4 (calculated), 346.4 (found).

### N-(2-aminoethyl)-2-phenylquinoline-4-carboxamide (6an)

Yield: 75.3%. ¹H NMR (400 MHz, DMSO-d₆) δ 9.14 (t, J=5.6 Hz, 1H), 8.41-8.31 (m, 3H), 8.28 (dd, J=8.5, 1.3 Hz, 1H), 8.24-8.05 (m, 4H), 7.82 (ddd, J=8.4, 6.8, 1.4 Hz, 1H), 7.64 (ddd, J=8.3, 6.8, 1.3 Hz, 1H), 7.61-7.43 (m, 3H), 3.66 (q, J=6.1 Hz, 2H), 3.13 (q, J=5.9 Hz, 2H). NMR (101 MHz, DMSO-d₆) δ 167.31, 155.81, 148.00, 142.27, 138.23, 130.19, 129.96, 129.53, 128.92, 127.32, 127.13, 125.60, 123.35, 117.23, 38.43, 37.22. C₁₈H₁₇N₃O, EI-MS: m/z (M+H⁺): 292.4 (calculated), 292.4 (found).

### N-(2-carbamimidamidoethyl)-2-phenylquinoline-4-carboxamide (6ao)

Yield: 73.2%. ¹H NMR (400 MHz, CD₃OD) δ 8.26-8.11 (m, 4H), 8.08 (s, 1H), 7.80 (ddd, J=8.4, 6.9, 1.4 Hz, 1H), 7.62 (ddd, J=8.3, 6.9, 1.3 Hz, 1H), 7.59-7.45 (m, 3H), 3.68 (t, J=6.6, 5.5 Hz, 2H), 3.53 (t, J=6.6, 5.5 Hz, 2H). ¹³C NMR (101 MHz, CD₃OD) δ 170.59, 159.04, 158.50, 149.54, 144.21, 139.89, 131.59, 131.02, 130.26, 129.99, 128.73, 128.52, 126.30, 124.72, 118.44, 41.91, 40.02. C₁₉H₁₉N₅O, EI-MS: m/z (M+H⁺): 334.4 (calculated), 334.4 (found).

### N-(1-aminopropan-2-yl)-2-phenylquinoline-4-carboxamide (6ap)

Yield: 89.6%. ¹H NMR (400 MHz, DMSO-d₆) δ 8.94 (d, J=8.0 Hz, 1H), 8.40-8.30 (m, 3H), 8.26 (dd, J=8.5, 1.4 Hz, 1H), 8.23-8.03 (m, 4H), 7.84 (ddd, J=8.4, 6.9, 1.4 Hz, 1H), 7.66 (ddd, J=8.3, 6.8, 1.3 Hz, 1H), 7.63-7.40 (m, 3H), 4.54-4.34 (m, 1H), 3.14-2.96 (m, 2H), 1.31 (d, J=6.7 Hz, 3H). ¹³C NMR (101 MHz, DMSO-d₆) δ 166.81, 155.77, 147.95, 142.47, 138.24, 130.16, 129.95, 129.53, 128.92, 127.30, 127.12, 125.51, 123.35, 117.15, 43.75, 43.23, 18.12. C₁₉H₁₉N₃O, EI-MS: m/z (M+H⁺): 306.4 (calculated), 306.5 (found).

### N-[1-(dimethylamino)propan-2-yl]-2-phenylquinoline-4-carboxamide (6aq)

Yield: 94.3%. ¹H NMR (400 MHz, DMSO-d₆) δ 8.63 (d, J=8.3 Hz, 1H), 8.37-8.20 (m, 3H), 8.13 (dd, J=8.6, 1.2 Hz, 1H), 8.08 (s, 1H), 7.82 (ddd, J=8.4, 6.8, 1.4 Hz, 1H), 7.64 (ddd, J=8.3, 6.8, 1.3 Hz, 1H), 7.61-7.43 (m, 3H), 4.40-4.17 (m, 1H), 2.49-2.40 (m, 1H), 2.34-2.17 (m, 7H), 1.22 (d, J=6.6 Hz, 3H). ¹³C NMR (101 MHz, DMSO-d₆) δ 165.96, 155.71, 147.88, 143.76, 138.26, 130.10, 129.81, 129.45, 128.88, 127.24, 127.00, 125.53, 123.45, 116.38, 64.41, 45.49, 43.18, 18.83. C₂₁H₂₃N₃O, EI-MS: m/z (M+H⁺): 334.4 (calculated), 334.4 (found).

### N-[2-(dimethylamino)ethyl]-2 [(2, 3,4,5,6-²H₅)phenyl]quinoline-4-carboxamide (6ar)

Yield: 87.3%. ¹H NMR (400 MHz, DMSO-d₆) δ 8.81 (t, J=5.7 Hz, 1H), 8.30-8.19 (m, 1H), 8.19-8.06 (m, 2H), 7.82 (ddd, J=8.4, 6.8, 1.4 Hz, 1H), 7.64 (ddd, J=8.3, 6.8, 1.3 Hz, 1H), 3.49 (q, J=6.5 Hz, 2H), 2.58-2.48 (m, 3H), 2.25 (s, 6H). ¹³C NMR (101 MHz, DMSO-d₆) δ 166.61, 155.71, 147.90, 143.33, 138.07, 130.11, 129.48, 127.03, 125.46, 123.42, 116.54, 58.00, 45.19, 37.34. C₂₀H₁₆D₅N₃O, EI-MS: m/z (M+H⁺): 325.4 (calculated), 325.4 (found).

### N-[2-(methylamino)ethyl]-2 [(2, 3, 4, 5, 6-²H₅)phenyl]quinoline-4-carboxamide (6as)

Yield: 71.2%. ¹H NMR (400 MHz, DMSO-d₆) δ 9.19 (t, J=5.6 Hz, 1H), 8.95 (s, 2H), 8.36-8.25 (m, 2H), 8.13 (ddd, J=8.5, 1.3, 0.7 Hz, 1H), 7.88-7.77 (m, 1H), 7.64 (ddd, J=8.3, 6.9, 1.3 Hz, 1H), 3.75-3.64 (m, 2H), 3.28-3.18 (m, 2H), 2.72-2.58 (m, 3H). ¹³C NMR (101 MHz, DMSO-d₆) δ 167.34, 155.80, 148.04, 142.13, 138.07, 130.20, 129.54, 127.12, 125.63, 123.36, 117.26, 47.64, 35.81, 32.65. C₁₉H₁₄D₅N₃O, EI-MS: m/z (M+H⁺): 311.4 (calculated), 311.4 (found).

### N-(2-aminoethyl)-2 [(2, 3, 4, 5, 6-²H₅)phenyl]quinoline-4-carboxamide (6at)

Yield: 69.6%. ¹H NMR (400 MHz, DMSO-d₆) δ 7.45 (dd, J=8.5, 1.3 Hz, 1H), 7.38 (s, 1H), 7.34 (dd, J=8.4, 1.0 Hz, 1H), 6.99 (ddd, J=8.4, 6.8, 1.4 Hz, 1H), 6.81 (ddd, J=8.3, 6.9, 1.3 Hz, 1H), 2.99 (t, J=6.0 Hz, 2H), 2.49-2.44 (m, 2H). ¹³C NMR (101 MHz, DMSO-d₆) δ 161.39, 149.02, 140.27, 134.15, 130.39, 121.97, 120.92, 118.94, 116.90, 115.22, 109.20, 31.20, 29.54. C₁₈H₁₂D₅N₃O, EI-MS: m/z (M+H⁺): 297.4 (calculated), 297.4 (found).

### N-[(azetidin-3-yl)methyl]-2 [(2, 3,4,5,6-²H₅)phenyl]quinoline-4-carboxamide (6au)

Yield: 73.3%. ¹H NMR (400 MHz, DMSO-d₆) δ 9.37-9.02 (m, 2H), 8.23 (s, 1H), 8.18 (dd, J=8.5, 1.3 Hz, 1H), 8.16-8.08 (m, 1H), 7.82 (ddd, J=8.4, 6.8, 1.5 Hz, 1H), 7.64 (ddd, J=8.3, 6.9, 1.3 Hz, 1H), 4.10-3.97 (m, 2H), 3.95-3.80 (m, 2H), 3.61 (t, J=6.1 Hz, 2H), 3.19-3.11 (m, 1H). ¹³C NMR (101 MHz, DMSO-d₆) δ 167.37, 158.02, 155.83, 147.97, 142.67, 138.06, 130.21, 129.57, 127.20, 125.41, 123.35, 116.96, 115.79, 48.40, 40.86, 31.74. C₂₀H₁₄D₅N₃O, EI-MS: m/z (M+H⁺): 323.4 (calculated), 323.4 (found).

### N-[2-(dimethylamino)ethyl]-2-(4-fluorophenyl)quinoline-4-carboxamide (6av)

Yield: 86.6%. ¹H NMR (400 MHz, DMSO-d₆) δ 8.81 (t, J=5.7 Hz, 1H), 8.43-8.30 (m, 2H), 8.23 (dd, J=8.4, 1.4 Hz, 1H), 8.16-8.06 (m, 2H), 7.86-7.76 (m, 1H), 7.70-7.48 (m, 2H), 7.44-7.33 (m, 2H), 3.50 (q, J=6.4 Hz, 2H), 2.55 (t, J=6.7 Hz, 2H), 2.27 (s, 6H). ¹³C NMR (101 MHz, DMSO-d₆) δ 166.57, 164.58, 162.13, 154.68, 147.82, 143.39, 134.75, 134.72, 131.50, 131.41, 130.18, 129.59, 129.50, 129.41, 128.78, 128.66, 127.07, 125.46, 123.32, 116.40, 115.89, 115.68, 57.92, 45.10, 37.26. C₂₀H₂₀FN₃O, EI-MS: m/z (M+H⁺): 338.4 (calculated), 338.4 (found).

### 2-(4 fluorophenyl)-N-[2-(methylamino)ethyl]quinoline-4-carboxamide (6aw)

Yield: 88.3%. ¹H NMR (400 MHz, DMSO-d₆) δ 9.18 (t, J=5.6 Hz, 1H), 8.93 (s, 2H), 8.48-8.35 (m, 2H), 8.35-8.23 (m, 2H), 8.19-8.06 (m, 1H), 7.82 (ddd, J=8.4, 6.8, 1.4 Hz, 1H), 7.64 (ddd, J=8.2, 6.8, 1.3 Hz, 1H), 7.48-7.32 (m, 2H), 3.69 (q, J=6.0 Hz, 2H), 3.29-3.19 (m, 2H), 2.67 (s, 3H). ¹³C NMR (101 MHz, DMSO-d₆) δ 167.22, 164.64, 162.18, 154.71, 147.93, 142.16, 134.72, 134.69, 130.24, 129.62, 129.54, 129.46, 127.13, 125.59, 123.23, 117.08, 115.93, 115.72, 47.60, 35.77, 32.63. C₁₉H₁₈FN₃O, EI-MS: m/z (M+H⁺): 324.4 (calculated), 324.4 (found).

### N-(2-aminoethyl)-2-(4-fluorophenyl)quinoline-4-carboxamide (6ax)

Yield: 85.3%. ¹H NMR (400 MHz, DMSO-d₆) δ 9.16 (t, J=5.6 Hz, 1H), 8.44-8.36 (m, 2H), 8.33 (s, 1H), 8.30-8.25 (m, 1H), 8.20 (s, 3H), 8.14-8.08 (m, 1H), 7.82 (ddd, J=8.4, 6.8, 1.4 Hz, 1H), 7.64 (ddd, J=8.3, 6.8, 1.3 Hz, 1H), 7.46-7.35 (m, 2H), 3.65 (q, J=6.1 Hz, 2H), 3.12 (t, J=6.2 Hz, 2H). ¹³C NMR (101 MHz, DMSO-d₆) δ 167.20, 164.65, 162.19, 154.75, 147.93, 142.29, 134.76, 134.73, 130.23, 129.66, 129.57, 129.47, 127.14, 125.59, 123.26, 117.10, 115.93, 115.72, 38.39, 37.20. C₁₈H₁₆FN₃O, EI-MS: m/z (M+H⁺): 310.3 (calculated), 310.3 (found).

### N-(azetidin-3-yl)-2-(4-fluorophenyl)quinoline-4-carboxamide (6ay)

Yield: 71.3%. ¹H NMR (400 MHz, DMSO-d₆) δ 9.70 (d, J=6.7 Hz, 1H), 9.26 (s, 2H), 8.46-8.34 (m, 2H), 8.28 (s, 1H), 8.23 (dd, J=8.5, 1.3 Hz, 1H), 8.18-8.08 (m, 1H), 7.83 (ddd, J=8.4, 6.9, 1.5 Hz, 1H), 7.65 (ddd, J=8.3, 6.9, 1.3 Hz, 1H), 7.48-7.33 (m, 2H), 5.03-4.86 (m, 1H), 4.36-4.23 (m, 2H), 4.23-4.10 (m, 2H). ¹³C NMR (101 MHz, DMSO-d₆) δ 166.45, 164.67, 162.21, 154.72, 147.90, 141.91, 134.63, 134.60, 130.38, 129.63, 129.54, 127.33, 125.30, 123.08, 116.94, 115.96, 115.75, 51.85, 41.62. C₁₉H₁₆FN₃O, EI-MS: m/z (M+H⁺): 322.4 (calculated), 322.4 (found).

### N-[(azetidin-3-yl)methyl]-2-(4-fluorophenyl)quinoline-4-carboxamide (6az)

Yield: 80.3%. ¹H NMR (400 MHz, DMSO-d₆) δ 9.27-8.88 (m, 3H), 8.48-8.33 (m, 2H), 8.23 (s, 1H), 8.21-8.15 (m, 1H), 8.15-8.05 (m, 1H), 7.82 (ddd, J=8.4, 6.8, 1.4 Hz, 1H), 7.65 (ddd, J=8.2, 6.9, 1.3 Hz, 1H), 7.46-7.34 (m, 2H), 4.12-3.96 (m, 2H), 3.96-3.77 (m, 2H), 3.62 (t, J=6.1 Hz, 2H), 3.23-3.01 (m, 1H). ¹³C NMR (101 MHz, DMSO-d₆) δ 167.28, 164.63, 162.18, 154.74, 147.86, 142.75, 134.70, 134.68, 130.26, 129.69, 129.61, 129.49, 127.21, 125.36, 123.21, 116.74, 115.90, 115.69, 48.50, 40.80, 31.72. C₂₀H₁₈FN₃O, EI-MS: m/z (M+H⁺): 336.4 (calculated), 336.4 (found).

### 2-(2,4-difluorophenyl)-N-[2-(dimethylamino)ethyl]quinoline-4-carboxamide (6ba)

Yield: 94.6%. ¹H NMR (400 MHz, DMSO-d₆) δ 8.77 (t, J=5.7 Hz, 1H), 8.21 (dd, J=8.4, 1.4 Hz, 1H), 8.18-8.05 (m, 2H), 7.89-7.76 (m, 2H), 7.68 (ddd, J=8.3, 6.8, 1.3 Hz, 1H), 7.46 (ddd, J=11.7, 9.2, 2.6 Hz, 1H), 7.36-7.23 (m, 1H), 3.46 (q, J=6.4 Hz, 2H), 2.54-2.49 (m, 2H), 2.24 (s, 6H). ¹³C NMR (101 MHz, DMSO-d₆) δ 166.39, 164.31, 164.19, 161.83, 161.71, 161.64, 161.51, 159.14, 159.01, 152.07, 152.04, 147.89, 143.29, 132.79, 132.75, 132.69, 132.65, 130.33, 129.46, 127.59, 125.44, 123.55, 123.46, 123.27, 119.38, 119.31, 112.49, 112.45, 112.28, 112.24, 105.08, 104.81, 104.55, 57.92, 45.12, 37.29. C₂₀H₁₉F₂N₃O, EI-MS: m/z (M+H⁺): 356.4 (calculated), 356.4 (found).

### 2-(2,6-difluorophenyl)-N-[2-(dimethylamino)ethyl]quinoline-4-carboxamide (6bb)

Yield: 86.5%. ¹H NMR (400 MHz, DMSO-d₆) δ 8.81 (t, J=5.7 Hz, 1H), 8.23 (ddd, J=25.1, 8.4, 1.4 Hz, 1H), 8.16-7.99 (m, 1H), 7.88 (ddd, J=8.4, 6.9, 1.5 Hz, 1H), 7.79-7.70 (m, 1H), 7.70-7.67 (m, 1H), 7.67-7.56 (m, 1H), 7.38-7.21 (m, 2H), 3.48 (q, J=6.5 Hz, 2H), 2.54-2.46 (m, 2H), 2.27 (s, 6H). ¹³C NMR (101 MHz, DMSO-d₆) δ 166.12, 158.47, 149.03, 147.81, 143.18, 131.58, 130.38, 129.40, 127.97, 127.85, 125.78, 125.54, 123.39, 120.62, 119.98, 112.27, 112.21, 112.08, 112.02, 57.83, 45.04, 37.21. C₂₀H₁₉F₂N₃O, EI-MS: m/z (M+H⁺): 356.4 (calculated), 356.4 (found).

### N-[2-(dimethylamino)ethyl]-2-(2,4,6-trifluorophenyl)quinoline-4-carboxamide (6bc)

Yield: 81.3%. ¹H NMR (400 MHz, DMSO-d₆) δ 8.88 (t, J=5.7 Hz, 1H), 8.17-8.06 (m, 1H), 7.78-7.61 (m, 2H), 7.61-7.52 (m, 1H), 7.52-7.33 (m, 3H), 3.48 (q, J=6.3 Hz, 2H), 2.57 (t, J=6.8 Hz, 2H), 2.29 (s, 6H). ¹³C NMR (101 MHz, DMSO-d₆) δ 165.66, 158.76, 147.61, 145.47, 130.82, 127.37, 126.00, 125.68, 122.58, 109.39, 101.69, 57.67, 44.90, 37.02. C₂₀H₁₈F₃N₃O, EI-MS: m/z (M+H⁺): 374.4 (calculated), 374.6 (found).

### N-[2-(dimethylamino)ethyl]-2-(3,4,5-trifluorophenyl)quinoline-4-carboxamide (6bd)

Yield: 72.9%. ¹H NMR (400 MHz, DMSO-d₆) δ 8.77 (t, J=5.7 Hz, 1H), 8.34-8.17 (m, 4H), 8.12 (dd, J=8.6, 1.2 Hz, 1H), 7.83 (ddd, J=8.4, 6.8, 1.4 Hz, 1H), 7.67 (ddd, J=8.3, 6.8, 1.3 Hz, 1H), 7.64-7.47 (m, 1H), 3.49 (q, J=6.5 Hz, 2H), 2.53 (t, J=6.8 Hz, 2H), 2.26 (s, 6H). ¹³C NMR (101 MHz, DMSO-d₆) δ 166.32, 152.24, 151.91, 149.56, 149.42, 147.57, 143.81, 134.90, 132.00, 131.98, 131.50, 131.40, 130.45, 129.53, 128.77, 128.65, 127.69, 125.47, 123.70, 116.27, 111.83, 111.77, 111.67, 111.61, 57.97, 45.16, 37.35. C₂₀H₁₈F₃N₃O, EI-MS: m/z (M+H⁺): 374.4 (calculated), 374.4 (found).

### N-[2-(dimethylamino)ethyl]-2-(2,3,4,5,6-pentafluorophenyl)quinoline-4-carboxamide (6be)

Yield: 85.3%. ¹H NMR (400 MHz, DMSO-d₆) δ 8.82 (t, J=5.7 Hz, 1H), 8.17 (dd, J=8.4, 1.4 Hz, 1H), 8.00 (dd, J=8.5, 1.2 Hz, 1H), 7.86 (ddd, J=8.4, 6.9, 1.5 Hz, 1H), 7.70 (ddd, J=8.3, 6.9, 1.3 Hz, 1H), 7.59 (s, 1H), 3.44 (q, J=6.4 Hz, 2H), 2.57-2.43 (m, 2H), 2.24 (s, 6H). ¹³C NMR (101 MHz, DMSO-d₆) δ 165.06, 149.30, 147.56, 146.01, 131.22, 128.25, 127.86, 125.77, 123.35, 119.96, 57.79, 45.06, 37.22. C₂₀H₁₆F₅N₃O, EI-MS: m/z (M+H⁺): 410.4 (calculated), 410.4 (found).

### N-[1-(dimethylamino)propan-2-yl]-2-(thiophen-2-yl)quinoline-4-carboxamide (6bg)

Yield: 73.3%. ¹H NMR (400 MHz, DMSO-d₆) δ 8.63 (d, J=8.3 Hz, 1H), 8.19 (dd, J=8.3, 1.3 Hz, 1H), 8.13-8.04 (m, 2H), 8.01 (dd, J=8.5, 1.2 Hz, 1H), 7.83-7.70 (m, 2H), 7.60 (ddd, J=8.2, 6.9, 1.3 Hz, 1H), 7.24 (dd, J=5.0, 3.7 Hz, 1H), 4.38-4.18 (m, 1H), 2.57-2.43 (m, 1H), 2.38-2.19 (m, 7H), 1.22 (d, J=6.6 Hz, 3H). ¹³C NMR (101 MHz, DMSO-d₆) δ 165.79, 151.57, 147.58, 144.28, 143.89, 130.30, 129.96, 128.72, 128.58, 127.58, 126.71, 125.60, 123.39, 115.24, 64.23, 45.36, 43.07, 18.79. C₁₉H₂₁N₃OS, EI-MS: m/z (M+H⁺): 340.5 (calculated), 340.7 (found).

### N-(1-methylazetidin-3-yl)-2-(thiophen-2-yl)quinoline-4-carboxamide (6bh)

Yield: 71.9%. ¹H NMR (400 MHz, DMSO-d₆) δ 9.52 (d, J=6.9 Hz, 1H), 8.23 (s, 1H), 8.17-8.07 (m, 2H), 8.07-7.94 (m, 1H), 7.83-7.71 (m, 2H), 7.59 (ddd, J=8.2, 6.9, 1.3 Hz, 1H), 7.24 (dd, J=5.0, 3.7 Hz, 1H), 4.73-4.53 (m, 1H), 3.87 (td, J=7.3, 1.8 Hz, 2H), 3.42 (td, J=6.9, 1.7 Hz, 2H), 2.46 (s, 3H). ¹³C NMR (101 MHz, DMSO-d₆) δ 166.00, 151.63, 147.62, 144.20, 142.39, 130.36, 130.08, 128.82, 128.60, 127.85, 126.87, 125.33, 123.23, 115.83, 61.88, 44.27. C₁₈H₁₇N₃OS, EI-MS: m/z (M+H⁺): 324.4 (calculated), 324.2 (found).

### N-(1-methylpyrrolidin-3-yl)-2-(thiophen-2-yl)quinoline-4-carboxamide (6bi)

Yield: 77.9%. ¹H NMR (400 MHz, DMSO-d₆) δ 9.10 (d, J=6.7 Hz, 1H), 8.18 (s, 1H), 8.12 (dd, J=3.7, 1.1 Hz, 1H), 8.07 (dd, J=8.4, 1.3 Hz, 1H), 8.00 (dd, J=8.6, 1.2 Hz, 1H), 7.83-7.71 (m, 2H), 7.59 (ddd, J=8.3, 6.9, 1.3 Hz, 1H), 7.24 (dd, J=5.0, 3.7 Hz, 1H), 4.66-4.46 (m, 1H), 3.11-2.98 (m, 1H), 2.95-2.74 (m, 2H), 2.74-2.60 (m, 1H), 2.45 (s, 3H), 2.37-2.24 (m, 1H), 1.98-1.81 (m, 1H). ¹³C NMR (101 MHz, DMSO-d₆) δ 166.20, 151.61, 147.56, 144.26, 142.92, 130.29, 130.01, 128.79, 128.57, 127.80, 126.79, 125.31, 123.33, 115.74, 61.19, 54.40, 49.14, 41.29, 31.09. C₁₉H₁₉N₃OS, EI-MS: m/z (M+H⁺): 338.4 (calculated), 338.2 (found).

### N-[2-(methylamino)ethyl]-2-(thiophen-2-yl)quinoline-4-carboxamide (6bj)

Yield: 71.3%. ¹H NMR (400 MHz, DMSO-d₆) δ 9.12 (t, J=5.6 Hz, 1H), 8.85 (s, 2H), 8.25 (s, 1H), 8.23-8.14 (m, 1H), 8.08-7.96 (m, 2H), 7.84-7.71 (m, 2H), 7.60 (ddd, J=8.2, 6.8, 1.3 Hz, 1H), 7.25 (dd, J=5.0, 3.7 Hz, 1H), 3.68 (q, J=6.0 Hz, 2H), 3.31-3.19 (m, 2H), 2.76-2.60 (m, 3H). ¹³C NMR (101 MHz, DMSO-d₆) δ 167.08, 151.60, 147.73, 144.20, 142.21, 130.41, 130.16, 128.83, 128.60, 127.63, 126.83, 125.67, 123.24, 116.06, 47.58, 35.78, 32.68. C₁₇H₁₇N₃OS, EI-MS: m/z (M+H⁺): 312.4 (calculated), 312.4 (found).

### N-{3-azabicyclo[3.1.0]hexan-6-yl}b-2-(thiophen-2-yl)quinoline-4-carboxamide (6bk)

Yield: 84.3%. ¹H NMR (400 MHz, DMSO-d₆) δ 9.09 (d, J=4.3 Hz, 1H), 8.15 (s, 1H), 8.12-8.03 (m, 2H), 8.03-7.94 (m, 1H), 7.84-7.69 (m, 2H), 7.60 (ddd, J=8.3, 6.9, 1.3 Hz, 1H), 7.24 (dd, J=5.0, 3.7 Hz, 1H), 3.51-3.37 (m, 5H), 3.15-3.10 (m, 1H), 2.14-2.05 (m, 2H). ¹³C NMR (101 MHz, DMSO-d₆) δ 167.22, 151.66, 147.62, 144.22, 142.61, 130.45, 130.14, 128.86, 128.64, 127.79, 126.94, 125.30, 123.19, 115.71, 46.64, 30.39, 22.92. C₁₉H₁₇N₃OS, EI-MS: m/z (M+H⁺): 336.4 (calculated), 336.2 (found).

### N-(2-aminoethyl)-2-(thiophen-2-yl)quinoline-4-carboxamide (6b1)

Yield: 76.3%. ¹H NMR (400 MHz, DMSO-d₆) δ 9.11 (t, J=5.6 Hz, 1H), 8.27 (s, 1H), 8.25-8.09 (m, 4H), 8.09-7.97 (m, 2H), 7.78 (dd, J=8.3, 6.1 Hz, 2H), 7.60 (ddd, J=8.3, 6.9, 1.3 Hz, 1H), 7.25 (dd, J=5.1, 3.7 Hz, 1H), 3.64 (q, J=6.1 Hz, 2H), 3.12 (t, J=6.3 Hz, 2H). ¹³C NMR (101 MHz, DMSO-d₆) δ 167.06, 151.64, 147.72, 144.24, 142.34, 130.39, 130.14, 128.82, 128.59, 127.66, 126.83, 125.65, 123.27, 116.07, 38.37, 37.18. C₁₆H₁₅N₃OS, EI-MS: m/z (M+H⁺): 298.4 (calculated), 298.4 (found).

### N-(1-aminopropan-2-yl)-2-(thiophen-2-yl)quinoline-4-carboxamide (6bm)

Yield: 88.3%. ¹H NMR (400 MHz, DMSO-d₆) δ 8.96 (d, J=7.9 Hz, 1H), 8.39-8.20 (m, 4H), 8.16 (dd, J=8.4, 1.3 Hz, 1H), 8.08 (dd, J=3.8, 1.2 Hz, 1H), 8.01 (dd, J=8.5, 1.2 Hz, 1H), 7.86-7.71 (m, 2H), 7.60 (ddd, J=8.3, 6.9, 1.3 Hz, 1H), 7.25 (dd, J=5.0, 3.7 Hz, 1H), 4.48-4.28 (m, 1H), 3.11-2.94 (m, 2H), 1.29 (d, J=6.7 Hz, 3H). ¹³C NMR (101 MHz, DMSO-d₆) δ 166.55, 151.65, 147.69, 144.28, 142.52, 130.35, 130.12, 128.83, 128.57, 127.74, 126.82, 125.59, 123.32, 116.09, 43.78, 43.11, 18.11. C₁₇H₁₇N₃OS, EI-MS: m/z (M+H⁺): 312.4 (calculated), 312.3 (found).

### N-[(azetidin-3-yl)methyl]-2-(thiophen-2-yl)quinoline-4-carboxamide (6bn)

Yield: 69.3%. ¹H NMR (400 MHz, DMSO-d₆) δ 9.41-8.94 (m, 3H), 8.20 (s, 1H), 8.16-8.05 (m, 2H), 8.01 (dd, J=8.5, 1.1 Hz, 1H), 7.85-7.71 (m, 2H), 7.60 (ddd, J=8.2, 6.8, 1.2 Hz, 1H), 7.24 (dd, J=5.0, 3.7 Hz, 1H), 4.16-3.97 (m, 2H), 3.97-3.81 (m, 2H), 3.61 (t, J=6.1 Hz, 2H), 3.25-3.00 (m, 1H). ¹³C NMR (101 MHz, DMSO-d₆) δ 167.15, 151.69, 147.66, 144.23, 142.81, 130.41, 130.13, 128.86, 128.61, 127.88, 126.91, 125.42, 123.26, 115.76, 48.51, 40.86, 31.74. C₁₈H₁₇N₃OS, EI-MS: m/z (M+H⁺): 324.4 (calculated), 324.7 (found).

### N-[2-(dimethylamino)ethyl]-2-(furan-2-yl)quinoline-4-carboxamide (6bo)

Yield: 91.3%. ¹H NMR (400 MHz, DMSO-d₆) δ 8.82 (t, J=5.7 Hz, 1H), 8.15 (ddd, J=8.4, 1.4, 0.6 Hz, 1H), 8.04 (ddd, J=8.4, 1.3, 0.6 Hz, 1H), 8.00-7.90 (m, 2H), 7.79 (ddd, J=8.4, 6.9, 1.5 Hz, 1H), 7.60 (ddd, J=8.2, 6.9, 1.3 Hz, 1H), 7.43 (dd, J=3.4, 0.8 Hz, 1H), 6.74 (dd, J=3.4, 1.8 Hz, 1H), 3.49 (q, J=6.5 Hz, 2H), 2.56 (t, J=6.7 Hz, 2H), 2.29 (s, 6H). ¹³C NMR (101 MHz, DMSO-d₆) δ 166.42, 152.67, 147.96, 147.79, 145.24, 143.36, 130.35, 129.00, 126.88, 125.53, 123.22, 115.06, 112.68, 111.18, 57.81, 48.59, 44.99, 37.13. C₁₈H₁₉N₃O₂, EI-MS: m/z (M+H⁺): 310.4 (calculated), 310.4 (found).

### N-(2-aminoethyl)-2-(furan-2-yl)quinoline-4-carboxamide (6 bp)

Yield: 77.9%. ¹H NMR (400 MHz, DMSO-d₆) δ 9.07 (t, J=5.6 Hz, 1H), 8.26-8.08 (m, 5H), 8.08-8.00 (m, 1H), 7.97 (dd, J=1.8, 0.8 Hz, 1H), 7.80 (ddd, J=8.4, 6.9, 1.5 Hz, 1H), 7.61 (ddd, J=8.2, 6.9, 1.3 Hz, 1H), 7.42 (dd, J=3.5, 0.8 Hz, 1H), 6.76 (dd, J=3.4, 1.8 Hz, 1H), 3.63 (q, J=6.1 Hz, 2H), 3.16-3.01 (m, 2H). ¹³C NMR (101 MHz, DMSO-d₆) δ 167.03, 152.70, 148.01, 147.87, 145.30, 142.38, 130.40, 129.04, 126.94, 125.65, 123.12, 115.68, 112.75, 111.25, 38.40, 37.18. C₁₆H₁₅N₃O₂, EI-MS: m/z (M+H⁺): 282.3 (calculated), 282.4 (found).

### N-(azetidin-3-yl) 2-(furan-2-yl)quinoline-4-carboxamide (6bq)

Yield: 75.3%. ¹H NMR (400 MHz, DMSO-d₆) δ 9.65 (d, J=6.6 Hz, 1H), 9.19 (s, 2H), 8.16 (ddd, J=8.4, 1.5, 0.6 Hz, 1H), 8.12-8.03 (m, 2H), 7.98 (dd, J=1.8, 0.8 Hz, 1H), 7.81 (ddd, J=8.4, 6.9, 1.5 Hz, 1H), 7.62 (ddd, J=8.3, 6.9, 1.3 Hz, 1H), 7.43 (dd, J=3.4, 0.8 Hz, 1H), 6.76 (dd, J=3.4, 1.8 Hz, 1H), 5.04-4.81 (m, 1H), 4.35-4.23 (m, 2H), 4.23-4.00 (m, 2H). ¹³C NMR (101 MHz, DMSO-d₆) δ 166.26, 152.58, 147.99, 147.87, 145.38, 141.90, 130.55, 129.12, 127.13, 125.40, 122.95, 115.54, 112.78, 111.32, 51.85, 41.61. C₁₇H₁₅N₃O₂, EI-MS: m/z (M+H⁺): 294.3 (calculated), 294.3 (found).

### N-[(azetidin-3-yl)methyl]-2-(furan-2-yl)quinoline-4-carboxamide (6br)

Yield: 84.3%. ¹H NMR (400 MHz, DMSO-d₆) δ 9.23-8.85 (m, 3H), 8.10 (ddd, J=8.5, 1.5, 0.6 Hz, 1H), 8.08-7.98 (m, 2H), 7.97 (dd, J=1.7, 0.8 Hz, 1H), 7.80 (ddd, J=8.4, 6.9, 1.5 Hz, 1H), 7.61 (ddd, J=8.3, 6.9, 1.3 Hz, 1H), 7.45 (dd, J=3.4, 0.8 Hz, 1H), 6.75 (dd, J=3.4, 1.8 Hz, 1H), 4.18-3.96 (m, 2H), 3.96-3.76 (m, 2H), 3.61 (t, J=6.2 Hz, 2H), 3.17-3.01 (m, 1H). ¹³C NMR (101 MHz, DMSO-d₆) δ 167.06, 152.63, 148.00, 147.83, 145.33, 142.75, 130.43, 129.09, 127.04, 125.44, 123.10, 115.37, 112.72, 111.39, 48.58, 40.91, 31.69. C₁₈H₁₇N₃O₂, EI-MS: m/z (M+H⁺): 308.4 (calculated), 308.6 (found).

### N-(2-aminoethyl)-8-chloro-2-(furan-2-yl)quinoline-4-carboxamide (6bs)

Yield: 85.6%. ¹H NMR (400 MHz, DMSO-d₆) δ 9.16 (t, J=5.5 Hz, 1H), 8.32-8.07 (m, 5H), 8.04-7.93 (m, 2H), 7.57 (dd, J=8.4, 7.5 Hz, 1H), 7.48 (dd, J=3.5, 0.8 Hz, 1H), 6.78 (dd, J=3.5, 1.7 Hz, 1H), 3.71-3.57 (m, 2H), 3.15-2.99 (m, 2H). ¹³C NMR (101 MHz, DMSO-d₆) δ 166.38, 152.15, 148.19, 145.57, 143.54, 142.77, 132.11, 130.24, 126.71, 124.78, 124.35, 116.41, 112.62, 112.05, 38.07, 36.97. C₁₆H₁₄ClN₃O₂, EI-MS: m/z (M+H⁺): 316.8 (calculated), 316.8 (found).

### Biological Evaluation

### Cell Lines, Viruses, and Viral Infection.

Cell culture experiments were performed using the following cell lines: rhabdomyosarcoma (RD) (CCL-136; ATCC) and Vero C1008 (ATCC). Cells were maintained in DMEM supplemented with 10% fetal bovine serum (FBS; Gibco) and 1% penicillin-streptomycin. All cells were placed in a 37° C. incubator under a 5% CO2 atmosphere. The following reagents were obtained through BEI resources, NIAID, NIH: EVD68 strains US/MO/14-18947 (NR-49129) and US/KY/14-18953 (NR-49132), and EVA71 strain Tainan/4643/1998 (NR-471). EV-A71 strain USA/AK/2016-19516 was kindly provided by Dr. William Allan Nix at the Centers for Disease Control and Prevention. CVB3 strain Nancy (ATCC® VR-30) was ordered from ATCC. All viruses were propagated in RD cells except for CVB3 Nancy which was propagated in Vero cells.

### Cytotoxicity Assay (CCso) and Antiviral Cytopathic Effect Assay (CPE)

Assays used for determining compound cytotoxicity (CC₅₀ values) and antiviral efficacy (EC₅₀ values) were performed on 96-well cell culture plates as previously described (see, R. Musharrafieh, et al., J. Med. Chem. 62 (2019) 4074-4090). Briefly, cells were seeded to approximately 90% confluency the next day. For antiviral activity assays, growth media was removed and cells were washed with phosphate-buffered saline (PBS) supplemented with magnesium and calcium, and infected with approximately 0.01 MOI of virus or until complete CPE was observed after 3 days of infection. All strains were diluted in DMEM with 2% FBS, but EV-D68 diluted media was further supplemented with 30 mM MgCl2 during and after infection. EV-A71 and CVB3 infected cells were incubated at 37° C., and EV-D68 infected cells were placed in a 33° C. incubator, both at 5% CO2. For staining, viral media was removed and cells were treated with a pre-warmed DMEM solution of 66 μg/mL neutral red for 1-2h at 37° C. Neutral red was then removed and cells were washed and allowed to dry at room temperature followed by de-staining. Neutral red uptake was measured using a Multiskan FC microplate photometer (Thermo Fisher Scientific) at an absorption reading of 540 nm. For cytotoxicity assay, CC₅₀ values were obtained after 3 days of compound addition using the same staining protocol as described above but in the absence of viral infection.

### Mouse Microsomal Stability

Test compounds were incubated at 37° C. with liver microsomes (mouse or human) (Xenotech) at 1 μM drug concentration in the presence of a NADPH regenerating system at 0.5 mg/ml microsomal protein. Testosterone (3A4 substrate), Propafenone (2D6), and Diclofenac (2C9) were included as positive controls. Compounds were incubated with microsomes in the presence of a NADPH regenerating system (Sigma, Cat. No. N0505, Lot SLBH3107V). Aliquots at 0, 5, 10, 20, 30 and 60 minutes post incubation were collected and immediately mixed with cold acetonitrile containing internal standard (IS). Compounds incubated with microsomes without NADPH regenerating system for 60 minutes were also included. All the samples were analyzed by LC/MS/MS; disappearance of test compound were assessed based on peak area ratios of analyte/IS. Following equations were applied to calculate the microsome clearance:

$$Elimination rate constant ( k ) = - gradient ; Half life ( {T}_{1 / 2} ) ( min ) = \frac{0.693}{k}$$

CL_{int(mic)}=0.693/half life/mg microsome protein per mL; Liver wt: 40 g/kg, 30 g/kg, 32 g/kg, 20 g/kg and 88 g/kg for rat, monkey, dog, human and mouse; Using CL_{int(mic)} to calculate the whole the liver clearance: mg microsomal protein/g liver weight: 45 mg/g for 5 species; CLᵢₙₜ₍ₗᵢᵥₑᵣ₎=CL_{int(mic)}*mg microsomal protein/g liver weight*g liver weight/kg body weight.

### CVB3 2C Protein Expression

The DNA fragment coding for CVB3 Nancy 2C (amino acids 37 to 329) protein with E. coli codon optimization was ordered from GenScript (Piscataway, N.J.) and inserted into the pET28a (+)-TEV vector. The recombinant plasmid was transformed into E. coli Rosetta 2(DE3) competent cells and bacterial cultures were grown in LB containing 50 μg/mL of kanamycin and 34 μg/mL of chloramphenicol. Cell cultures were grown at 37° C. with shaking, and expression of the target protein was induced by the addition of 0.5 mM of isopropyl β-d-1-thiogalactopyranoside (IPTG) when the optical density (A600) reached 0.6-0.8. Growth of bacterial cultures were continued at 18° C. for additional 12-16 h postinduction, and the bacteria were harvested by centrifugation (6000 g, 10 min, 4° C.) and resuspended in lysis buffer (20 mM Hepes [pH 7.5], 300 mM NaCl, 1 mg/ml lysozyme, 1 mM phenylmethylsulfonyl fluoride [PMSF], 0.01 mg/ml DNase I), and lysed by sonication for 30 mins. The cell debris were removed by centrifugation at 17,000 g for 1 hr. The supernatant was incubated with nickel-nitrilotriacetic acid (Ni-NTA) resin for over 2 h at 4° C. and CVB3 2C (37-329) was purified to greater than 98% homogeneity by eluting with increasing concentrations of imidazole. Finally, the protein was dialyzed twice against a buffer containing 20 mM Hepes (pH 7.5), 300 mM NaCl and 1 mM DTT. Purified proteins were fast frozen in liquid nitrogen and stored at −80° C. freezer.

### Thermal Shift Assay

The binding of compounds on CVB3 2C protein was monitored by differential scanning fluorimetry (DSF) using a Thermal Fisher QuantStudio™ 5 Real-Time PCR System. TSA plates were prepared by mixing CVB3 2C protein (final concentration of 4.5 μM in 20 mM Hepes pH 7.5, 300 mM NaCl, 1 mM DTT) with either compound 6aw (10, 30 or 100 μM in the assay with 2% DMSO), or fluoxetine (100 μM), or guanidine chloride (1 mM), and 1×SYPRO orange (Thermal Fisher) in a final volume of 50 μl and incubated at 30° C. for 1 hr. The experiments were performed under a temperature gradient ranging from 20 to 95° C. (incremental steps of 0.05° C./s). The melting temperature (Tₘ) was calculated as the mid-log of the transition phase from the native to the denatured protein using a Boltzmann model (Protein Thermal Shift Software v1.3). Thermal shift ΔTₘ was calculated by subtracting reference melting temperature of protein alone from the Tₘ in the presence of compound.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound described by Formula I: 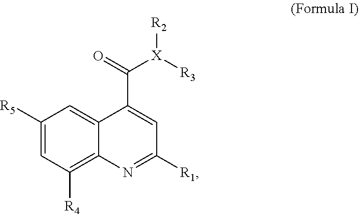
including pharmaceutically acceptable salts, solvates, and/or prodrugs thereof; wherein X, R1, R2, R3, R4 and R5 independently include any chemical moiety that permits the resulting compound to inhibit enterovirus activity and/or expression.

2. The compound of claim 1, wherein the enterovirus is a non-polio enterovirus.

3. The compound of claim 1, wherein the enterovirus is EV-D68, EV-A71, or CVB3.

4. The compound of claim 1, wherein Xis oxygen or nitrogen such that if X is oxygen than either R2 or R3 is absent.

5. The compound of claim 1, wherein R1 is selected from hydrogen,

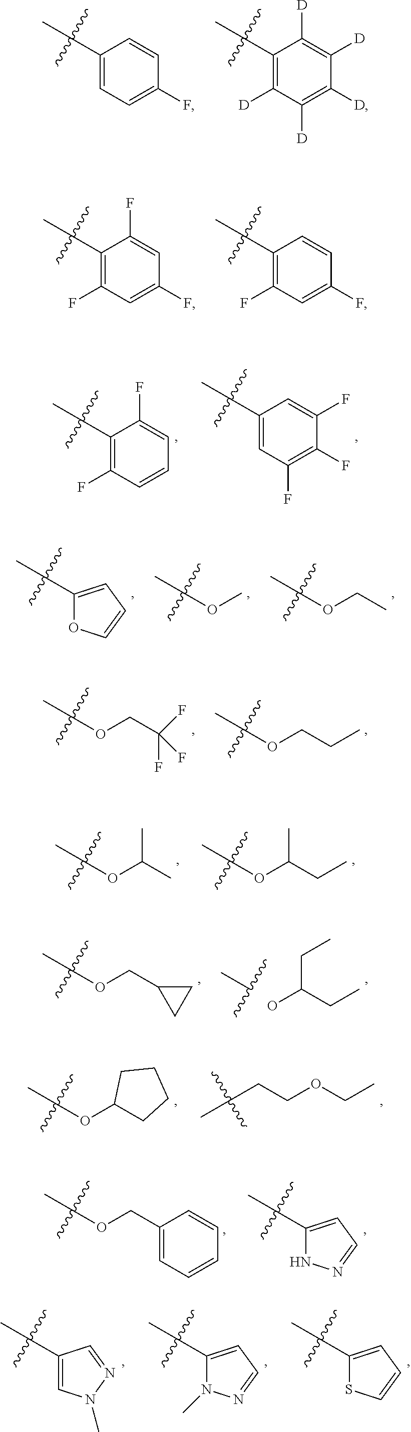

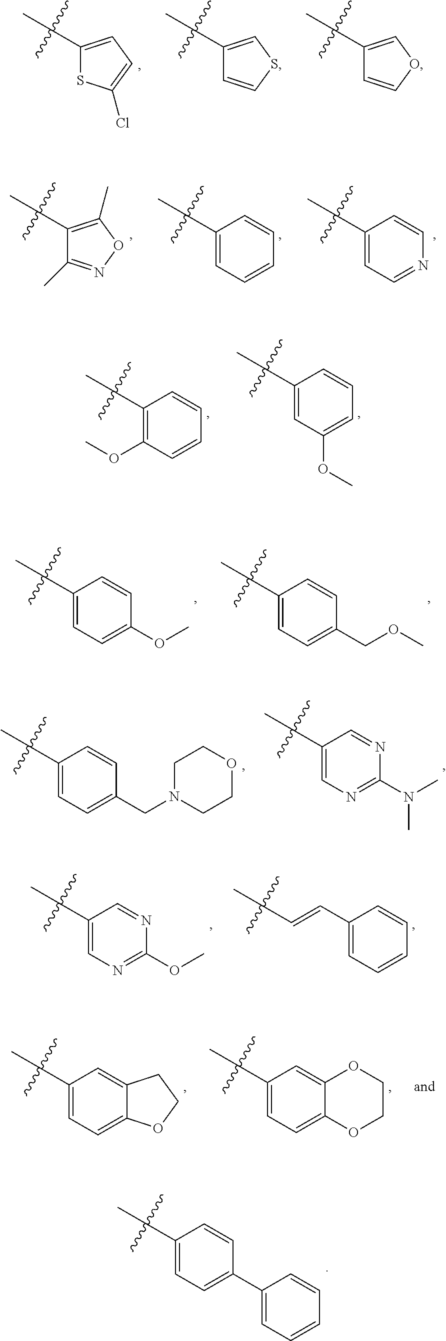

6. The compound of claim 1,
wherein R2 and R3 are independently selected from hydrogen, 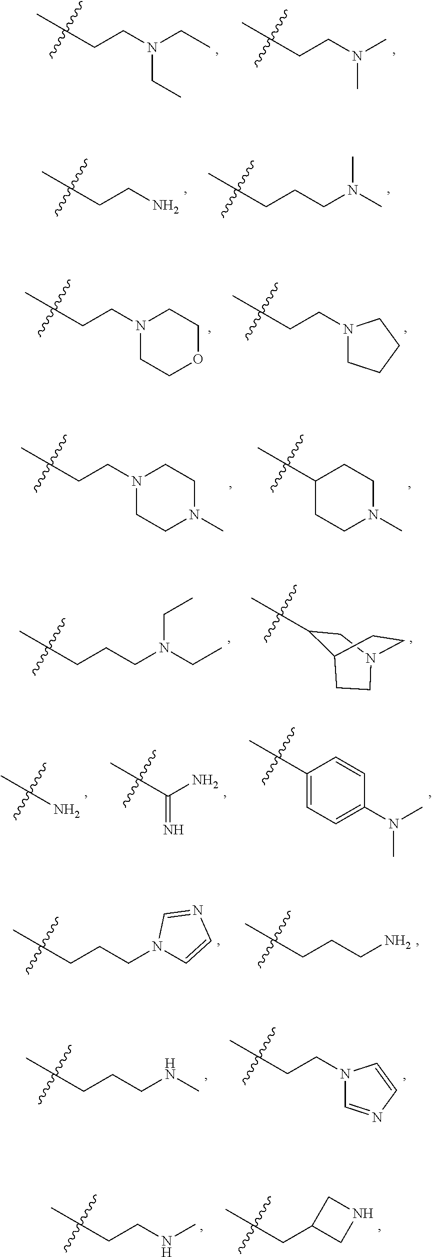 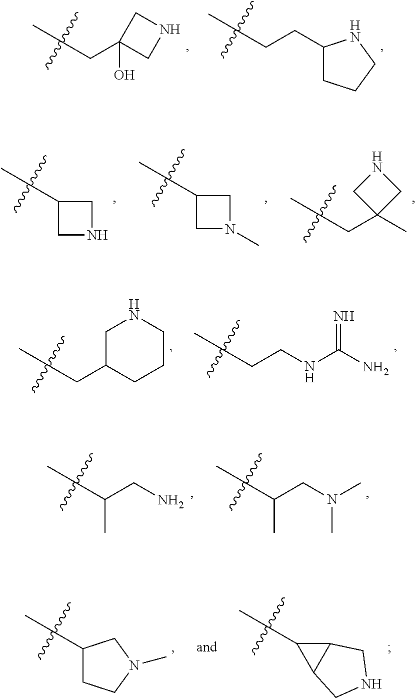
or
wherein R2 and R3 combine such that

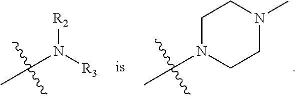

7. The compound of claim 1, wherein R4 is selected from hydrogen and halogen (e.g., chlorine).

8. The compound of claim 1, wherein R5 is selected from hydrogen and halogen (e.g., chlorine, fluorine).

9. The compound of claim 1, wherein the compound is selected from any of the compounds recited in Tables 2 (excluding dibucaine), 3, 4, 5, 6 and 9.

10. The compound of claim 1, wherein the compound is comprised within a pharmaceutical composition.

11. A kit comprising a compound of claim 1 and instructions for administering said compound to a patient having a disease or condition characterized with enterovirus activity and/or expression.

12. A method of treating, ameliorating, or preventing a disease or condition characterized with enterovirus activity and/or expression, comprising administering to a patient a therapeutically effective amount of the pharmaceutical composition of claim 10.

13. The method of claim 12, wherein the enterovirus is a non-polio enterovirus.

14. The method of claim 12, wherein the enterovirus is EV-D68, EV-A71, or CVB3.

15. The method of claim 12, wherein the disease or condition is a respiratory illness or a neuronal condition (e.g., acute flaccid myelitis (AFM)).

16. The method of claim 12, wherein the patient is a human patient.

17. The method of claim 12, further comprising administering to said patient one or more antiviral agents.
